(19) United States
(12) Patent Application Publication
(10) Pub. No.: US 2011/0178110 A1
(43) Pub. Date: Jul. 21, 2011

(54) Genotype and Expression Analysis for Use in Predicting Outcome and Therapy Selection

(75) Inventors: Heinz-Josef Lenz, Los Angeles, CA (US); Wu Zhang, Los Angeles, CA (US)

(73) Assignee: University of Southern California

(21) Appl. No.: 12/992,584

(22) PCT Filed: May 14, 2009
(86) PCT No.: PCT/US09/44043

§ 371 Date: Feb. 10, 2011

Related U.S. Application Data
(60) Provisional application No. 61/053,634, filed on May 15, 2008.
(60) Provisional application No. 61/057,758, filed on May 30, 2008.

(51) Int. Cl. A61K 31/513; C12Q 1/68; A61P 35/00

(52) U.S. Cl. 514/274; 435/6.11; 435/6.12; 435/6.1

(57) ABSTRACT

The invention provides compositions and methods for determining the likelihood of successful treatment with a various treatment regimens available to gastrointestinal cancer patients. After determining if a patient is likely to be successfully treated, the invention also provides methods for treating these patients.

### CROSS REFERENCE TO RELATED APPLICATIONS

This application claims the benefit under 35 U.S.C. §119(e) of U.S. Provisional Application Nos. 61/053,634, filed on May 15, 2008 and 61/057,758, filed on May 30, 2008, the contents of which are hereby incorporated by reference into the present disclosure.

### FIELD OF THE INVENTION

This invention relates to the field of pharmacogenomics and specifically to the application of genetic polymorphisms or gene expression levels to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) J. Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniques 39:565-568.

The expression level of a variety of genes also have been linked to cancer prognosis and treatment protocol selection. Microarray gene expression profiling has been used to predict the prognosis of patients suffering from cancers including colon cancer, breast cancer, lung cancer and lymphomas. Barrier et al. (2005) Oncogene 24:6155-6164. Tumor tissue in these cancers have been shown to have both overexpression and underexpression of prognostic predictive genes. Furthermore, not only the tumor tissue itself can be predictive, but tumor-adjacent normal tissue has been show to predict tumor recurrence in patients suffering from rectal cancer treated with adjuvant chemoradiation. Schneider et al. (2006) Pharmacogenetics and Genomics 16(8):555-563. One recent study (Yang et al. (2006) Clinical Colorectal Cancer 6(4):305-311) showed that gene expression levels of EGFR, Survivin and VEGF in tumor tissue were predictive markers for lymph node involvement in patients with locally advanced rectal cancer treated with surgical resection and adjuvant chemoradiation therapy.

Although considerable research correlating gene expression and/or polymorphisms has been reported, much work remains to be done. This invention supplements the existing body of knowledge and provides related advantages as well.

### DESCRIPTION OF THE EMBODIMENTS

This invention provides methods for identifying a gastrointestinal cancer patient that is more likely to experience tumor recurrence following surgical resection of a tumor, comprising, or alternatively consisting essentially of, or yet further consisting of screening a suitable patient tissue or cell sample for one genotype of the group PAR-1 I-506D, ES G+4349A or IL-8 T-251A polymorphisms, wherein (ins/ins) for Par-1 I-506D; (A/A) for IL-8 T-251A; or (A/A) for ES G+4349A, respectively, identifies the patient as more likely to experience tumor recurrence following surgical resection of a tumor.

Also provided herein are methods for identifying a gastrointestinal cancer patient that is less likely to experience tumor recurrence following surgical resection of a tumor, comprising or alternatively consisting essentially of, or yet further consisting of, screening a suitable patient tissue or cell sample for sample for one genotype of the group PAR-1 I-506D, ES G+4349A or IL-8 T-251A polymorphisms, wherein (del/del or ins/del) for Par-1 I-506D; (T/T or T/A) for IL-8 T-251A; or (G/G or G/A) for ES G+4349A, respectively, identifies the patient as less likely to experience tumor recurrence following surgical resection of a tumor

This invention also provides methods for identifying a stage II colon cancer patient that is more likely to show responsiveness to 5-FU based adjuvant chemotherapy regimen or equivalent thereof, comprising, or alternatively consisting essentially of, or yet further consisting of, screening a suitable cell or tissue sample for at least one genotype of IL-1β C+3954T, IL-1Ra VNTR or VEGF G-634C polymorphisms, wherein (C/C or C/T) for IL-1β C+3954T; (4 repeats/4 repeats or 2 repeats/2 repeats) for IL-1Ra VNTR or (G/G) for VEGF G-634C, respectively, identifies the patient as more likely to show responsive to said therapy.

Also provided are methods for identifying a stage II colon cancer patient that is more likely to experience tumor recurrence following 5-FU based adjuvant chemotherapy regimen or equivalent thereof, comprising, or alternatively consisting essentially of, or yet further consisting of, screening a suitable patient cell or tissue sample for at least one genotype of IL-1β C+3954T, IL-1Ra VNTR or VEGF G-634C, wherein (T/T) for IL-1β C+3954T; (at least one allele with >4 repeats) for IL-1Ra VNTR; or (C/C or C/G) for VEGF G-634C, respectively, identifies the patient as more likely to experience tumor recurrence following said therapy.

Yet further provided are methods for selecting a therapy comprising 5-FU based adjuvant chemotherapy regimen or equivalent thereof for a stage II colon cancer patient in need thereof, comprising, or alternatively consisting essentially of, or yet further consisting of, screening a suitable patient cell or tissue sample for the presence of a genotype (C/C or C/T) for IL-1β C+3954T; (4 repeats/4 repeats or 2 repeats/2 repeats) for IL-1Ra VNTR or (G/G) for VEGF G-634C, respectively, wherein the presence of said genotype selects said patient for said chemotherapy.

Also provided are methods for treating a stage II colon cancer patient selected for therapy comprising, or alternatively consisting essentially of, or yet further consisting of, administration of a 5-FU based adjuvant chemotherapy regimen or equivalent thereof, comprising, or alternatively consisting essentially of, or yet further consisting of screening a suitable cell or tissue sample for the presence of a genotype (C/C or C/T) for IL-1β C+3954T; (4 repeats/4 repeats or 2 repeats/2 repeats) for IL-1Ra VNTR; or (G/G) for VEGF G-634C, and administering an effective amount of said chemotherapy to a patient having a genotype identified above, thereby treating said patient.

Methods for identifying a gastrointestinal cancer patient that is more likely to show responsiveness to first line FOLFOX/BV or first line XELOX/BV chemotherapy regimen or equivalent of each thereof is provided by screening a suitable patient cell or tissue sample for at least one genotype of the group of ICAM-1 codon K496E, GRP78 (rs12009), or NFkB CA repeat, wherein (C/C or C/T) for ICAM-1 codon K496E; (C/C or C/T) for GRP78 (rs12009); or (at least 1 allele with ≧24 CA repeats) for NFkB CA repeat, respectively, identifies the patient as more likely to show responsiveness to said therapy.

Also provided are methods for identifying a gastrointestinal cancer patient that is less likely to show responsiveness to first line FOLFOX/BV or first line XELOX/BV chemotherapy regimen or equivalent of each thereof, comprising, or alternatively consisting essentially of, or yet further, consisting of screening a suitable patient cell or tissue sample for at least one genotype of the group of ICAM-1 codon K496E, GRP78 (rs12009), or NFkB CA repeat, wherein (T/T) for ICAM-1 codon K496E; (T/T) for GRP78 (rs12009); or (two alleles with <24 CA repeats) for NFkB CA repeat, respectively, identifies the patient as less likely to show responsiveness to said therapy.

Further provided are methods for selecting a therapy comprising first line FOLFOX/BV or first line XELOX/BV chemotherapy regimen or equivalent of each thereof for a gastrointestinal patient in need thereof, comprising, or alternatively consisting essentially of, or yet further consisting of screening a suitable cell or tissue sample for at least one genotype of the group (C/C or C/T) for ICAM-1 codon K496E; (C/C or C/T) for GRP78 (rs12009); or (at least 1 allele with ≧24 CA repeats) for NFkB CA repeat, wherein the presence of at least one of said genotype selects the patient for said chemotherapy regimen.

Yet further provided are methods for treating a gastrointestinal cancer patient selected for therapy comprising, or alternatively consisting essentially of, or yet further consisting of, administration of a first line FOLFOX/BV or first line XELOX/BV chemotherapy regimen or equivalent of each thereof, comprising, or alternatively consisting essentially of, or yet further consisting of, screening a suitable patient cell or tissue sample for the presence of at least one genotype of the group: (C/C or C/T) for ICAM-1 codon K496E; (C/C or C/T) for GRP78 (rs12009); or (at least 1 allele with ≧24 CA repeats) for NFkB CA repeat, administering an effective amount of said chemotherapy to a patient having at least one genotype identified above, thereby treating said patient. Methods of determining an effective amount are known in the art and can be empirically determined by the treating physician.

This invention also provides methods for identifying a gastrointestinal cancer patient that is more likely to show responsiveness to FOLFOX/BV or XELOX/BV chemotherapy regimen or equivalent thereof, comprising, or alternatively consisting essentially of, or yet further consisting of, screening a suitable patient cell or tissue sample for at least one genotype of K-RAS codon 12 or K-RAS codon 13, wherein a wild type K-RAS codon 12 (GGT) and a wild type K-RAS codon 13 (GGC), respectively, of the K-RAS gene identifies the patient as more likely to show responsive to said therapy.

Further provided are methods for identifying a gastrointestinal cancer patient that is less likely to show responsiveness to FOLFOX/BV or XELOX/BV chemotherapy regimen or equivalent thereof, comprising, or alternatively consisting essentially of, or yet further consisting of, screening a suitable patient cell or tissue sample for at least one genotype of K-RAS codon 12 or K-RAS codon 13, wherein a mutation in K-RAS codon 12 or K-RAS codon 13 of the K-RAS gene, respectively, identifies the patient as less likely to show responsive to said therapy.

Also provided are methods for selecting a therapy comprising FOLFOX/BV or XELOX/BV chemotherapy regimen or equivalent thereof for a gastrointestinal cancer patient in need thereof, comprising, or alternatively consisting essentially of, or yet further consisting of, screening a suitable patient cell or tissue sample for the presence of a wild type K-RAS codon 12 (GGT) and a wild type K-RAS codon 13 (GGC) genotype of the K-RAS gene selects said patient for said chemotherapy.

Yet further are methods for treating a gastrointestinal cancer patient selected for therapy comprising, or alternatively consisting essentially of, or yet further consisting of, administration of a FOLFOX/BV or XELOX/BV chemotherapy regimen or equivalent thereof, comprising, or alternatively consisting essentially of, or yet further consisting of, screening a suitable patient cell or tissue sample for the presence of a wild type K-RAS codon 12 (GGT) and a wild type K-RAS codon 13 (GGC) genotype of the K-RAS gene; and administering an effective amount of said chemotherapy to a patient having a genotype identified in step a, thereby treating said patient. Methods of determining an effective amount are known in the art and can be empirically determined by the treating physician.

This invention also provides methods for identifying a stage II or stage III rectal cancer patient that is more likely to experience longer relative overall survival or progression fee survival following treatment comprising, or alternatively consisting essentially of, or yet further consisting of, the administration of 5-FU or an equivalent thereof and pelvic radiation, comprising, or alternatively consisting essentially of, or yet further consisting of, screening a suitable patient tissue or cell sample for the expression level of the thymidylate synthase gene, wherein low expression of the gene identifies the patient as more likely to experience longer relative overall survival or progression fee survival following said therapy.

Further provided are methods for identifying a stage II or stage III rectal cancer patient that is more likely to experience shorter relative overall survival or progression fee survival following treatment comprising, or alternatively consisting essentially of, or yet further consisting of, the administration of 5-FU or an equivalent thereof and pelvic radiation, comprising, or alternatively consisting essentially of, or yet further consisting of, screening a suitable patient tissue or cell sample for the expression level of the thymidylate synthase gene, wherein high or medium expression of the gene identifies the patient as more likely to experience shorter relative overall survival or progression fee survival following said therapy.

Yet further provided is a method for selecting therapy comprising, or alternatively consisting essentially of, or yet further consisting of, the administration of 5-FU or an equivalent thereof and pelvic radiation to a stage II or stage III rectal cancer patient in need thereof, comprising, or alternatively consisting essentially of, or yet further consisting of, determining the expression level of the thymidylate synthase gene in a suitable patient tissue or cell sample, wherein low expression of said gene selects the patient for said therapy.

Also provided are methods for treating a stage II or stage III rectal cancer patient selected for treatment comprising administration of an effective amount of 5-FU or an equivalent thereof and pelvic radiation, the method comprising, or alternatively consisting essentially of, or yet further consisting of, determining the expression level of the thymidylate synthase gene in a suitable patient tissue or cell sample, administering an effective amount of said treatment to a patient having low expression of said gene, thereby treating the patient.

Also provided are methods for identifying a gastrointestinal cancer patient that is more likely responsive to therapy comprising, or alternatively consisting essentially of, or yet further consisting of, first line FOLFOX in combination with PTK/ZK chemotherapy or equivalent of each thereof, comprising, or alternatively consisting essentially of, or yet further consisting of, determining the expression level of at least one gene of the group LDHA, Glut1, or VEGFR1 in a suitable tissue or cell sample, wherein high LDHA expression, high Glut1 expression, or high VEGFR1 expression, respectively, identifies the patient that is more likely responsive to said therapy.

Further provided are methods for identifying a gastrointestinal cancer patient that is more likely responsive to therapy comprising, or alternatively consisting essentially of, or yet further consisting of, second line FOLFOX in combination with PTK/ZK chemotherapy or equivalent of each thereof, comprising, or alternatively consisting essentially of, or yet further consisting of, determining the expression level of HIF1α in a suitable patient tissue or cell sample, wherein low HIF1α expression identifies the patient that is more likely responsive to said therapy.

Yet further provided are methods for identifying a gastrointestinal cancer patient that is more likely to have progression free survival following therapy comprising, or alternatively consisting essentially of, or yet further consisting of, first line FOLFOX in combination with PTK/ZK chemotherapy or equivalent of each thereof, comprising, or alternatively consisting essentially of, or yet further consisting of, determining the expression level of at least one gene of the group VEGFR1 or LDHA in a suitable patient tissue or cell sample, wherein high VEGFR1 expression or high LDHA expression, respectively, identifies the patient that is more likely to have progression free survival following said therapy.

Also provided are methods for identifying a gastrointestinal cancer patient that is more likely to have progression free survival following therapy comprising, or alternatively consisting essentially of, or yet further consisting of, second line FOLFOX in combination with PTK/ZK chemotherapy or equivalent of each thereof, comprising, or alternatively consisting essentially of, or yet further consisting of, determining the expression level a HIF1α gene in a suitable tissue or cell sample, wherein low HIFα expression identifies the patient that is more likely to have progression free survival following said therapy.

Also provided are methods for identifying a gastrointestinal cancer patient that is more likely to have longer overall survival following therapy comprising, or alternatively consisting essentially of, or yet further consisting of, first line FOLFOX chemotherapy or an equivalent thereof, comprising, or alternatively consisting essentially of, or yet further consisting of, determining the expression level of at least one gene of the group HIF1α or VEGFR2 in a suitable patient tissue or cell sample, wherein low HIF1α expression or low VEGFR2 expression identifies the patient that is more likely to have longer overall survival following said therapy.

Alternatively, methods for identifying a gastrointestinal cancer patient that is more likely to have longer overall survival following therapy comprising second line FOLFOX in combination with PTK/ZK chemotherapy or equivalent of each thereof, comprising, or alternatively consisting essentially of, or yet further consisting of, determining the expression level of Glut1 in a suitable patient tissue or cell sample, wherein low Glut1 expression identifies the patient that is more likely to have longer overall survival following said therapy.

Also provided are method for selecting first line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely responsive to said therapy, comprising, or alternatively consisting essentially of, or yet further consisting of, determining the expression level of at least one gene of the group LDHA, Glut1 or VEGFR1 in a suitable patient tissue or cell sample, wherein high LDHA expression, high Glut1 expression, or high VEGFR1 expression, respectively, selects the patient for said therapy.

Also provided are methods for selecting second line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely responsive to said therapy, comprising, or alternatively consisting essentially of, or yet further consisting of, determining the expression level of HIF1α in a suitable patient tissue or cell sample, wherein low HIF1 expression selects the patient for said therapy.

Yet further are provided methods for selecting first line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely to experience longer progression free survival, comprising, or alternatively consisting essentially of, or yet further consisting of, determining the expression level of at least one gene of the group VEGFR1 or LDHA in a suitable patient tissue or cell sample, wherein high VEGFR1 expression or high LDHA expression, respectively, selects the patient for said therapy.

This invention also provides methods for selecting second line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely to experience longer progression free survival comprising, or alternatively consisting essentially of, or yet further consisting of, determining the expression level of a HIF1α gene in a suitable patient tissue or cell sample, wherein low HIF1α expression selects the patient for said therapy.

Also provided are methods for selecting first line therapy comprising, or alternatively consisting essentially of, or yet further consisting of, the administration of FOLFOX chemotherapy or an equivalent thereof, for a gastrointestinal cancer patient in need thereof, wherein the patient is more likely to experience longer overall survival following treatment comprising, or alternatively consisting essentially of, or yet further consisting of, determining the expression level of at least one gene of the group HIF1α or VEGFR2 in a suitable patient tissue or cell sample, wherein low HIF1α expression or low VEGFR2 expression selects the patient for said therapy.

Also provided are methods for selecting second line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely to experience longer overall survival comprising, or alternatively consisting essentially of, or yet further consisting of, determining the expression level of Glut1 in a suitable patient tissue or cell sample, wherein low Glut1 expression selects the patient for said therapy.

Treatment methods are also provided. For example methods for treating a gastrointestinal cancer patient in need thereof comprising, or alternatively consisting essentially of, or yet further consisting of, first line FOLFOX in combination with PTK/ZK chemotherapy or equivalent of each thereof, the method comprising, or alternatively consisting essentially of, or yet further consisting of, determining the expression level of at least one gene of the group LDHA, Glut1, or VEGFR1, in a suitable patient tissue or cell sample, and administering an effective amount of said treatment to a patient having high LDHA expression, high Glut1 expression, or high VEGFR1 expression of said respective gene, thereby treating the patient. Methods of determining an effective amount are known in the art and can be empirically determined by the treating physician.

Also provided are methods for treating a gastrointestinal cancer patient in need thereof comprising, or alternatively consisting essentially of, or yet further consisting of, second line FOLFOX in combination with PTK/ZK chemotherapy or equivalent of each thereof, the method comprising, or alternatively consisting essentially of, or yet further consisting of, determining the expression level of a HIF1α gene in a suitable patient tissue or cell sample, and administering an effective amount of said treatment to a patient having low HIF1α expression, thereby treating the patient. Methods of determining an effective amount are known in the art and can be empirically determined by the treating physician.

Also provided are methods for treating a gastrointestinal cancer patient in need thereof comprising, or alternatively consisting essentially of, or yet further consisting of, first line FOLFOX chemotherapy or an equivalent thereof, the method comprising, or alternatively consisting essentially of, or yet further consisting of, determining the expression level of at least one gene of the group HIF1α or VEGFR2 in a suitable patient tissue or cell sample, and administering an effective amount of said treatment to a patient having low HIF1α expression or low VEGFR2 expression, thereby treating the patient. Methods of determining an effective amount are known in the art and can be empirically determined by the treating physician.

In one aspect, the inventor has determined for certain cancer patients, age and gender correlate to overall survival following cancer treatment. Thus, this invention provides methods for identifying a metastatic colorectal cancer patient that may likely require more or most aggressive cancer treatment by correlating the gender, age and race of the patient to longer overall survival, wherein at least one patient of the group: a female patient greater than 44 years of age; or a male patient less than 76 years of age; or a female or male patient of any age of the race selected from the group consisting of Native American, African American or Asian, identifies said patient that may likely have worse or shorter overall survival than similarly situated patients.

In another aspect, this invention provides methods for identifying a metastatic colorectal cancer patient that may likely require less aggressive cancer treatment. This method requires correlating the gender, age and race of the patient to shorter overall survival, wherein at least one patient of the group: a female patient less than 45 years of age; or a male patient greater than 75 years of age; or a female or male patient of any age of the Hispanic or Caucasian race, identifies said patient as one that may likely have greater or longer overall survival than similarly situated patients.

In a further aspect, this invention are methods for identifying a metastatic gastric cancer patient that may likely require more or most aggressive cancer treatment by correlating the gender, age and race of the patient to longer overall survival, wherein at least one patient of the group: a female or male patient greater than 44 years of age; or a male patient of any age of the African American or Caucasian race, identifies said patient as one that may likely have worse or shorter overall survival than similarly situated patients.

In another aspect, this invention provides methods for identifying a metastatic gastric cancer patient that may likely require less aggressive cancer treatment, by correlating the gender, age and race of the patient to shorter overall survival, wherein at least one patient of the group: a male or female patient less than 45 years of age; or a male patient of the Asian race, identifies said patient as one that may likely have longer or greater overall survival than similarly situated patients.

In a separate aspect, this invention provides methods for identifying a gastric cancer patient that may likely have shorter time to tumor recurrence, comprising, or alternatively consisting essentially of, or yet further consisting of correlating the race of the patient with time to tumor recurrence, wherein at least one patient of the group a patient of the race Caucasian or a patient of the race Hispanic, identifies said patient as likely having shorter time to tumor recurrence.

This invention further provides methods for identifying a gastric cancer patient that may likely have longer time to tumor recurrence, comprising, or alternatively consisting essentially of, or yet further consisting of correlating the race of the patient with time to tumor recurrence, wherein a patient of the race Asian identifies said patient as likely having longer time to tumor recurrence.

In each of the above embodiments, this invention also provides treating said patient identified as requiring the appropriate therapy—more or less aggressive, as determined by the treating physician. Thus, this invention further provides correlating race as identified above and then further administering an effective amount of an appropriate therapy. For the purpose of illustration only, more aggressive and less aggressive therapies are described herein.

### BRIEF DESCRIPTION OF THE FIGURES

FIG. 1 shows hazard ratios of overall survival for males and females suffering from metastatic colorectal cancer correlated with age. Age groups are indicated.

FIG. 2 shows hazard ratios of overall survival for males and females suffering from metastatic colorectal cancer correlated with ethnicity. Ethnicity groups are indicated.

FIG. 3 shows a schematic depiction of tumor locations which correlate with gender in metastatic colorectal cancer. Tumor locations for male patients are indicated in dark gray, whereas tumor locations for female patients are indicated in light gray.

FIG. 4 shows ethnicity of a colorectal cancer patient correlates with overall survival. The different ethnic groups are indicated by arrows and in the figure legend.

FIG. 5 shows the Par-1 I-506D polymorphism predicts time to tumor recurrence in patients with surgically resected gastric cancer. The top curve indicates patients with the (Ins/Ins) genotype, the middle curve indicates (Ins/Del) genotype, and the bottom curve indicates the (Del/Del) genotype. The designation (N) represents the number of patients. The X-axis indicates the number of years since a patient was diagnosed with locally advanced gastric cancer and treated with surgical resection. The Y-axis indicates the estimated probability of a patient being recurrence free. The log-rank P value is equal to 0.016.

FIG. 6 shows the IL-8 T-251A polymorphism predicts time to tumor recurrence in patients with surgically resected gastric cancer. The top curve indicates patients with the (T/T) genotype, the middle curve indicates (T/A) genotype, and the bottom curve indicates the (A/A) genotype. The designation (n) represents the number of patients. The X-axis indicates the number of years since a patient was diagnosed with locally advanced gastric cancer and treated with surgical resection. The Y-axis indicates the estimated probability of a patient being recurrence free. The log-rank P value is equal to 0.007.

FIG. 7 shows the IL-1β (also identified herein as “IL-1b”) C+3954T polymorphism predicts time to tumor recurrence in patients with stage II colon cancer treated with 5-FU based adjuvant chemotherapy. The top curve indicates patients with the (C/C) genotype, the middle curve indicates (C/T) genotype, and the bottom curve indicates the (T/T) genotype. The designation (n) represents the number of patients. The X-axis indicates the number of years since a patient was diagnosed with stage II colon cancer and treated with 5-FU based adjuvant chemotherapy. The Y-axis indicates the estimated probability of a patient being recurrence free. The log-rank P value is <0.001.

FIG. 8 shows the IL-1 Ra VNTR polymorphism predicts time to tumor recurrence in patients with stage II colon cancer treated with 5-FU based adjuvant chemotherapy. The top curve indicates patients with the (2 repeat/2 repeat, also referred to herein as “Allele 2/Allele 2”) genotype, the middle curve indicates (4 repeat/4 repeat, also referred to herein as “Allele 1/Allele 1”) genotype, and the bottom curve indicates the (at least one allele with >4 repeats, also referred to herein as “Others/Others”) genotype. The designation (n) represents the number of patients. The X-axis indicates the number of years since a patient was diagnosed with stage II colon cancer and treated with 5-FU based adjuvant chemotherapy. The Y-axis indicates the estimated probability of a patient being recurrence free. The log-rank P value is equal to 0.006.

FIG. 9 shows the ICAM-1 codon K496E polymorphism predicts tumor response in patients with metastatic colorectal cancer treated with first line FOLFOX/BV or XELOX/BV. The percentage of patients showing complete response (CR), partial response (PR), stable disease (SD) or progressive disease (PD) is indicated within each patient population. The X-axis indicates the corresponding genotypes of the patients at the ICAM-1 codon K496E polymorphism. The Y-axis indicates the percentage of patients showing therapeutic response. The designation (n) represents the number of patients.

FIG. 10 shows the GRP78 (rs12009) polymorphism predicts tumor response in patients with metastatic colorectal cancer treated with first line FOLFOX/BV or XELOX/BV. The percentage of patients showing complete response (CR), partial response (PR), stable disease (SD) or progressive disease (PD) is indicated within each patient population. The X-axis indicates the corresponding genotypes of the patients at the GRP78 (rs12009) polymorphism. The Y-axis indicates the percentage of patients showing therapeutic response. The designation (n) represents the number of patients.

FIG. 11 shows the NFkB CA repeat polymorphism predicts progression free survival in patients with metastatic colorectal cancer treated with first line FOLFOX/BV or XELOX/BV. The top curve indicates patients with the (≧24/≧24) genotype, the middle curve indicates (<24/≧24) genotype, and the bottom curve indicates the (<24/<24) genotype. The designation (n) represents the number of patients. The X-axis indicates the number of month since the start of treatment with first line FOLFOX/BV or XELOX/BV. The Y-axis indicates the estimated probability of a patient's progression free survival.

FIG. 12 shows gene expression level of TS predicts progression-free survival for patients with stage II/III rectal cancer receiving three regimens of 5-fluorouracil and radiation. The top curve represents patients with low TS expression, the middle curve represents patients with high TS expression, and the bottom curve represents patients with medium (med) TS expression. The designation (N) represents the number of patients. The X-axis indicates the number of years patients were registered in the study. The Y-axis indicated the percent survival of the patient population. The P value is 0.02.

FIG. 13 shows gene expression level of TS predicts overall survival for patients with stage II/III rectal cancer receiving three regimens of 5-fluorouracil and radiation. The top curve represents patients with low TS expression, the middle curve represents patients with high TS expression, and the bottom curve represents patients with medium (med) TS expression. The designation (N) represents the number of patients. The X-axis indicates the number of years patients were registered in the study. The Y-axis indicated the percent survival of the patient population. The P value is 0.04.

FIG. 14 shows gene expression level of LDHA, Glut1 and VEGFR1 are predictive for tumor response in colorectal cancer patients receiving first line FOLFOX/PTK/ZK therapy (CONFIRM1 clinical trial). The X-axis represents the predictive genes, the number of patients with the designated expression level (N) and the threshold value used to determine high (>) and low (<) expression. The Y-axis represents the percentage of patients experiencing tumor response following treatment. The P values for the described genes are as follows: LDHA is 0.033, Glut1 is 0.045 and VEGFR1 is 0.012.

FIG. 15 shows gene expression level of HIF1α is predictive for tumor response in colorectal cancer patients receiving second line FOLFOX/PTK/ZK therapy (CONFIRM2 clinical trial). The X-axis represents the predictive gene, the number of patients with the designated expression level (N) and the threshold value used to determine high (>) and low (<) expression. The Y-axis represents the percentage of patients experiencing tumor response following treatment. The P value for HIF1α is 0.021.

FIG. 16 shows gene expression level of VEGFR1 or HIF1α are predictive for tumor response in first line (CONFIRM1) or second line (CONFIRM2) FOLFOX/PTK/ZK therapy, respectively. The designation (n) represents the number of patients. Threshold values used to determine high (>) and low (<) expression are indicated. Groups 2 and 3 show a higher percentage of patients experienced tumor response following their respective treatments.

FIG. 17 shows the gene expression level of VEGFR2 predicts progression free survival in patients with colorectal cancer treated with first line FOLFOX or FOLFOX/PTK/ZK therapy. The threshold value for determining high (>) and low (≦) along with the presence or absence of PTK/ZK is indicated. The designation (n) represents the number of patients. The X-axis indicates the number of month since the randomization of treatment. The Y-axis indicates the estimated probability of a patient's progression free survival. The p value for interaction between treatment and VEGFR2 expression is equal to 0.001.

FIG. 18 shows gene expression level of LDHA, or HIF1α and Glut1 are predictive for progression free survival for patients receiving first line (CONFIRM1) or second line (CONFIRM2) FOLFOX/PTK/ZK therapy, respectively. The designation (n) represents the number of patients. Threshold values used to determine high (≧) and low (<) expression are indicated. Groups 1, 3 and 4 show a lower hazard ratio (HR) for disease progression following their respective treatments.

FIG. 19 shows gene expression level of VEGFR2 or Glut1 are predictive for overall survival in first line (CONFIRM1) or second line (CONFIRM2) FOLFOX/PTK/ZK therapy, respectively. The designation (n) represents the number of patients. Threshold values used to determine high (≧) and low (<) expression are indicated. Groups 1, 3 and 4 show a lower hazard ratio (HR) for death following their respective treatments.

FIG. 20 shows gene expression level of VEGFR2 predicts survival in patients with colorectal cancer treated with first line FOLFOX/PTK/ZK therapy (CONFIRM1). The threshold value for determining high (>) and low (≦) is indicated. The designation (n) represents the number of patients. The X-axis indicates the number of month since the randomization of treatment. The Y-axis indicates the estimated probability of a patient's survival. The adjusted p value for VEGFR2 is 0.012.

FIG. 21a shows that metastatic colorectal adenocarcinoma patients from the CONFIRM-1 trial with higher intratumoral expression of LDHA had a significantly higher probability of progression-free survival following treatment with FOLFOX4 plus PTK/ZK than patients with lower LDHA gene expression levels (log rank p=0.004). The Kaplan-Meier curves show gene expression levels of lactate dehydrogenase A (LDHA) and progression-free survival in patients treated with FOLFOX4 plus PTK/ZK in CONFIRM-1.

FIG. 21b shows that metastatic colorectal adenocarcinoma patients from the CONFIRM-1 trial with higher intratumoral expression of VEGFR1 had a significantly higher probability of progression-free survival following treatment with FOLFOX4 plus PTK/ZK than patients with lower VEGFR1 gene expression levels (log rank p=0.023). The Kaplan-Meier curves show gene expression levels of vascular endothelial growth factor type-1 receptor (VEGFR1) and progression-free survival in patients treated with FOLFOX4 plus PTK/ZK in CONFIRM-1.

FIG. 21c shows that metastatic colorectal adenocarcinoma patients from the CONFIRM-2 trial with higher intratumoral expression of HIF1α had a significantly higher probability of progression-free survival following treatment with FOLFOX4 plus PTK/ZK than patients with lower HIF1α gene expression levels (log rank p=0.002). The Kaplan-Meier curves show gene expression levels of hypoxia-inducible factor type-1 alpha (HIF1α) and progression-free survival in patients treated with FOLFOX4 plus PTK/ZK in CONFIRM-2.

FIG. 22a shows a Recursive Partitioning Analysis of gene expression levels and clinical outcome in CONFIRM-1 and -2 with respect to tumor response. Numbers in squares represent number of responders (top line) and total number of patients (bottom line), and response rates are shown in parentheses. (HIF1α=hypoxia-inducible factor type-1 alpha; VEGFR1=vascular endothelial growth factor type-1 receptor)

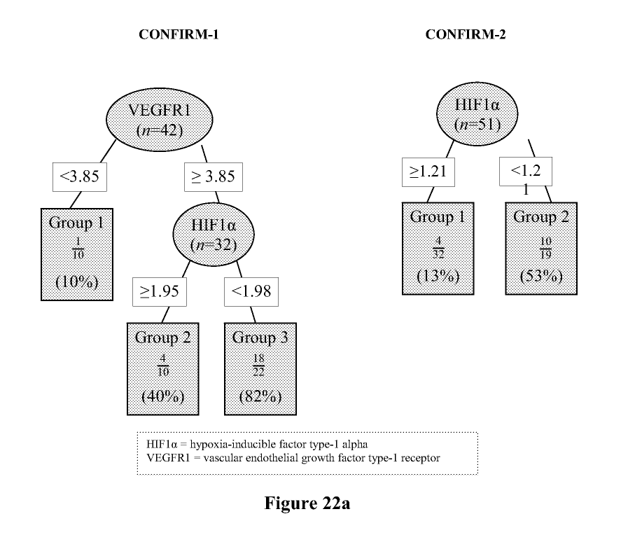

FIG. 22b shows a Recursive Partitioning Analysis of gene expression levels and clinical outcome in CONFIRM-1 and -2 with respect to Progression-free survival (PFS). Numbers in circles or squares denote number of patients. The hazard ratio (HR) indicates the risk of progressing when compared to the reference group (Group 1). Square boxes represent terminal nodes; circles represent the parent node and intermediate subgroups. (Glut-1=glucose transporter-1, HIF1α=hypoxia-inducible factor type-1 alpha, LDHA=lactate dehydrogenase A)

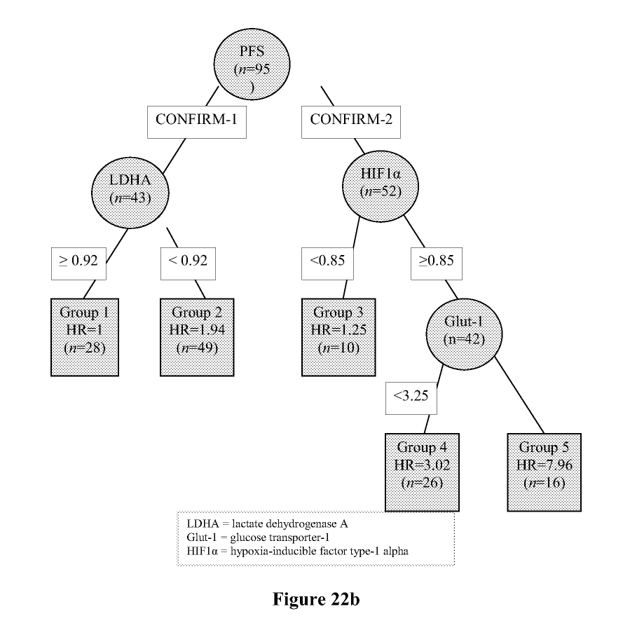

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S.C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### DEFINITIONS

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “identify” or “identifying” is to associate or affiliate a patient closely to a group or population of patients who likely experience the same or a similar clinical response to treatment.

Bevacizumab (BV) is sold under the trade name Avastin by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis.

Fluorouracil (5-FU) belongs to the family of therapy drugs call pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N[4[[(2-amino-5-formyl1,4,5,6,7,8hexahydro4oxo6-pteridinyl)methyl]amino]benzoyl], calcium salt (1:1).

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include without limitation cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

“FOLFOX” is an abbreviation for a type of combination therapy that is used to treat colorectal cancer. This therapy includes 5-FU, oxaliplatin and leucovorin. FOLFOX4 is a specific FOLFOX chemotherapy regimen known in the art and described herein. Information regarding these treatments are available on the National Cancer Institute's web site, cancer.gov, last accessed on Jan. 16, 2008.

“FOLFOX/BV” is an abbreviation for a type of combination therapy that is used to treat colorectal cancer. This therapy includes 5-FU, oxaliplatin, leucovorin and Bevacizumab. Furthermore, “XELOX/BV” is another combination therapy used to treat colorectal cancer, which includes the prodrug to 5-FU, known as Capecitabine (Xeloda) in combination with oxaliplatin and bevacizumab. Information regarding these treatments are available on the National Cancer Institute's web site, cancer.gov or from the National Comprehensive Cancer Network's web site, nccn.org, last accessed on May 27, 2008.

PTK/ZK is a “small” molecule tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. PTK/ZK is a targeted drug that blocks angiogenesis and lymphangiogenesis by inhibiting the activity of all known receptors that bind VEGF including VEGFR-1 (Flt-1), VEGFR-2 (KDR/Flk-1) and VEGFR-3 (Flt-4). The chemical names of PTK/ZK are 1-[4-Chloroanilino]-4-[4-pyridylmethyl]phthalazine Succinate or 1-Phthalazinamine, N-(4-chlorophenyl)-4-(4-pyridinylmethyl)-, butanedioate (1:1). Synonyms and analogs of PTK/ZK are known as Vatalanib, CGP79787D, PTK787/ZK 222584, CGP-79787, DE-00268, PTK-787, PTK-787A, VEGFR-TK inhibitor, ZK 222584 and ZK 232934.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

In one aspect, the therapy to be selected or administered to a patient is one that comprises, or alternatively consists essentially of, or yet further consists of a combination of pyrimidine based antimetabolite and an efficacy enhancing agent. One example of such therapy is know as 5-FU adjuvant therapy. “5-FU adjuvant therapy” refers to the combination of 5-FU with other treatments, such as without limitation, radiation, methyl-CCNU, Leucovorin, Oxaliplatin, irinotecin, mitomycin, cytarabine, levamisole. Specific treatment adjuvant regimens are known in the art as FOLFOX, FOLFOX4, MOF (semustine (methyl-CCNU), vincrisine (Oncovin) and 5-FU). For a review of these therapies see Beaven and Goldberg (2006) Oncology 20(5):461-460. An example of such is an effective amount of 5-FU and Leucovorin. Other chemotherapeutics can be added, e.g., Oxaliplatin.

“CONFIRM1” refers to a phase III clinical trail to compare treatment with 5-FU/oxaliplatin/leucovorin plus PTK/ZK versus 5-FU/oxaliplatin/leucovorin plus placebo in patients with colorectal cancer that has spread to other organs who were seeking first line chemotherapy treatment. Details regarding this clinical trial can be found at the website www.clinicaltrials.gov (last visited on Apr. 18, 2007).

“CONFIRM2” refers to a phase III clinical trail to compare treatment with 5-FU/oxaliplatin/leucovorin plus PTK/ZK versus 5-FU/oxaliplatin/leucovorin plus placebo in patients with colorectal cancer that has spread to other organs and whose disease has worsened after treatment with irinotecan. Details regarding this clinical trial can be found at the website www.clinicaltrials.gov (last visited on Apr. 18, 2007).

The phrase “first line” or “second line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

The term “adjuvant” chemotherapy refers to administration of a therapy or chemotherapeutic regimen to a patient after removal of a tumor by surgery. Adjuvant chemotherapy is typically given to minimize or prevent a possible cancer reoccurrence. Alternatively, “neoadjuvant” chemotherapy refers to administration of therapy or chemotherapeutic regimen before surgery, typically in an attempt to shrink the tumor prior to a surgical procedure to minimize the extent of tissue removed during the procedure.

In one aspect, the “biological equivalent” means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody. An example of an equivalent Bevacizumab antibody is one which binds to and inhibits the biologic activity of human vascular endothelial growth factor (VEGF).

In one aspect, the “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The phrase “aggressive cancer treatment” refers to the cancer treatment, combination of treatments, or a chemotherapy regimen that is effective for treating the target cancer tumor or cell, but is associated with or known to cause higher toxicity, more side effects or is known in the art to be less efficacious than another type of treatment for the specified cancer type. One of skill in the art will be able to determine if a cancer treatment, combination of treatments, or chemotherapy regimen is less, more, or most aggressive. For example, a less aggressive treatment for a colon cancer patient may include adjuvant chemotherapy comprising surgical resection of the primary tumor and a chemotherapy regimen comprising 5-FU, leucovorin and bevacizumab. While a more aggressive cancer treatment may include adjuvant chemotherapy comprising surgical resection and a chemotherapy regimen comprising FOLFOX and BV, whereas the most aggressive cancer treatment may include surgical resection and a chemotherapy regime comprising Irinotecan and Cetuximab.

The term “antigen” is well understood in the art and includes substances which are immunogenic. VEGF is an example of an antigen.

A “native” or “natural” or “wild-type” antigen is a polypeptide, protein or a fragment which contains an epitope and which has been isolated from a natural biological source. It also can specifically bind to an antigen receptor.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

If an antibody is used in combination with the above-noted chemotherapy or for diagnosis or as an alternative to the chemotherapy, the antibodies can be polyclonal or monoclonal and can be isolated from any suitable biological source, e.g., murine, rat, sheep and canine. Additional sources are identified infra.

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)₂ fragment, a bivalent fragment comprising two Fab fragments linked by a disulfide bridge at the hinge region; a Fd fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “antibody variant” is intended to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by heavy chain constant region genes.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “genetic marker” refers to an allelic variant of a polymorphic region of a gene of interest and/or the expression level of a gene of interest.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

The term “allelic variant of a polymorphic region of the gene of interest” refers to a region of the gene of interest having one of a plurality of nucleotide sequences found in that region of the gene in other individuals.

The term “genotype” refers to the specific allelic composition of an entire cell or a certain gene and in some aspects a specific polymorphism associated with that gene, whereas the term “phenotype’ refers to the detectable outward manifestations of a specific genotype.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “gene of interest” intends one or more genes selected from the group consisting of TS, HIF1α (also referred to herein as HIF-1α), LDHA, Glut-1, VEGF, VEGFR1, VEGFR2, PAR-1, ES, IL-8, IL-1β (also referred to herein as IL-1b), IL-1Ra, ICAM-1, GRP78, NFkB and K-RAS.

“Expression” as applied to a gene, refers to the differential production of the mRNA transcribed from the gene or the protein product encoded by the gene. A differentially expressed gene may be over expressed (high expression) or under expressed (low expression) as compared to the expression level of a normal or control cell, a given patient population or with an internal control gene (house keeping gene). In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample.

In one aspect of the invention, a “predetermined threshold level” or “threshold value” is used to categorize expression as high or low. As a non-limiting example of the invention, the threshold level of VEGF is a level of VEGF expression above which it has been found in tumors likely to be resistant to FOLFOX in combination with PTK/ZK chemotherapy. Expression levels below this threshold level are likely to be found in tumors sensitive to FOLFOX in combination with PTK/ZK chemotherapy. In another aspect of the invention, the expression level threshold for LDHA is 0.36 or 0.92; Glut1 is 1.5, 2.12, 3.25 or 3.28; VEGFR1 is 3.78 or 3.85, HIF1α is 0.85, 1.18 or 1.21; VEGFR2 is 1.76, 1.78 or 2.98. In one aspect of the invention, gene expression identified as a ratio above the threshold level is categorized as high expression, whereas a ratio below the threshold level is categorized as low expression. The gene expression threshold for determining TS high, medium or low expression is know in the art and examples of which are described in Shirota et al. (2001) J. Clin. Oncol. 19(23):4298-4304; Pullarkat et al. (2001) Pharmacogenomics J. 1(1):65-70; U.S. Pat. Nos. 7,049,059; 7,132,238; 6,573,052; and 6,602,670; and U.S. Publ. Nos.: 2006/0094012 and 2006/0115827.

In another aspect, the threshold level of a gene is a level of expression below which it has been found in tumors likely to be responsive, or alternatively, non-responsive to the same treatment for a defined cancer type.

The term “expressed” also refers to nucleotide sequences in a cell or tissue which are expressed where silent in a control cell or not expressed where expressed in a control cell.

In another aspect, “expression” level is determined by measuring the expression level of a gene of interest for a given patient population, determining the median expression level of that gene for the population, and comparing the expression level of the same gene for a single patient to the median expression level for the given patient population. For example, if the expression level of a gene of interest for the single patient is determined to be above the median expression level of the patient population, that patient is determined to have high expression of the gene of interest. Alternatively, if the expression level of a gene of interest for the single patient is determined to be below the median expression level of the patient population, that patient is determined to have low expression of the gene of interest.

A “internal control” or “house keeping” gene refers to any constitutively or globally expressed gene whose presence enables an assessment of the gene of interests expression level. Such an assessment comprises a determination of the overall constitutive level of gene transcription and a control for variation in sampling error. Examples of such genes include, but are not limited to, β-actin, the transferring receptor gene, GAPDH gene or equivalents thereof. In one aspect of the invention, the internal control gene is β-actin.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

A “blood cell” refers to any of the cells contained in blood. A blood cell is also referred to as an erythrocyte or leukocyte, or a blood corpuscle. Non-limiting examples of blood cells include white blood cells, red blood cells, and platelets.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” wherein X and Y are two different nucleotides, e.g., G/C for the IL-6 gene at position −174. If the allelic composition of a SNP locus is heterozygous, the genotype of the SNP locus will be identified as “X/X” wherein X identifies the nucleotide that is present at both alleles, e.g., G/G for IL-6 gene at position −174. In another aspect, a polymorphic locus harbors allelic variants of nucleotide sequences of different length. The genotype of the polymorphic locus will be identified with the length of the allelic variant, e.g., both alleles with <20 CA repeats at intron 1 of the EGFR gene. The genotype of the cell or tissue sample will be identified as a combination of genotypes of all polymorphic loci of interest, e.g. G/G for IL-6 gene at position −174 and both alleles with <20 CA repeats at intron 1 of the EGFR gene.

“Expression” as applied to a gene, refers to the production of the mRNA transcribed from the gene, or the protein product encoded by the gene. The expression level of a gene may be determined by measuring the amount of mRNA or protein in a cell or tissue sample. In one aspect, the expression level of a gene is represented by a relative level as compared to a housekeeping gene as an internal control. In another aspect, the expression level of a gene from one sample may be directly compared to the expression level of that gene from a different sample using an internal control to remove the sampling error.

An “internal control” or “house keeping” gene refers to any constitutively or globally expressed gene. Examples of such genes include, but are not limited to, β-actin, the transferring receptor gene, GAPDH gene or equivalents thereof. In one aspect of the invention, the internal control gene is β-actin.

“Overexpression” or “underexpression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

A “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present invention, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

Alternatively, a gene expression that is higher than the predetermined value is simply referred to as a “high expression”, or a gene expression that is lower than the predetermined value is simply referred to as a “low expression”.

Briefly and for the purpose of illustration only, one of skill in the art can determine a predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

The term “mismatches” refers to hybridized nucleic acid duplexes which are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicated the amount of a compound or agent administered or delivered to the patient which is most likely to result in the desired response to treatment. The amount is empirically determined by the patient's clinical parameters including, but not limited to the stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more more desirable clinical outcome as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same disease and receiving the same therapy but not possessing the characteristic. As defined herein, the patients is considered suitable for the therapy. In another such aspect, a patient possessing a characteristic may exhibit one or more more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

A “lymph node” refers to a rounded mass of lymphatic tissue that is surrounded by a capsule of connective tissue, which filter lymphatic fluid and stores white blood cells. Cancers described herein can spread to the lymphatic system and this spreading is used, in part, to determine the cancer stage. For example, if a cancer is “lymph node negative,” the cancer has not spread to the surrounding or nearby lymph nodes and thus the lymphatic system. Conversely, if the cancer has spread to the surrounding or nearby lymph nodes, the cancer is “lymph node positive.”

The term “whole blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

The term “hazard ratio” is a survival analysis in the effect of an explanatory variable on the hazard or risk of an event. In another aspect, “hazard ratio” is an estimate of relative risk, which is the risk of an event or development of a disease relative to treatment and in some aspects the expression levels of the gene of interest. Statistical methods for determining hazard ratio are well known in the art.

### DESCRIPTIVE EMBODIMENTS

Sex, Age and Ethnicity are Associated with Survival in Metastatic Colorectal Cancer

In one aspect, the inventor has determined for certain cancer patients, age and gender correlate to overall survival following cancer treatment. Thus, this invention provides methods for identifying a metastatic colorectal cancer patient that may likely require more or most aggressive cancer treatment by correlating the gender, age and race of the patient to longer overall survival, wherein at least one patient of the group: a female patient greater than 44 years of age; or a male patient less than 76 years of age; or a female or male patient of any age of the race selected from the group consisting of Native American, African American or Asian, identifies said patient that may likely have worse or shorter overall survival than similarly situated patients.

In another aspect, this invention provides methods for identifying a metastatic colorectal cancer patient that may likely require less aggressive cancer treatment. This method requires correlating the gender, age and race of the patient to shorter overall survival, wherein at least one patient of the group: a female patient less than 45 years of age; or a male patient greater than 75 years of age; or a female or male patient of any age of the Hispanic or Caucasian race, identifies said patient as one that may likely have greater or longer overall survival than similarly situated patients.

Age and Ethnicity Predict Overall Survival in Patient with Metastatic Gastric Cancer

In a further aspect, this invention are methods for identifying a metastatic gastric cancer patient that may likely require more or most aggressive cancer treatment by correlating the gender, age and race of the patient to longer overall survival, wherein at least one patient of the group: a female or male patient greater than 44 years of age; or a male patient of any age of the African American or Caucasian race, identifies said patient as one that may likely have worse or shorter overall survival than similarly situated patients.

In another aspect, this invention provides methods for identifying a metastatic gastric cancer patient that may likely require less aggressive cancer treatment, by correlating the gender, age and race of the patient to shorter overall survival, wherein at least one patient of the group: a male or female patient less than 45 years of age; or a male patient of the Asian race, identifies said patient as one that may likely have longer or greater overall survival than similarly situated patients.

In each of the above embodiments, this invention also provides treating said patient identified as requiring the appropriate therapy—more or less aggressive, as determined by the treating physician. Thus, this invention further provides correlating age, sex and race as identified above and then further administering an effective amount of an appropriate therapy. For the purpose of illustration only, more aggressive and less aggressive therapies are described herein. In another aspect of the invention, the above methods correlating age, sex and race with cancer treatment can be combined with the following methods for identifying, selecting, or treating a cancer patient that is likely to experience tumor recurrence, show responsiveness, experience longer or shorter overall survival or experience longer or shorter progression free survival following treatment.

Polymorphisms in PAR-1, ES and IL-8 Predict Tumor Recurrence

This invention provides methods for identifying a gastrointestinal cancer patient that is more likely to experience tumor recurrence following surgical resection of a tumor, comprising, or alternatively consisting essentially of, or yet further consisting of screening a suitable patient tissue or cell sample for one genotype of the group PAR-1 I-506D, ES G+4349A or IL-8 T-251A polymorphisms, wherein (ins/ins) for Par-1 I-506D; (A/A) for IL-8 T-251A; or (A/A) for ES G+4349A, respectively, identifies the patient as more likely to experience tumor recurrence following surgical resection of a tumor.

Also provided herein are methods for identifying a gastrointestinal cancer patient that is less likely to experience tumor recurrence following surgical resection of a tumor, comprising or alternatively consisting essentially of, or yet further consisting of, screening a suitable patient tissue or cell sample for sample for one genotype of the group PAR-1 I-506D, ES G+4349A or IL-8 T-251A polymorphisms, wherein (del/del or ins/del) for Par-1 I-506D; (T/T or T/A) for IL-8 T-251A; or (G/G or G/A) for ES G+4349A, respectively, identifies the patient as less likely to experience tumor recurrence following surgical resection of a tumor

In the above methods, the gastrointestinal cancer is a metastatic or non-metastatic cancer selected from rectal cancer colorectal cancer, colon cancer, gastric cancer or esophageal cancer.

In a further aspect, the patient sample for practicing these methods comprises, or alternatively consists essentially of, or yet further consists of, tissue or cells selected from non-metastatic tumor tissue, a non-metastatic tumor cell, metastatic tumor tissue, a metastatic tumor cell, peripheral blood lymphocytes or whole blood. In a further aspect, the patient sample comprises peripheral blood lymphocytes. In another aspect the patient sample can be normal tissue isolated adjacent to the tumor. In another aspect the patient sample can be normal tissue isolated distal to the tumor or any other normal tissue.

Although the methods are not limited by the means by which the genotype is determined, in one aspect the genotype is determined by a method comprising, or alternatively consisting essentially of, or yet further consisting of, hybrization or PCR. In a particular aspect, the genotype is determined by a method comprising, or alternatively consisting essentially of, or yet further consisting of, PCR-RFLP.

In a further aspect, the genotype is determined by a method comprising, or alternatively consisting essentially of, or yet further consisting of, contacting a suitable nucleic acid sample isolated from the patient sample with an array comprising a probe or primer that selectively hybridizes to a fragment of a respective gene of the group PAR-1 I-506D, ES G+4349A or IL-8 T-251A under conditions favoring the formation of nucleic acid hybridization pairs and detecting the presence of any pair so formed. Methods of detecting such pairs are known to the skilled artisan and non-limiting examples of such are described herein.

Thus, in one aspect of the above methods, the invention is a method for identifying a gastric cancer patient that is less likely to experience tumor recurrence following surgical resection of a tumor, comprising, or alternatively consisting essentially of, or yet further consisting of, screening peripheral blood lymphocytes from the patient for one genotype by a method comprising PCR-RFLP of the group PAR-1 I-506D, ES G+4349A or IL-8 T-251A polymorphisms, wherein (del/del or ins/del) for Par-1 I-506D; (T/T or T/A) for IL-8 T-251A; or (G/G or G/A) for ES G+4349A, respectively, identifies the patient as less likely to experience tumor recurrence following surgical resection of a tumor.

Polymorphisms in IL-1β and IL-1Ra Predict Tumor Recurrence

This invention also provides methods for identifying a stage II colon cancer patient that is more likely to show responsiveness to 5-FU based adjuvant chemotherapy regimen or equivalent thereof, comprising, or alternatively consisting essentially of, or yet further consisting of, screening a suitable cell or tissue sample for at least one genotype of IL-1β C+3954T, IL-1Ra VNTR or VEGF G-634C polymorphisms, wherein (C/C or C/T) for IL-1β C+3954T; (4 repeats/4 repeats or 2 repeats/2 repeats) for IL-1Ra VNTR or (G/G) for VEGF G-634C, respectively, identifies the patient as more likely to show responsive to said therapy.

Also provided are methods for identifying a stage II colon cancer patient that is more likely to experience tumor recurrence following 5-FU based adjuvant chemotherapy regimen or equivalent thereof, comprising, or alternatively consisting essentially of, or yet further consisting of, screening a suitable patient cell or tissue sample for at least one genotype of IL-1β C+3954T, IL-1Ra VNTR or VEGF G-634C, wherein (T/T) for IL-1β C+3954T; (at least one allele with >4 repeats) for IL-1Ra VNTR; or (C/C or C/G) for VEGF G-634C, respectively, identifies the patient as more likely to experience tumor recurrence following said therapy.

Yet further provided are methods for selecting a therapy comprising 5-FU based adjuvant chemotherapy regimen or equivalent thereof for a stage II colon cancer patient in need thereof, comprising, or alternatively consisting essentially of, or yet further consisting of, screening a suitable patient cell or tissue sample for the presence of a genotype (C/C or C/T) for IL-1β C+3954T; (4 repeats/4 repeats or 2 repeats/2 repeats) for IL-1Ra VNTR or (G/G) for VEGF G-634C, respectively, wherein the presence of said genotype selects said patient for said chemotherapy.

Also provided are methods for treating a stage II colon cancer patient selected for therapy comprising, or alternatively consisting essentially of, or yet further consisting of, administration of a 5-FU based adjuvant chemotherapy regimen or equivalent thereof, comprising, or alternatively consisting essentially of, or yet further consisting of screening a suitable cell or tissue sample for the presence of a genotype (C/C or C/T) for IL-1β C+3954T; (4 repeats/4 repeats or 2 repeats/2 repeats) for IL-1Ra VNTR; or (G/G) for VEGF G-634C, and administering an effective amount of said chemotherapy to a patient having a genotype identified above, thereby treating said patient.

In one aspect, tumor recurrence is measured by risk of tumor recurrence, time tot tumor recurrence or disease free survival after treatment with said therapy as compared to similarly situated patients.

In each of the above methods, the patient sample comprises, or alternatively consists essentially of, or yet further consists of, tissue or cells selected from non-metastatic tumor tissue, a non-metastatic tumor cell, metastatic tumor tissue, a metastatic tumor cell, peripheral blood lymphocytes or whole blood. In one aspect, the patient sample comprises or alternatively consists essentially of, or yet further consists of, a non-metastatic tumor cell or tissue. In a yet further aspect, the patient sample comprises, or alternatively consists essentially of, or yet further consists of peripheral blood lymphocytes. In another aspect the patient sample can be normal tissue isolated adjacent to the tumor. In another aspect the patient sample can be normal tissue isolated distal to the tumor or any other normal tissue.

Although the methods are not limited by the means by which the identify of the genotype is determined, in one aspect the genotype is determined by a method comprising, or alternatively consisting essentially of, or yet further consisting of hybridization or PCR. As a particular non-limiting example, the genotype is determined by a method comprising, or alternatively consisting essentially of, or yet further consisting of PCR-RFLP.

Also as a non-limiting example, the genotype is determined by a method comprising, or alternatively consisting essentially of, or yet further consisting of, contacting nucleic acids isolated from the patient sample with an array comprising a probe or primer that selectively hybridizes to a fragment of said respective gene under conditions favoring the formation of nucleic acid hybridization pairs and detecting the presence of any pair so formed.

In each of these methods, wherein the 5-FU based adjuvant chemotherapy comprises, or alternatively consists essentially of, or yet further consists of FOLFOX (5-FU, leucovorin and oxaliplatin); FOLFIRI (5-FU, leucovorin and irinotecan) or 5-FU and leucovorin.

Thus, in one aspect of the above methods, this invention provides methods for identifying a stage II colon cancer patient that is less likely to experience tumor recurrence following 5-FU based adjuvant chemotherapy regimen comprising, or alternatively consisting essentially of, or yet further consisting of, screening peripheral blood lymphocytes from the patient for at least one genotype by a method comprising PCR-RFLP of IL-1β C+3954T, IL-1Ra VNTR or VEGF G-634C polymorphisms, wherein (C/C or C/T) for IL-1β C+3954T; (4 repeats/4 repeats or 2 repeats/2 repeats) for IL-1Ra VNTR or (G/G) for VEGF G-634C, respectively, identifies the patient as less likely to experience tumor recurrence following said therapy.

Ethnicity is Associated with Recurrence in Patients with Resected Gastric Cancer

In a separate aspect, this invention provides methods for identifying a gastric cancer patient that may likely have shorter time to tumor recurrence, comprising, or alternatively consisting essentially of, or yet further consisting of correlating the race of the patient with time to tumor recurrence, wherein at least one patient of the group a patient of the race Caucasian or a patient of the race Hispanic, identifies said patient as likely having shorter time to tumor recurrence.

This invention further provides methods for identifying a gastric cancer patient that may likely have longer time to tumor recurrence, comprising, or alternatively consisting essentially of, or yet further consisting of correlating the race of the patient with time to tumor recurrence, wherein a patient of the race Asian identifies said patient as likely having longer time to tumor recurrence.

In each of the above embodiments, this invention also provides treating said patient identified as requiring the appropriate therapy—more or less aggressive, as determined by the treating physician. Thus, this invention further provides correlating race as identified above and then further administering an effective amount of an appropriate therapy. For the purpose of illustration only, more aggressive and less aggressive therapies are described herein. In another aspect of the invention, the above methods correlating ethnicity with cancer treatment can be combined with the herein described methods for identifying, selecting, or treating a cancer patient that is likely to experience tumor recurrence, show responsiveness, experience longer or shorter overall survival or experience longer or shorter progression free survival following treatment.

Polymorphism in ICAM, GRP-78 and NFkB Predicted Clinical Outcome

Methods for identifying a gastrointestinal cancer patient that is more likely to show responsiveness to first line FOLFOX/BV or first line XELOX/BV chemotherapy regimen or equivalent of each thereof is provided by screening a suitable patient cell or tissue sample for at least one genotype of the group of ICAM-1 codon K496E, GRP78 (rs12009), or NFkB CA repeat, wherein (C/C or C/T) for ICAM-1 codon K496E; (C/C or C/T) for GRP78 (rs12009); or (at least 1 allele with ≧24 CA repeats) for NFkB CA repeat, respectively, identifies the patient as more likely to show responsiveness to said therapy.

Also provided are methods for identifying a gastrointestinal cancer patient that is less likely to show responsiveness to first line FOLFOX/BV or first line XELOX/BV chemotherapy regimen or equivalent of each thereof, comprising, or alternatively consisting essentially of, or yet further, consisting of screening a suitable patient cell or tissue sample for at least one genotype of the group of ICAM-1 codon K496E, GRP78 (rs12009), or NFkB CA repeat, wherein (T/T) for ICAM-1 codon K496E; (T/T) for GRP78 (rs12009); or (two alleles with <24 CA repeats) for NFkB CA repeat, respectively, identifies the patient as less likely to show responsiveness to said therapy.

Further provided are methods for selecting a therapy comprising first line FOLFOX/BV or first line XELOX/BV chemotherapy regimen or equivalent of each thereof for a gastrointestinal patient in need thereof, comprising, or alternatively consisting essentially of, or yet further consisting of screening a suitable cell or tissue sample for at least one genotype of the group (C/C or C/T) for ICAM-1 codon K496E; (C/C or C/T) for GRP78 (rs12009); or (at least 1 allele with ≧24 CA repeats) for NFkB CA repeat, wherein the presence of at least one of said genotype selects the patient for said chemotherapy regimen.

Yet further provided are methods for treating a gastrointestinal cancer patient selected for therapy comprising, or alternatively consisting essentially of, or yet further consisting of, administration of a first line FOLFOX/BV or first line XELOX/BV chemotherapy regimen or equivalent of each thereof, comprising, or alternatively consisting essentially of, or yet further consisting of, screening a suitable patient cell or tissue sample for the presence of at least one genotype of the group: (C/C or C/T) for ICAM-1 codon K496E; (C/C or C/T) for GRP78 (rs12009); or (at least 1 allele with ≧24 CA repeats) for NFkB CA repeat, administering an effective amount of said chemotherapy to a patient having at least one genotype identified above, thereby treating said patient. Methods of determining an effective amount are known in the art and can be empirically determined by the treating physician.

For the above methods, likelihood of responsiveness is measured by at least one of the group complete response (CR), partial response (PR), stable disease (SD), progressive disease (PD) or progression free survival (PFS). In addition, the gastrointestinal cancer is a metastatic or non-metastatic cancer selected from the group of rectal cancer colorectal cancer, colon cancer, gastric cancer or esophageal cancer.

In each of the above methods, the patient sample comprises, or alternatively consists essentially of, or yet further consists of, tissue or cells selected from non-metastatic tumor tissue, a non-metastatic tumor cell, metastatic tumor tissue, a metastatic tumor cell or peripheral blood lymphocytes. In one aspect, the patient sample comprises or alternatively consists essentially of, or yet further consists of, a non-metastatic tumor cell or tissue. In a yet further aspect, the patient sample comprises, or alternatively consists essentially of, or yet further consists of peripheral blood lymphocytes. In another aspect the patient sample can be normal tissue isolated adjacent to the tumor. In another aspect the patient sample can be normal tissue isolated distal to the tumor or any other normal tissue.

Although the methods are not limited by the means by which the identify of the genotype is determined, in one aspect the genotype is determined by a method comprising, or alternatively consisting essentially of, or yet further consisting of hybridization or PCR. As a particular non-limiting example, the genotype is determined by a method comprising, or alternatively consisting essentially of, or yet further consisting of PCR-RFLP.

Also as a non-limiting example, the genotype is determined by a method comprising, or alternatively consisting essentially of, or yet further consisting of, contacting nucleic acids isolated from the patient sample with an array comprising a probe or primer that selectively hybridizes to a fragment of said respective gene under conditions favoring the formation of nucleic acid hybridization pairs and detecting the presence of any pair so formed.

Thus, in one aspect of the above methods, the invention is a method for identifying a metastatic colon cancer patient that is more likely to show responsiveness to first line FOLFOX/BV or first line XELOX/BV chemotherapy regimen comprising, or alternatively consisting essentially of, or alternatively consisting of, screening peripheral blood lymphocytes from the patient for at least one genotype by a method comprising PCR-RFLP of the group of ICAM-1 codon K496E, GRP78 (rs12009), or NFkB CA repeat, wherein (C/C or C/T) for ICAM-1 codon K496E; (C/C or C/T) for GRP78 (rs12009); or (at least 1 allele with ≧24 CA repeats) for NFkB CA repeat, identifies the patient as more likely to show responsive to said therapy.

K-RAS Mutation Status Predicts Clinical Outcome

This invention also provides methods for identifying a gastrointestinal cancer patient that is more likely to show responsiveness to FOLFOX/BV or XELOX/BV chemotherapy regimen or equivalent thereof, comprising, or alternatively consisting essentially of, or yet further consisting of, screening a suitable patient cell or tissue sample for at least one genotype of K-RAS codon 12 or K-RAS codon 13, wherein a wild type K-RAS codon 12 (GGT) and a wild type K-RAS codon 13 (GGC), respectively, of the K-RAS gene identifies the patient as more likely to show responsive to said therapy.

Further provided are methods for identifying a gastrointestinal cancer patient that is less likely to show responsiveness to FOLFOX/BV or XELOX/BV chemotherapy regimen or equivalent thereof, comprising, or alternatively consisting essentially of, or yet further consisting of, screening a suitable patient cell or tissue sample for at least one genotype of K-RAS codon 12 or K-RAS codon 13, wherein a mutation in K-RAS codon 12 or K-RAS codon 13 of the K-RAS gene, respectively, identifies the patient as less likely to show responsive to said therapy.

Also provided are methods for selecting a therapy comprising FOLFOX/BV or XELOX/BV chemotherapy regimen or equivalent thereof for a gastrointestinal cancer patient in need thereof, comprising, or alternatively consisting essentially of, or yet further consisting of, screening a suitable patient cell or tissue sample for the presence of a wild type K-RAS codon 12 (GGT) and a wild type K-RAS codon 13 (GGC) genotype of the K-RAS gene selects said patient for said chemotherapy.

Yet further are methods for treating a gastrointestinal cancer patient selected for therapy comprising, or alternatively consisting essentially of, or yet further consisting of, administration of a FOLFOX/BV or XELOX/BV chemotherapy regimen or equivalent thereof, comprising, or alternatively consisting essentially of, or yet further consisting of, screening a suitable patient cell or tissue sample for the presence of a wild type K-RAS codon 12 (GGT) and a wild type K-RAS codon 13 (GGC) genotype of the K-RAS gene; and administering an effective amount of said chemotherapy to a patient having a genotype identified in step a, thereby treating said patient. Methods of determining an effective amount are known in the art and can be empirically determined by the treating physician.

In one aspect for the above methods, likelihood of responsiveness is measured by progression free survival.

Also for the above methods, the gastrointestinal cancer is a metastatic or non-metastatic cancer selected from the group of rectal cancer colorectal cancer, colon cancer, gastric cancer or esophageal cancer.

In each of the above methods, the patient sample comprises, or alternatively consists essentially of, or yet further consists of, tissue or cells selected from non-metastatic tumor tissue, a non-metastatic tumor cell, metastatic tumor tissue, a metastatic tumor cell or peripheral blood lymphocytes. In one aspect, the patient sample comprises or alternatively consists essentially of, or yet further consists of, a non-metastatic tumor cell or tissue. In a yet further aspect, the patient sample comprises, or alternatively consists essentially of, or yet further consists of peripheral blood lymphocytes. In another aspect the patient sample can be normal tissue isolated adjacent to the tumor. In another aspect the patient sample can be normal tissue isolated distal to the tumor or any other normal tissue.

Although the methods are not limited by the means by which the identify of the genotype is determined, in one aspect the genotype is determined by a method comprising, or alternatively consisting essentially of, or yet further consisting of hybridization, PCR or direct sequencing. As a particular non-limiting example, the genotype is determined by a method comprising, or alternatively consisting essentially of, or yet further consisting of PCR-RFLP.

Also as a non-limiting example, the genotype is determined by a method comprising, or alternatively consisting essentially of, or yet further consisting of, contacting nucleic acids isolated from the patient sample with an array comprising a probe or primer that selectively hybridizes to a fragment of said respective gene under conditions favoring the formation of nucleic acid hybridization pairs and detecting the presence of any pair so formed.

Thus, in one aspect of the above methods, this invention provides a method for identifying a metastatic colorectal cancer patient that is more likely to experience longer progression free survival following FOLFOX/BV or XELOX/BV chemotherapy regimen, comprising, or alternatively consisting essentially of, or yet further consisting of, screening peripheral blood lymphocytes for at least one genotype by a method comprising PCR or direct sequencing for K-RAS codon 12 or K-RAS codon 13, wherein a wild type K-RAS codon 12 (GGT) and a wild type K-RAS codon 13 (GGC) of the K-RAS gene identifies the patient as more likely to experience longer progression free survival following said therapy.

Gene Expression of TS in Tumor Tissue Predicts Overall Survival and Progression Free Survival

This invention also provides methods for identifying a stage II or stage III rectal cancer patient that is more likely to experience longer relative overall survival or progression fee survival following treatment comprising, or alternatively consisting essentially of, or yet further consisting of, the administration of 5-FU or an equivalent thereof and pelvic radiation, comprising, or alternatively consisting essentially of, or yet further consisting of, screening a suitable patient tissue or cell sample for the expression level of the thymidylate synthase gene, wherein low expression of the gene identifies the patient as more likely to experience longer relative overall survival or progression fee survival following said therapy.

Further provided are methods for identifying a stage II or stage III rectal cancer patient that is more likely to experience shorter relative overall survival or progression fee survival following treatment comprising, or alternatively consisting essentially of, or yet further consisting of, the administration of 5-FU or an equivalent thereof and pelvic radiation, comprising, or alternatively consisting essentially of, or yet further consisting of, screening a suitable patient tissue or cell sample for the expression level of the thymidylate synthase gene, wherein high or medium expression of the gene identifies the patient as more likely to experience shorter relative overall survival or progression fee survival following said therapy.

Yet further provided is a method for selecting therapy comprising, or alternatively consisting essentially of, or yet further consisting of, the administration of 5-FU or an equivalent thereof and pelvic radiation to a stage II or stage III rectal cancer patient in need thereof, comprising, or alternatively consisting essentially of, or yet further consisting of, determining the expression level of the thymidylate synthase gene in a suitable patient tissue or cell sample, wherein low expression of said gene selects the patient for said therapy.

Also provided are methods for treating a stage II or stage III rectal cancer patient selected for treatment comprising administration of an effective amount of 5-FU or an equivalent thereof and pelvic radiation, the method comprising, or alternatively consisting essentially of, or yet further consisting of, determining the expression level of the thymidylate synthase gene in a suitable patient tissue or cell sample, administering an effective amount of said treatment to a patient having low expression of said gene, thereby treating the patient.

In each of the above methods, wherein the patient sample comprises, or alternatively consists essentially of, or yet further consists of tumor cells or tumor tissue.

Although the methods are not limited by the means by which gene expression is determined, in one aspect the expression level of the gene is determined by a method comprising, or alternatively consisting essentially of, or yet further consisting of, one or more of hybrization, PCR, or protein expression analysis. In a particular aspect, the expression level of the gene is determined by a method comprising, or alternatively consisting essentially of, or yet further consisting of, real-time fluorescent based PCR.

Thus, in one aspect of the above methods, this invention provides a method for identifying a stage II or stage III rectal cancer patient that is more likely to experience longer relative overall survival or progression fee survival following treatment comprising, or alternatively consisting essentially of, or yet further consisting of, the administration of 5-FU or an equivalent thereof and pelvic radiation, comprising, or alternatively consisting essentially of, or yet further consisting of, screening a tumor tissue from the patient for the expression level of the thymidylate synthase gene by fluorescence-based real-time PCR, wherein low expression of the gene identifies the patient as more likely to experience longer relative overall survival or progression fee survival following said therapy.

Intratumoral Expression of Genes Involved in Angiogenesis and HIF1 Pathway Predict Outcome

Also provided are methods for identifying a gastrointestinal cancer patient that is more likely responsive to therapy comprising, or alternatively consisting essentially of, or yet further consisting of, first line FOLFOX in combination with PTK/ZK chemotherapy or equivalent of each thereof, comprising, or alternatively consisting essentially of, or yet further consisting of, determining the expression level of at least one gene of the group LDHA, Glut1, or VEGFR1 in a suitable tissue or cell sample, wherein high LDHA expression, high Glut1 expression, or high VEGFR1 expression, respectively, identifies the patient that is more likely responsive to said therapy.

Further provided are methods for identifying a gastrointestinal cancer patient that is more likely responsive to therapy comprising, or alternatively consisting essentially of, or yet further consisting of, second line FOLFOX in combination with PTK/ZK chemotherapy or equivalent of each thereof, comprising, or alternatively consisting essentially of, or yet further consisting of, determining the expression level of HIF1α in a suitable patient tissue or cell sample, wherein low HIF1α expression identifies the patient that is more likely responsive to said therapy.

Yet further provided are methods for identifying a gastrointestinal cancer patient that is more likely to have progression free survival following therapy comprising, or alternatively consisting essentially of, or yet further consisting of, first line FOLFOX in combination with PTK/ZK chemotherapy or equivalent of each thereof, comprising, or alternatively consisting essentially of, or yet further consisting of, determining the expression level of at least one gene of the group VEGFR1 or LDHA in a suitable patient tissue or cell sample, wherein high VEGFR1 expression or high LDHA expression, respectively, identifies the patient that is more likely to have progression free survival following said therapy.

Also provided are methods for identifying a gastrointestinal cancer patient that is more likely to have progression free survival following therapy comprising, or alternatively consisting essentially of, or yet further consisting of, second line FOLFOX in combination with PTK/ZK chemotherapy or equivalent of each thereof, comprising, or alternatively consisting essentially of, or yet further consisting of, determining the expression level a HIF1α gene in a suitable tissue or cell sample, wherein low HIFα expression identifies the patient that is more likely to have progression free survival following said therapy.

Also provided are methods for identifying a gastrointestinal cancer patient that is more likely to have longer overall survival following therapy comprising, or alternatively consisting essentially of, or yet further consisting of, first line FOLFOX chemotherapy or an equivalent thereof, comprising, or alternatively consisting essentially of, or yet further consisting of, determining the expression level of at least one gene of the group HIF1α or VEGFR2 in a suitable patient tissue or cell sample, wherein low HIF1α expression or low VEGFR2 expression identifies the patient that is more likely to have longer overall survival following said therapy.

Alternatively, methods for identifying a gastrointestinal cancer patient that is more likely to have longer overall survival following therapy comprising second line FOLFOX in combination with PTK/ZK chemotherapy or equivalent of each thereof, comprising, or alternatively consisting essentially of, or yet further consisting of, determining the expression level of Glut1 in a suitable patient tissue or cell sample, wherein low Glut1 expression identifies the patient that is more likely to have longer overall survival following said therapy.

Also provided are method for selecting first line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely responsive to said therapy, comprising, or alternatively consisting essentially of, or yet further consisting of, determining the expression level of at least one gene of the group LDHA, Glut1 or VEGFR1 in a suitable patient tissue or cell sample, wherein high LDHA expression, high Glut1 expression, or high VEGFR1 expression, respectively, selects the patient for said therapy.

Also provided are methods for selecting second line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely responsive to said therapy, comprising, or alternatively consisting essentially of, or yet further consisting of, determining the expression level of HIF1α in a suitable patient tissue or cell sample, wherein low HIF1 expression selects the patient for said therapy.

Yet further are provided methods for selecting first line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely to experience longer progression free survival, comprising, or alternatively consisting essentially of, or yet further consisting of, determining the expression level of at least one gene of the group VEGFR1 or LDHA in a suitable patient tissue or cell sample, wherein high VEGFR1 expression or high LDHA expression, respectively, selects the patient for said therapy.

This invention also provides methods for selecting second line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely to experience longer progression free survival comprising, or alternatively consisting essentially of, or yet further consisting of, determining the expression level of a HIF1α gene in a suitable patient tissue or cell sample, wherein low HIF1α expression selects the patient for said therapy.

Also provided are methods for selecting first line therapy comprising, or alternatively consisting essentially of, or yet further consisting of, the administration of FOLFOX chemotherapy or an equivalent thereof, for a gastrointestinal cancer patient in need thereof, wherein the patient is more likely to experience longer overall survival following treatment comprising, or alternatively consisting essentially of, or yet further consisting of, determining the expression level of at least one gene of the group HIF1α or VEGFR2 in a suitable patient tissue or cell sample, wherein low HIF1α expression or low VEGFR2 expression selects the patient for said therapy.

Also provided are methods for selecting second line therapy comprising, or alternatively consisting essentially of, or yet further consisting of, the administration of FOLFOX in combination with PTK/ZK chemotherapy or equivalent of each thereof, for a gastrointestinal cancer patient in need thereof, wherein the patient is more likely to experience longer overall survival comprising, or alternatively consisting essentially of, or yet further consisting of, determining the expression level of Glut1 in a suitable patient tissue or cell sample, wherein low Glut1 expression selects the patient for said therapy.

Treatment methods are also provided. For example methods for treating a gastrointestinal cancer patient in need thereof comprising, or alternatively consisting essentially of, or yet further consisting of, first line FOLFOX in combination with PTK/ZK chemotherapy or equivalent of each thereof, the method comprising, or alternatively consisting essentially of, or yet further consisting of, determining the expression level of at least one gene of the group LDHA, Glut1, or VEGFR1, in a suitable patient tissue or cell sample, and administering an effective amount of said treatment to a patient having high LDHA expression, high Glut1 expression, or high VEGFR1 expression of said respective gene, thereby treating the patient. Methods of determining an effective amount are known in the art and can be empirically determined by the treating physician.

Also provided are methods for treating a gastrointestinal cancer patient in need thereof comprising, or alternatively consisting essentially of, or yet further consisting of, second line FOLFOX in combination with PTK/ZK chemotherapy or equivalent of each thereof, the method comprising, or alternatively consisting essentially of, or yet further consisting of, determining the expression level of a HIF1α gene in a suitable patient tissue or cell sample, and administering an effective amount of said treatment to a patient having low HIF1α expression, thereby treating the patient. Methods of determining an effective amount are known in the art and can be empirically determined by the treating physician.

Also provided are methods for treating a gastrointestinal cancer patient in need thereof comprising, or alternatively consisting essentially of, or yet further consisting of, first line FOLFOX chemotherapy or an equivalent thereof, the method comprising, or alternatively consisting essentially of, or yet further consisting of, determining the expression level of at least one gene of the group HIF1α or VEGFR2 in a suitable patient tissue or cell sample, and administering an effective amount of said treatment to a patient having low HIF1α expression or low VEGFR2 expression, thereby treating the patient. Methods of determining an effective amount are known in the art and can be empirically determined by the treating physician.

For these treatment methods, the gastrointestinal cancer is a metastatic or non-metastatic cancer selected from the group consisting of metastatic or non-metastatic rectal cancer, metastatic or non-metastatic colon cancer, metastatic or non-metastatic colorectal cancer, gastric cancer and esophageal cancer.

In each of the above methods, the patient sample comprises, or alternatively consists essentially of, or yet further consists of, tissue or cells selected from non-metastatic tumor tissue, a non-metastatic tumor cell, metastatic tumor tissue, a metastatic tumor cell or peripheral blood lymphocytes. In one aspect, the patient sample comprises or alternatively consists essentially of, or yet further consists of, a non-metastatic tumor cell or tissue.

Although the methods are not limited by the means by which the identify of the genotype is determined, in one aspect the genotype is determined by a method comprising, or alternatively consisting essentially of, or yet further consisting of hybridization or PCR. As a particular non-limiting example, the genotype is determined by a method comprising, or alternatively consisting essentially of, or yet further consisting of quantitative real time PCR.

Also as a non-limiting example, the genotype is determined by a method comprising, or alternatively consisting essentially of, or yet further consisting of, contacting nucleic acids isolated from the patient sample with an array comprising a probe or primer that selectively hybridizes to a fragment of said respective gene under conditions favoring the formation of nucleic acid hybridization pairs and detecting the presence of any pair so formed.

Thus, in one aspect of the above methods, the invention provides a method for identifying a metastatic colorectal cancer patient that is more likely responsive to therapy comprising, or alternatively consisting essentially of, or yet further consisting of, first line FOLFOX in combination with PTK/ZK chemotherapy, comprising, or alternatively consisting essentially of, or yet further consisting of, determining the expression level of at least one gene of the group LDHA, Glut1, or VEGFR1 in a suitable tissue or cell sample, wherein high LDHA expression, high Glut1 expression, or high VEGFR1 expression, respectively, identifies the patient that is more likely responsive to said therapy.

Further provided are methods for identifying a metastatic colorectal cancer patient that is more likely responsive to therapy comprising, or alternatively consisting essentially of, or yet further consisting of, second line FOLFOX in combination with PTK/ZK chemotherapy, comprising, or alternatively consisting essentially of, or yet further consisting of, determining the expression level of HIF1α in a suitable patient tissue or cell sample, wherein low HIF1α expression identifies the patient that is more likely responsive to said therapy.

Yet further provided is a method for identifying a metastatic colorectal cancer patient that is more likely to have progression free survival following therapy comprising, or alternatively consisting essentially of, or yet further consisting of, first line FOLFOX in combination with PTK/ZK chemotherapy, comprising, or alternatively consisting essentially of, or yet further consisting of, determining the expression level of at least one gene of the group VEGFR1 or LDHA in a suitable patient tissue or cell sample, wherein high VEGFR1 expression or high LDHA expression, respectively, identifies the patient that is more likely to have progression free survival following said therapy.

Also provided is a method for identifying a metastatic colorectal cancer patient that is more likely to have progression free survival following therapy comprising, or alternatively consisting essentially of, or yet further consisting of, second line FOLFOX in combination with PTK/ZK chemotherapy, comprising, or alternatively consisting essentially of, or yet further consisting of, determining the expression level of a HIF1α gene in a suitable tissue or cell sample, wherein low HIFα expression identifies the patient that is more likely to have progression free survival following said therapy.

Also provided is a method for identifying a metastatic colorectal cancer patient that is more likely to have longer overall survival following therapy comprising, or alternatively consisting essentially of, or yet further consisting of, first line FOLFOX in combination with PTK/ZK chemotherapy, comprising, or alternatively consisting essentially of, or yet further consisting of, determining the expression level of at least one gene of the group HIF1α or VEGFR2 in a suitable patient tissue or cell sample, wherein low HIF1α expression or low VEGFR2 expression identifies the patient that is more likely to have longer overall survival following said therapy.

Alternatively, a method for identifying a metastatic colorectal cancer patient that is more likely to have longer overall survival following therapy comprising second line FOLFOX in combination with PTK/ZK chemotherapy, comprising, or alternatively consisting essentially of, or yet further consisting of, determining the expression level of Glut1 in a suitable patient tissue or cell sample, wherein low Glut1 expression identifies the patient that is more likely to have longer overall survival following said therapy.

In one aspect of the above methods, the polymorphism of interest is present in a suitable patient cell or tissue sample. In one aspect the patient sample can be tumor tissue. In another aspect the patient sample can be normal tissue isolated adjacent to the tumor. In another aspect the patient sample can be a normal cell corresponding to the tumor tissue type. In a further aspect, the patient sample is any tissue of the patient, and can include peripheral blood lymphocytes or whole blood.

The methods are useful in the assistance of an animal, a mammal or yet further a human patient. For the purpose of illustration only, a mammal includes but is not limited to a simian, a murine, a bovine, an equine, a porcine or an ovine.

Diagnostic Methods

The invention further provides diagnostic methods, which are based, at least in part, on determination of the identity of the polymorphic region or the gene expression level of the genes identified herein.

Polymorphic Region

For example, information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations or additional base pair repeats can be accomplished by molecular cloning of the specified allele and subsequent sequencing of that allele using techniques known in the art, in some aspects, after isolation of a suitable nucleic acid sample using methods known in the art. Alternatively, the gene sequences can be amplified directly from a genomic DNA preparation from the tumor tissue using PCR, and the sequence composition is determined from the amplified product. As described more fully below, numerous methods are available for isolating and analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci, USA 74:560) or Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc. Natl. Acad. Sci USA 86:2766; Cotton (1993) Mutat. Res. 285:125-144 and Hayashi (1992) Genet Anal Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C. et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C. et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Often a solid phase support is used as a support capable of binding of a primer, probe, polynucleotide, an antigen or an antibody. Well-known supports include glass, polystyrene, polypropylene, polyethylene, dextran, nylon, amylases, natural and modified celluloses, polyacrylamides, gabbros, and magnetite. The nature of the support can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable supports for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject is likely responsive to the therapy as described herein or has or is at risk of developing disease such as colorectal cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Fetal nucleic acid samples can be obtained from maternal blood as described in International Patent Application No. WO91/07660 to Bianchi. Alternatively, amniocytes or chorionic villi can be obtained for performing prenatal testing.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

### Gene Expression Levels

The invention further provides diagnostic methods, which are based, at least in part, on determination of the expression level of a gene identified herein.

For example, information obtained using the diagnostic assays described herein is useful for determining if a subject will likely, or more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the gene expression levels of a particular gene in an individual (the genetic profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

In some aspects, the methods of the present invention require determining expression level of the gene of interest identified herein. These methods are not limited by the technique that is used to identify the expression level of the gene of interest. Methods for measuring gene expression are well known in the art and include, but are not limited to, immunological assays, nuclease protection assays, northern blots, in situ hybridization, reverse transcriptase Polymerase Chain Reaction (RT-PCR), Real-Time Polymerase Chain Reaction, expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli et al., (1998) J. Cell. Biochem. 72 (S30-31): 286-296; Galante et al., (2007) Bioinformatics, Advance Access (Feb. 3, 2007).

SAGE, MPSS, and SBS are non-array based assays that determine the expression level of genes by measuring the frequency of sequence tags derived from polyadenylated transcripts. SAGE allows for the analysis of overall gene expression patterns with digital analysis. SAGE does not require a preexisting clone and can used to identify and quantitate new genes as well as known genes. Velculescu et al., (1995) Science 270(5235):484-487; Velculescu (1997) Cell 88(2):243-251.

MPSS technology allows for analyses of the expression level of virtually all genes in a sample by counting the number of individual mRNA molecules produced from each gene.

As with SAGE, MPSS does not require that genes be identified and characterized prior to conducting an experiment. MPSS has a sensitivity that allows for detection of a few molecules of mRNA per cell. Brenner et al. (2000) Nat. Biotechnol. 18:630-634; Reinartz et al., (2002) Brief Funct. Genomic Proteomic 1: 95-104.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (Boon et al., (2002) PNAS 99:11287-92), SAGEmap (Lash et al., (2000) Genome Res 10:1051-1060), and Automatic Correspondence of Tags and Genes (ACTG) (Galante (2007), supra). The data can also be analyzed using databases constructed using in house computers (Blackshaw et al. (2004) PLoS Biol, 2:E247; Silva et al. (2004) Nucleic Acids Res 32:6104-6110)).

Over or under expression of a gene, in some cases, is correlated with a genomic polymorphism. The polymorphism can be present in a open reading frame (coded) region of the gene, in a “silent” region of the gene, in the promoter region, or in the 3′ untranslated region of the transcript. Methods for determining polymorphisms are well known in the art.

In other detection methods, it is necessary to first amplify at least a portion of the gene of interest prior to identifying the expression level. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

Antibodies directed against wild type or mutant peptides encoded by the gene of interest may also be used in determining gene expression levels for disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al., (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

Often a solid phase support is used as a support capable of binding a primer, probe, polynucleotide, an antigen or an antibody. Well-known supports include glass, polystyrene, polypropylene, polyethylene, dextran, nylon, amylases, natural and modified celluloses, polyacrylamides, gabbros, and magnetite. The nature of the support can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable supports for binding antibody or antigen, or will be able to ascertain the same by use of routine experimentation.

Moreover, it will be understood that any of the above methods for detecting alterations in gene expression levels can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject is likely responsive to the therapy as described herein or has or is at risk of developing disease such as colorectal cancer.

Sample nucleic acid for use in the above-described diagnostic and prognostic methods can be obtained from any cell type or tissue of a subject. For example, a subject's bodily fluid (e.g. blood) can be obtained by known techniques (e.g., venipuncture). Alternatively, nucleic acid tests can be performed on dry samples (e.g., hair or skin) Fetal nucleic acid samples can be obtained from maternal blood as described in International Patent Publ. No. WO 1991/007660 to Bianchi. Alternatively, amniocytes or chorionic villi can be obtained for performing prenatal testing.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) “PCR In Situ Hybridization: Protocols And Applications”, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

The invention described herein also relates to methods and compositions for determining and identifying the gene expression levels of the gene of interest. This information is useful to diagnose and prognose disease progression as well as select the most effective treatment among treatment options. Probes can be used to directly determine the gene expression levels in the sample or can be used simultaneously with or subsequent to amplification. The term “probes” includes naturally occurring or recombinant single- or double-stranded nucleic acids or chemically synthesized nucleic acids. They may be labeled by nick translation, Klenow fill-in reaction, PCR or other methods known in the art. Probes of the present invention, their preparation and/or labeling are described in Sambrook et al. (2001) supra. A probe can be a polynucleotide of any length suitable for selective hybridization to a nucleic acid of the gene of interest. Length of the probe used will depend, in part, on the nature of the assay used and the hybridization conditions employed.

Generally, any suitable oligonucleotide pairs that flank or hybridize to a gene of interest may be used to carry out the method of the invention. The invention provides specific oligonucleotide primers and probes that are particularly accurate in assessing the polymorphic region or expression levels of the genes of interest. However, other primers and/or probes are described in the art and are suitable for determining the polymorphic region or expression level of the genes of interest.

In one embodiment, it is necessary to first amplify at least a portion of the gene of interest prior to identifying the polymorphic region or level of gene expression of the gene of interest in a sample. Amplification can be performed, e.g., by PCR and/or LCR, according to methods known in the art. Various non-limiting examples of PCR include the herein described methods.

Allele-specific PCR is a diagnostic or cloning technique is used to identify or utilize single-nucleotide polymorphisms (SNPs). It requires prior knowledge of a DNA sequence, including differences between alleles, and uses primers whose 3′ ends encompass the SNP. PCR amplification under stringent conditions is much less efficient in the presence of a mismatch between template and primer, so successful amplification with an SNP-specific primer signals presence of the specific SNP in a sequence (See, Saiki et al. (1986) Nature 324(6093):163-166 and U.S. Pat. No. 5,821,062; 7,052,845 or 7,250,258).

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. No. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. No. 5,576,180; 6,106,777 or 7,179,600) A recent modification on this process, known as Linear-After-The-Exponential-PCR (LATE-PCR), uses a limiting primer with a higher melting temperature (Tₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257)

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

Inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. No. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. No. 6,811,982; 6,835,541 or 7,125,673). DNA is first treated with sodium bisulfate, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. No. 5,882,856; 6,531,282 or 7,118,867). By targeting multiple genes at once, additional information may be gained from a single test run that otherwise would require several times the reagents and more time to perform. Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. No. 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. No. 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. No. 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TAIL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₘ of the primers used, while at the later cycles, it is a few degrees (3-5° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the invention, probes are labeled with two fluorescent dye molecules to form so-called “molecular beacons” (Tyagi, S, and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene of interest. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the genetic polymorphisms or gene expression levels identified herein. The prognostic panel comprises probes or primers that can be used to amplify and/or for determining the molecular structure of the polymorphisms or the gene expression levels identified herein. The probes or primers can be attached or supported by a solid phase support such as, but not limited to a gene chip or microarray. The probes or primers can be detectably labeled. This aspect of the invention is a means to identify the genotype of a patient sample for the genes of interest identified above.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

Nucleic Acids

In one aspect, the nucleic acid sequences of the gene of interest, or portions thereof, can be the basis for probes or primers, e.g., in methods for determining expression level of the gene of interest or the allelic variant of a polymorphic region of a gene of interest identified in the experimental section below. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to the gene of interest. Thus, such primers can be specific for the gene of interest sequence, so long as they have a nucleotide sequence which is capable of hybridizing to the gene of interest. Examples of primers and probes useful in the herein described invention are shown in Tables 1 and 2. The VEGF allele with polymorphism G-634C is identified and described in Sfar (2006) 35(1-2):21-28. Furthermore, the VEGF G-634C polymorphism is also known in the art as VEGF G+405C as described in Buraczynska et al. (2006) Nephrol. Dial Transplant doi:10.1093/ndt/gfl641 and Shastry et al. (2006) Graefe's Archive for Clinical and Experimental Ophthalmology 245(5):741-743. The IL-1Ra VNTR polymorphisms are identified and described in Vijgen et al. (2002) Genes and Immunity 3:400-406.

TABLE 1
Probe and Primer Sequences for
Determining Gene Expression Levels

| | Forward | Reverse | Taqman |
|---|---|---|---|
| Gene | Primer(5′-3′) | Primer (5′-3′) | Probe(5′-3′) |
| β-actin | GAGCGCGGC | TCCTTAATGTC | ACCACCACG |
| | TACAGCTT | ACGCACGATTT | GCCGAGCGG |
| TS | GCCTCGGTG | CCCGTGATG | TCGCCAGCTAC |
| | TGCCTTTCA | TGCGCAAT | GCCCTGCTCA |
| HIF1α | CGCTGGAGACA | TCCTCAAGTT | TTTGGCAGCAAC |
| | CAATCATATC | GCTGGTCATC | GACACAGAAACT |
| LDHA | TGATGGATCT | CAGCTTGGAGT | CCTTAGAACACCAA |
| | CCAACATGG | TTGCAGTTAC | AGATTGTCTCTGGC |
| Glut-1 | CAGCAGCAAG | GAGCCAAGCA | TCGCCTCATG |
| | AAGCTGACG | CTGCTCCTC | CTGGCTGTG |
| VEGF | AGTGGTCCC | TCCATGAACTT | ATGGCAGAAGGAG |
| | AGGCTGCAC | CACCACTTCGT | GAGGGCAGAATCA |
| VEGFR1 | CGCATATGGTA | AGTCACACCTT | TGGTTCTGGCACC |
| | TCCCTCAACCT | GCTTCGGAATG | CCTGTAACCATAA |
| VEGFR2 | CCTGTGGCT | CTGAGCCTGG | CACTAGGCAAACC |
| | CTGCGTGGA | GCAGATCAAG | CACAGAGGCGGC |

TABLE 2
Primer Sequences, Annealing
Temperatures and Restriction
Enzymes for Determining Polymorphisms

| | Forward- | Reverse- | | |
|---|---|---|---|---|
| | Primer | Prime | | Anneal- |
| Gene | (5′-3′) | (5′-3′) | Enzyme | ing |
| PAR-1 | ACTGTCGACG | ATTCGCGAAG | N/A | 60 |
| I-506D | TCTCCACATC | CTGTCAGTG | | |
| ES | CACGGTTTCT | CTCTCAGAGC | Mse I | 60 |
| G+4349A | CTTCCAGGAC | TGCTCACACG | | |
| IL-8 | TTGTTCTAACA | GGCAAACCTG | Mfe I | 60 |
| T-251A | CCTGCCACTCT | AGTCATCACA | | |
| IL-1β | GGCCTGCCCT | ATGGACCAGA | Taq a1 | 60 |
| C+3954T | TCTGATTTTA | CATCACCAAG | | |
| IL-1Ra | CTCAGCAAC | TCCTGGTCT | N/A | 60 |
| VNTR | ACTCCTAT | GCAGGTAA | | |
| ICAM-1 | CCATCGGGG | ACAGAGCACA | BstU1 | 60 |
| K496E | AATCAGTG | TTCACGGTC | | |
| GRP78 | CTGGATCCCA | AGGTGGTCCA | N/A | 60 |
| (rs12009) | ACACCAAAT | CGGTAGTGAG | | |
| NFkB | CTTCAGTATCT | CAAGTAAGACT | N/A | 60 |
| CA repeat | AAGAGTATCCT | CTACGGAGTC | | |
| K-RAS | TGACTGAATA | TCGTCCACAAA | N/A | 62 |
| codon 12 | TAAACTTGTG | ATGATTCTGA | | |
| | GTAGTTG | | | |
| K-RAS | TGACTGAATAT | TCGTCCACAAA | N/A | 62 |
| codon 13 | AAACTTGTGGT | ATGATTCTGA | | |
| | AGTTG | | | |
| VEGF | ACT TCC CCA | GTC ACT CAC | Se- | 60 |
| G-634C | AAT CAC TGT | TTT GCC CCT | quencing | |
| | GG | GT | | |

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Methods of Treatment

The invention further provides methods of treating patients having solid malignant tissue mass or tumor from a gastrointestinal cancer, e.g., rectal cancer, colorectal cancer, colon cancer, gastric cancer, and esophageal cancer. In another aspect, the invention provides methods for treating patients having stage II colon cancer, stage II rectal cancer or stage III rectal cancer. In a further aspect, the above cancers are non-metastatic or metastatic. In yet a further aspect, the stage II colon cancer has not spread to the lymphatic system. Without being bound by theory, Applicants intend that the methods are also useful to treat patients identified to likely to respond to the combination therapy when the patient is suffering from lung cancer, ovarian cancer, head and neck cancer or hepatocarcinoma as these cancers have been successfully treated with an effective amount of a pyrimidine based antimetabolite chemotherapy drug and a platinum based chemotherapy drug such as 5-FU and/or oxaliplatin and equivalents of each thereof alone or in combination with other inert carriers of no therapeutic significance to the combination.

In one embodiment, the patients of the above methods have not received previous chemotherapy treatment, wherein the administration of an effective amount of 5-FU based chemotherapy, 5-FU based adjuvant chemotherapy, FOLFOX/BV, XELOX/BV or a FOLFOX chemotherapy regimen and in some aspects in combination with PTK/ZK, or equivalents of each thereof is the first line therapy. In another embodiment, the patients of the above methods have previously received chemotherapy treatment for the patients. In some aspects the previous treatment comprised of a 5-fluorouracil and irinotecan based chemotherapy. In this aspect the administration of a FOLFOX chemotherapy regimen in combination with PTK/ZK or equivalents of each thereof is the second line therapy for the patients. In another aspect, the FOLFOX chemotherapy regimen comprises, for example, the combination of chemotherapies known in the art as FOLFOX4, which for the treatment of colon cancer includes, administration of oxaliplatin 85 mg/m² IV or 2 hours on day 1, leucovorin 200 mg/m² IV over 2 hours on days 1 and 2, followed on days 1 and 2 by 5-FU 300 mg/m² IV bolus, then 600 mg/m² IV over 22 hours continuous infusion, with repetition every 2 weeks.

In one embodiment, the method comprises (a) determining the presence of a polymorphism in the gene of interest or gene expression level of the gene of interest as identified herein; and (b) administering to the patient an effective amount of a compound or therapy (e.g., chemotherapy with 5-FU based chemotherapy, 5-FU based adjuvant chemotherapy, FOLFOX/BV, XELOX/BV or a FOLFOX chemotherapy regimen and in some aspects in combination with PTK/ZK, or equivalents of each thereof). This therapy can be combined with other suitable therapies or treatments as described herein.

The chemotherapy comprises, or alternatively consists essentially of, or yet further consists of administration of a pyrimidine based antimetabolite chemotherapy drug and a platinum based chemotherapy drug, e.g., 5-fluorouracil and oxaliplatin or FOLFOX or equivalents thereof, in an amount effective to treat the cancer and by any suitable means and with any suitable formulation as a composition and therefore includes a carrier such as a pharmaceutically acceptable carrier.

In another aspect, the chemotherapy comprises, or alternatively consists essentially of, or yet further consists of administration of a pyrimidine based antimetabolite chemotherapy drug, a platinum based chemotherapy drug and a tyrosine kinase inhibitor, e.g., 5-fluorouracil, oxaliplatin and PTK/ZK or FOLFOX+PTK/ZK or equivalents thereof, in an amount effective to treat the cancer and by any suitable means and with any suitable formulation as a composition and therefore includes a carrier such as a pharmaceutically acceptable carrier.

In another aspect, the chemotherapy or adjuvant chemotherapy comprises, or alternatively consists essentially of, or yet further consists of administration of a pyrimidine based antimetabolite chemotherapy drug based therapy, including, but not limited to FOLFOX (5-FU, leucovorin and oxaliplatin); FOLFIRI (5-FU, leucovorin and irinotecan) or 5-FU and leucovorin alone in an amount effective to treat the cancer and by any suitable means and with any suitable formulation as a composition and therefore includes a carrier such as a pharmaceutically acceptable carrier.

In yet another aspect, the chemotherapy comprises, or alternatively consists essentially of, or yet further consists of administration of a pyrimidine based antimetabolite, such as 5-FU, or a prodrug thereof, such as Capecitabine (Xeloda®), a platinum based chemotherapy drug, such as oxaliplatin and a VEGF antibody, such as Bevacizumab and in some aspects in combination with an efficacy enhancing agent, such as leucovorin (a.k.a—FOLFOX/BV or XELOX/BV) in an amount effective to treat the cancer and by any suitable means and with any suitable formulation as a composition and therefore includes a carrier such as a pharmaceutically acceptable carrier.

Accordingly, a formulation comprising the necessary chemotherapy or biological equivalent thereof is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

The chemotherapeutic agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON′S PHARM. SCI., 15th Ed. (Mack Publ. Co., Easton (1975) and Williams & Williams, (1995), and in the “PHYSICIAN′S DESK REFERENCE”, 52^{nd} ed., Medical Economics, Montvale, N.J. (1998).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, fluorouracil/oxaliplatin, methotrexate-leucovorin, and the like.

Combinations of chemotherapies and molecular targeted therapies, biologic therapies, and radiation therapies are also well known to the art; including therapies such as trastuzumab plus paclitaxel, alone or in further combination with platinum compounds such as oxaliplatin, for certain breast cancers, and many other such regimens for other cancers; and the “Dublin regimen” 5-fluorouracil IV over 16 hours on days 1-5 and 75 mg/m² cisplatin IV or oxaliplatin over 8 hours on day 7, with repetition at 6 weeks, in combination with 40 Gy radiotherapy in 15 fractions over the first 3 weeks) and the “Michigan regimen” (fluorouracil plus cisplatin or oxaliplatin plus vinblastine plus radiotherapy), both for esophageal cancer, and many other such regimens for other cancers, including colorectal cancer.

In another aspect of the invention, the method for treating a patient comprises, or alternatively consists essentially of, or yet further consists of surgical resection of a metastatic or non-metastatic solid malignant tumor and, in some aspects, in combination with radiation. Methods for treating said tumors derived from a gastrointestinal cancer, e.g., rectal cancer, colorectal cancer, colon cancer, gastric cancer, esophageal cancer, stage II colon cancer, stage II rectal cancer or stage III rectal cancer by surgical resection and/or radiation are known to one skilled in the art. Guidelines describing methods for treatment by surgical resection and/or radiation can be found at the National Comprehensive Cancer Network's web site, nccn.org, last accessed on May 27, 2008.

The invention provides an article of manufacture, comprising packaging material and at least one vial comprising a solution of the chemotherapy as described herein and/or or at least one antibody or its biological equivalent with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising the chemotherapy and/or at least one lyophilized antibody or its biological equivalent and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

When an antibody is administered, the antibody or equivalent thereof is prepared to a concentration includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Chemotherapeutic formulations of the present invention can be prepared by a process which comprises mixing at least one antibody or biological equivalent and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject® NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a chemotherapeutic agent of the invention, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a medicament comprising an effective amount of a chemotherapeutic as described herein for treatment of a human cancer patient having high or low gene expression or the polymorphism of the gene of interest as identified in the experimental examples.

Kits

As set forth herein, the invention provides diagnostic methods for determining the polymorphic region or expression level of the gene of interest. In some embodiments, the methods use probes or primers comprising nucleotide sequences which are complementary to the gene of interest. Accordingly, the invention provides kits for performing these methods as well as instructions for carrying out the methods of this invention such as collecting tissue and/or performing the screen, and/or analyzing the results, and/or administration of an effective amount of a 5-FU based chemotherapy, 5-FU based adjuvant chemotherapy, FOLFOX/BV, XELOX/BV or a FOLFOX chemotherapy regimen and in some aspects in combination with PTK/ZK, or equivalents of each thereof. These can be used alone or in combination with other suitable chemotherapy or biological therapy.

In an embodiment, the invention provides a kit for determining whether a subject is likely responsive to cancer treatment or alternatively one of various treatment options. The kits contain one of more of the compositions described above and instructions for use. As an example only, the invention also provides kits for determining response to cancer treatment containing a first and a second oligonucleotide specific for the polymorphic region of the gene. Oligonucleotides “specific for” the gene of interest bind either to the gene of interest or bind adjacent to the gene of interest. For oligonucleotides that are to be used as primers for amplification, primers are adjacent if they are sufficiently close to be used to produce a polynucleotide comprising the gene of interest. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

The test samples used in the diagnostic kits include cells, protein or membrane extracts of cells, or biological fluids such as sputum, blood, serum, plasma, or urine. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

Other Uses for the Nucleic Acids of the Invention

The identification of the polymorphic region or the expression level of the gene of interest can also be useful for identifying an individual among other individuals from the same species. For example, DNA sequences can be used as a fingerprint for detection of different individuals within the same species. Thompson, J. S. and Thompson, eds., (1991) GENETICS IN MEDICINE, W B Saunders Co., Philadelphia, Pa. This is useful, e.g., in forensic studies.

The invention now being generally described, it will be more readily understood by reference to the following example which is included merely for purposes of illustration of certain aspects and embodiments of the present invention, and are not intended to limit the invention.

### EXPERIMENTAL EXAMPLES

### Example 1

### Sex, Age and Ethnicity are Associated with Survival in Metastatic Colorectal Cancer

Background: At all ages, women are less likely to develop colorectal cancer (CRC) than men. Farquhar et al. (2005) Database Syst. Rev. CD004143. In fact their risk is comparable to men aged between 4-8 year younger. Brenner et al. (2007) Br J Cancer 96(5):828-31. Gender differences have also been associated with tumor biology, therapeutic response, and disease prognosis. Dietary and genetic differences may explain these inequalities, but evidence is accumulating for a hormonal etiology.

The Women's Health Initiative confirmed that post-menopausal hormone use is associated with a 40% decrease in colorectal cancer. Chlebowski et al. (2004) N Engl J Med 350(10):991-1004. Although, the role of estrogen in CRC tumorigenesis is unclear, investigators have found that estrogen receptor β is selectively lost in malignant colonic tissue. Foley et al. (2000) Cancer Res 60(2):245-248.

Age and ethnicity have been shown to impact the survival rates of men and women with metastatic colorectal cancer (MCRC). Yet gender is neither prognostic nor predictive for overall survival (OS). We investigated the interactions between sex, age, and ethnicity on overall survival in patients with MCRC.

Methods: 56,598 patients with mCRC from 1988-2003 were screened, using the Surveillance, Epidemiology, and End Results (SEER) registry. All patients received 5-FU based chemotherapy. Age at diagnosis, sex, ethnicity and overall survival were evaluated using Cox proportional hazards model. The models were adjusted for marital status, tumor site, and treatment with radiation and/or surgery. Models were stratified by SEER registry site and year of diagnosis.

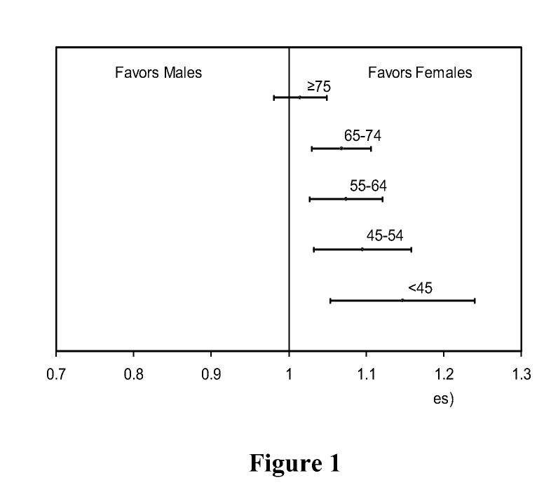

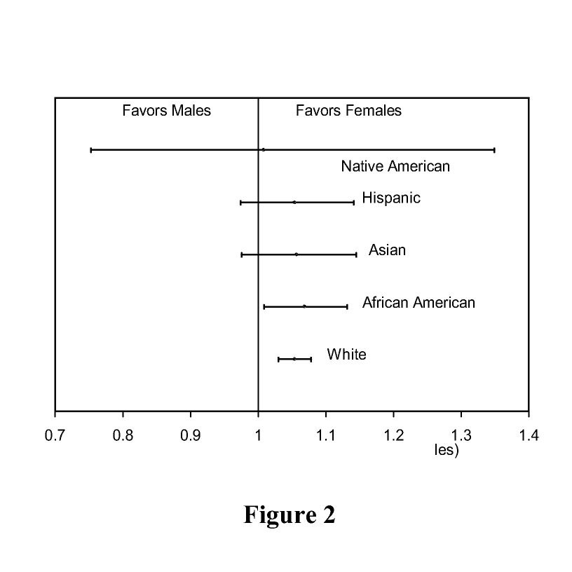

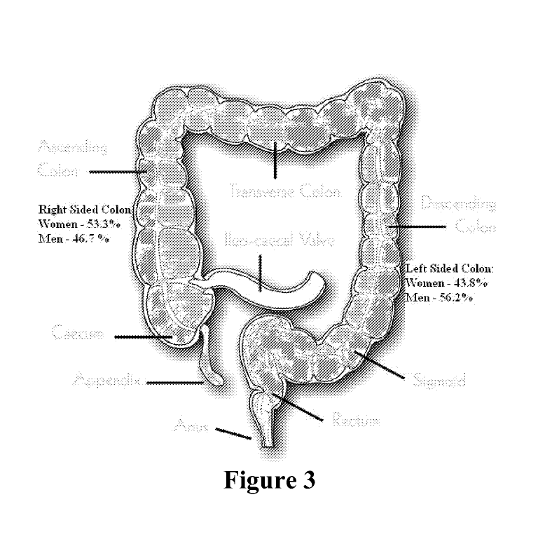

Results: Independent of age, there were no survival differences between men and women with mCRC. However, when age was added to the model, sex became significantly associated with survival across all ethnicities (p<0.0001). Younger women (18-44 years old) with mCRC lived longer than younger men (17 months vs. 14, p<0.0001). In contrast, older women (75 and older) had significantly worse overall survival than older men (p<0.0001, Table 3). As women age their risk becomes equivalent to men (FIG. 1). This association was independent of ethnicity (FIG. 2). Women were more likely to have right sided colon lesions and men more likely to have left sided colon lesions (P<0.0001, FIG. 3).

TABLE 3
Overall survival of patients with mCRC by age and sex

| | | | | | Female | | | | |
|---|---|---|---|---|---|---|---|---|---|
| | Male | | | | | OS | | | |
| Age, | | OS | | | | me- | | | P |
| years | N | median | 95% | CI | N | dian | 95% | CI | value* |
| 18-44 | 1490 | 14 | 13 | 15 | 1526 | 17 | 16 | 17 | <.0001 |
| 45-54 | 3398 | 13 | 13 | 14 | 2830 | 15 | 14 | 15 | 0.0014 |
| 55-64 | 6351 | 12 | 12 | 12 | 4520 | 12 | 12 | 12 | 0.14 |
| 65-74 | 8819 | 9 | 8 | 9 | 7113 | 9 | 8 | 9 | 0.15 |
| ≧75 | 9209 | 5 | 4 | 5 | 11341 | 4 | 4 | 4 | <.0001 |

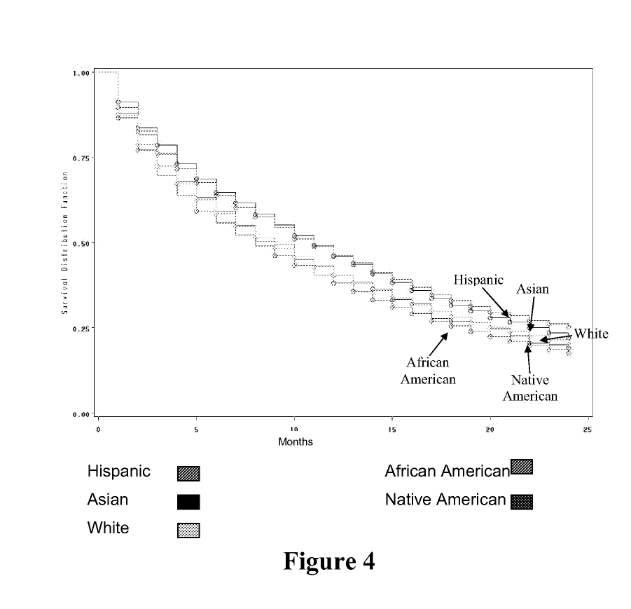

In multivariate analysis, age and ethnicity were significantly associated with survival. Across all age deciles, Hispanics had the longest overall survival, followed by Whites, Asians, African Americans, and Native Americans, respectively (p<0.0001, FIG. 4). Across all ethnicities younger patients had a better prognosis except for Native Americans; Patients from 18-44 years had the worst prognosis and an overall survival of 8 months.

Conclusion: These results show that sex, age and ethnicity have a significant impact on overall survival in mCRC patients. As one of the largest data sets analyzed, these results establish that younger women of all ethnicities survive longer than younger men. Thus, hormonal status appears to play an important role not only in the development and pathogenesis of colorectal cancer, but is of prognostic significance. This also lends support to the importance of sex-specific differences in EGFR and MTHFR polymorphisms, as prognostic markers in CRC.

### Example 2

### Age and Ethnicity Predict Overall Survival in Patients with Metastatic Gastric Cancer

Background: Patients diagnosed with metastatic gastric cancer have dismal outcome. There is a lack of established regimens to improve their survival. The prognostic role of gender, age and ethnicity on survival for patients with metastatic gastric cancer has not been determined in the U.S. It has been shown that Asians treated in Asia have overall a significantly better outcome than Caucasians treated in Western world. The etiology of gastric cancer may differ among the different ethnic groups. These different etiologies include environmental factors, H. pylori infection and dietary factors, which may lead to different genetic profiles of gastric cancer associated with different clinical outcome.

Methods: Extracting data from the US National Cancer Institute's Surveillance, Epidemiology, and End Results (SEER) registries, overall survival for patients with metastatic gastric cancer by gender, age and ethnicity was analyzed using multivariate Cox proportional hazards models. 15,228 patients (>18 years) were identified from the years 1988-2003. The ages of the males and females were categorized with <45, 45-54, 55-64, 65-74, and >75 years. Patients who were Native Americans, African Americans, Asians, Caucasians, and Hispanics were included. The models were adjusted for potential confounders including marital status at diagnosis, tumor site, treatment with radiation and/or surgery, and histology, and stratified by SEER registry sites and years of diagnosis.

Results: Overall survival was decreased with age (p<0.001, Cox model). The median overall survival was 6 months in patients of ≦44 years compared to 2 months in patients who were >75 years. The difference of overall survival by ethnicity was significantly varied by sex (P for interaction=0.01). Among males, Asian patients had longer overall survival versus all other ethnicities, and African American patients had shorter overall survival compared to Caucasian patients (p=0.023). No significant difference in overall survival across ethnicity was found among females.

Conclusions: This is the largest study of metastatic gastric cancer from SEER registries to show that age was a significant prognostic factor for overall survival in patients with metastatic gastric cancer. The influence of ethnicity on overall survival was dependent on sex.

### Example 3

### Polymorphisms in PAR-1, ES and IL-8 Predict Tumor Recurrence in Patients with Surgically Resected Gastric Cancer

Background: Tumor recurrence continues to be a significant problem in the management of patients with surgically resected gastric cancer. Thrombin-receptor 1 (PAR1) has been described to counter-regulate the release of endostatin (ES) and VEGF from human platelets. PAR-1 could therefore play a crucial role in the regulation of tumor angiogenesis and in turn may regulate the process of tumor invasion and metastasis. Further, interleukin-8 (IL-8) has been reported to play a major role in VEGF-independent tumor angiogenesis. Fourteen functionally significant gene polymorphisms within 8 genes involved in the tumor angiogenesis pathway were tested to determine which polymorphisms predict tumor recurrence in patients with surgically resected gastric cancer.

Methods: Between 1992 and 2007 blood specimens from 105 patients (41 females and 64 males; median age=57 yrs; range=26-85 yrs) were obtained at the University of Southern California medical facilities, Norris Comprehensive Cancer Center and USC-Los Angeles County Medical Center (Table 4). The median follow-up was 2.4 years (range=0.1-12.3). 47 of 105 patients (45%) developed tumor recurrence with a 5-year probability of 0.38±0.06. Genomic DNA was isolated from peripheral blood and genotypes were determined using PCR-RFLP.

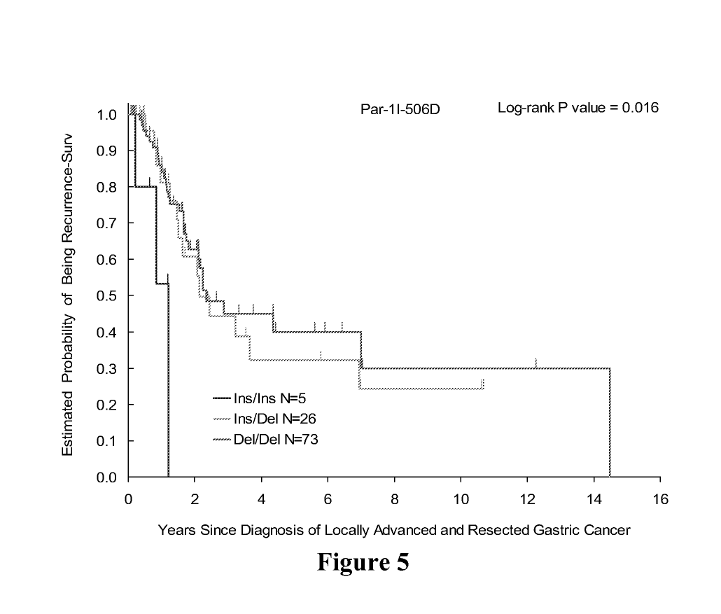

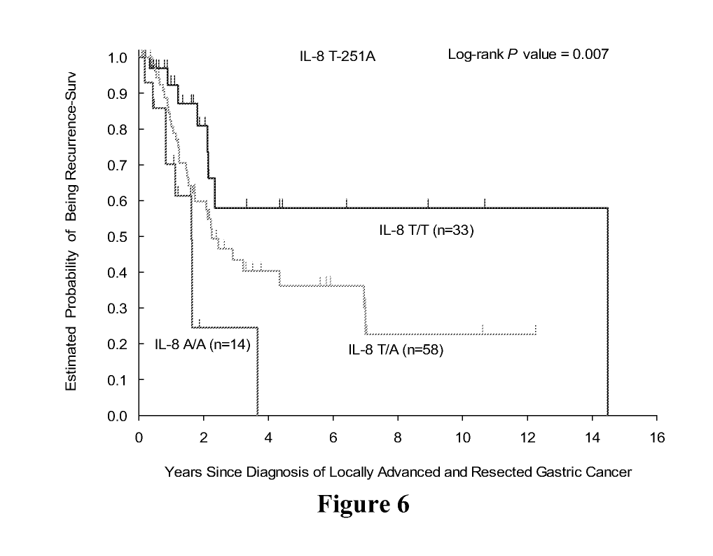

Results: High-expression variant genotypes (ins/ins) of the PAR-1 I-506D 13-bp insertion polymorphism (FIG. 5, median TTR: 1.2 yrs) and (A/A) of the IL-8 T-251A polymorphism (FIG. 6, median TTR: 1.6 yrs) as well as low-expression variants (A/A) of the ES G+4349A polymorphism (median TTR: 2.2 yrs) were associated with an increased likelihood of tumor recurrence, compared to other genotype combinations of (del/del or ins/del) for PAR-1 I-506D (median TTR: 2.3 yrs), (T/T or T/A) for IL-8 T-251A (median TTR: 2.9 yrs), and (G/G or G/A) for the ES G+4349A (median TTR: 2.9 yrs) polymorphisms. In multivariate analysis, polymorphisms in PAR-1 (adjusted p=0.04) and IL-8 (adjusted p=0.03) showed to be independent prognostic factors for TTR.

TABLE 4
Patient Population Characteristics, Time to Tumor Recurrence, and Relative Risk for
Surgically Resected Gastric Cancer Patients

| | | | Median time to recurrence | | | |
|---|---|---|---|---|---|---|
| | | | (TTR) | Relative risk | Probability ± SE* | |
| | N = 105 | % | yrs (95% CI) | (95% CI) | of 3-year recurrence | P value † |
| Age | | | | | | 0.87 |
| <50 | 27 | 26 | 2.2 (1.7, 14.5+) | 1 | 0.51 ± 0.13 | |
| 50-59 | 35 | 33 | 1.8 (1.5, 8.9+) | 1.03 (0.47, 2.27) | 0.53 ± 0.10 | |
| 60-69 | 27 | 26 | 2.3 (2.1, 12.3+) | 0.98 (0.43, 2.22) | 0.64 ± 0.13 | |
| ≧70 | 16 | 15 | 2.5 (1.0, 7.0+) | 1.37 (0.57, 3.31) | 0.57 ± 0.15 | |
| Sex | | | | | | 0.92 |
| Male | 64 | 61 | 2.2 (1.7, 4.4) | 1 | 0.59 ± 0.07 | |
| Female | 41 | 39 | 3.7 (1.7, 7.0+) | 0.97 (0.52, 1.82) | 0.43 ± 0.10 | |
| Race | | | | | | 0.021 |
| White | 36 | 34 | 1.7 (1.1, 2.2) | 1 | 0.73 ± 0.08 | |
| Black | 1 | 1 | 0.5 | — | 0 | |
| Asian | 24 | 24 | 7.0 (2.3, 14.5+) | 0.38 (0.18, 0.81) | 0.45 ± 0.13 | |
| Hispanic | 44 | 44 | 3.7 (2.1, 10.7+) | 0.47 (0.24, 0.92) | 0.42 ± 0.10 | |
| Stage | | | | | | 0.36 |
| I | 18 | 17 | 2.9 (2.2, 14.5+) | 1 | 0.50 ± 0.18 | |
| II | 24 | 23 | 3.7 (2.1, 10.7+) | 1.21 (0.42, 3.49) | 0.39 ± 0.12 | |
| III | 48 | 46 | 2.1 (1.5, 4.4) | 1.79 (0.70, 4.58) | 0.63 ± 0.09 | |
| IV | 15 | 14 | 1.6 (1.2, 3.8+) | 2.15 (0.69, 6.71) | 0.61 ± 0.15 | |
| GE junction | | | | | | 0.0214 |
| Yes | 21 | 20 | 2.1 (1.1, 2.5) | 1 | 0.85 ± 0.09 | |
| No | 84 | 80 | 3.7 (2.1, 14.5+) | 0.49 (0.26, 0.93) | 0.45 ± 0.07 | |

Conclusions: PAR-1 I-506D, ES G+4349A, and IL-8 T-251A polymorphisms identify gastric cancer patients who are at an increased risk to develop tumor recurrence. Thus targeting PAR-1, ES and IL-8 will be of clinical benefit in patients with surgically resected gastric cancer.

### Example 4

### Polymorphisms in IL-113 and IL-1Ra Predict Tumor Recurrence in Stage II Colon Cancer

Background: Identifying molecular markers for tumor recurrence is critical in successfully selecting patients with stage II colon cancer who are more likely to benefit from 5-FU based adjuvant chemotherapy. IL-1β and IL1-receptor antagonist (IL-1Ra) have been shown to play a critical role in the early initiation of tumor associated angiogenesis. In vitro and in vivo studies have shown that inhibition of the IL-1 receptor in IL-1β overexpressing tumors limits tumor angiogenesis and invasiveness. In this retrospective study, 7 functionally significant polymorphisms within 5 genes (IL-1β (a.k.a IL-1b), IL-1Ra, IL-8, CXCR1, CXCR2) in the chemokine family were tested for predicting the risk of tumor recurrence in stage II colon cancer patients treated with 5-FU based adjuvant chemotherapy.

Methods: Blood specimens from 107 patients (median age of 60 years; range: 22-86) were obtained at the University of Southern California medical facilities. All patients were diagnosed with high-risk, lymph node negative, stage II colon cancer and were uniformly treated with 5-FU based adjuvant chemotherapy. The median follow-up was 4.8 years (range: 0.3-16.8). 32 of 107 patients (29.9%) developed tumor recurrence with a 3-year probability of 0.23±0.04 (Table 5). Genomic DNA was extracted from peripheral blood and genotypes were determined using PCR-RFLP.

TABLE 5
Patient Population Characteristics, Time to Tumor Recurrence, and Relative Risk for Stage II
Colon Cancer Patients Treated with 5-FU based Adjuvant Chemotherapy

| | | Median time to | | Probability ± | |
|---|---|---|---|---|---|
| | | recurrence (TTR) | Relative risk | SE* of 3-year | |
| | n | yrs (95% CI) | (95% CI) | recurrence | P value † |
| Age, years | | | | | |
| Median, years (range) | 60 (22-86) | | | | 0.09 |
| ≦50 | 26 (24%) | 4.9 (3.5, 10.7+) | 1 | 0.26 ± 0.09 | |
| >50 | 84 (76%) | 16.8+ (9.4, 16.8+) | 0.53 (0.25, 1.14) | 0.18 ± 0.05 | |
| Sex | | | | | 0.72 |
| Male | 49 (45%) | 10.7 (4.9, 11.4+) | 1 | 0.15 ± 0.06 | |
| Female | 61 (55%) | 16.8+ (5.9, 16.8+) | 1.14 (0.55, 2.38) | 0.24 ± 0.06 | |
| Ethnicity | | | | | 1.00 |
| Caucasian | 52 (47%) | 10.7 (5.4, 16.8+) | 1 | 0.15 ± 0.05 | |
| African American | 7 (6%) | 10.3+ (0.8, 10.3+) | 1.06 (0.24, 4.60) | 0.29 ± 0.17 | |
| Asian | 8 (7%) | 10.6+ (4.9, 10.6+) | 0.92 (0.21, 4.02) | 0.13 ± 0.12 | |
| Hispanic | 43 (39%) | 8.5+ | 1.09 (0.47, 2.51) | 0.31 ± 0.09 | |
| T stage | | | | | 0.60 |
| T3 | 97 (88%) | 10.7 (9.4, 16.8+) | 1 | 0.20 ± 0.04 | |
| T4 | 13 (12%) | 5.4 (4.8, 10.9+) | 1.33 (0.46, 3.81) | 0.19 ± 0.13 | |
| N of resected lymph nodes | | | | | 0.95 |
| <12 | 41 (37%) | 10.7 (5.4, 11.4+) | 1 | 0.21 ± 0.07 | |
| ≧12 | 69 (63%) | 9.4 (5.9, 16.8+) | 0.98 (0.47, 2.02) | 0.19 ± 0.05 | |
| Adjuvant therapy | | | | | 0.01 |
| 5-FU | 73 (66%) | 9.4 (4.8, 16.8+) | 1 | 0.29 ± 0.06 | |
| 5-FU/Oxaliplatin | 25 (23%) | 10.7+ | 0.26 (0.06, 1.08) | 0 § | |
| 5-FU/CPT-11 | 12 (11%) | 13.3+ | 0 § | 0 § | |
| Tumor site | | | | | 0.22 |
| Left | 43 (40%) | 16.8+ (3.5, 16.8+) | 1 | 0.25 ± 0.07 | |
| Right ‡ | 61 (57%) | 10.7 (9.4, 13.3+) | 0.63 (0.30, 1.35) | 0.14 ± 0.05 | |
| Left + Right ‡ | 3 (3%) | | | | |
| Differentiation | | | | | 0.47 |
| Well ‡ | 8 (8%) | | | | |
| Moderate ‡ | 83 (80%) | 10.7 (9.4, 16.8+) | 1 | 0.22 ± 0.05 | |
| Moderate/Poor | 13 (12%) | 6.3+ (5.4, 6.3+) | 0.60 (0.14, 2.53) | 0 § | |

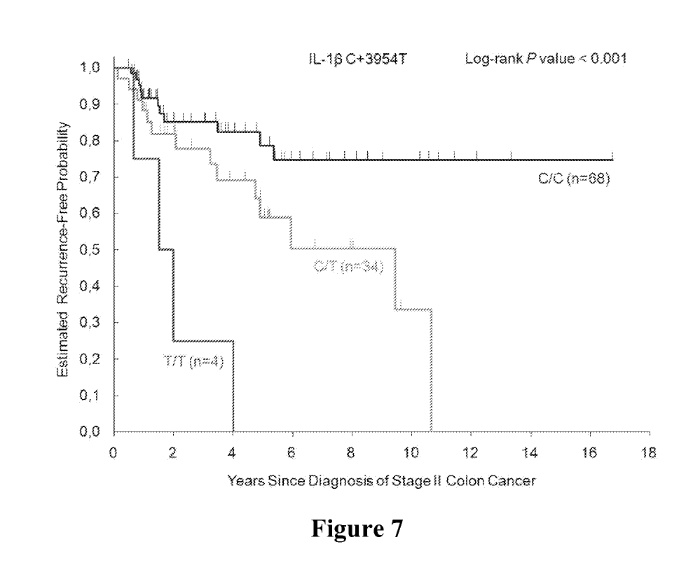

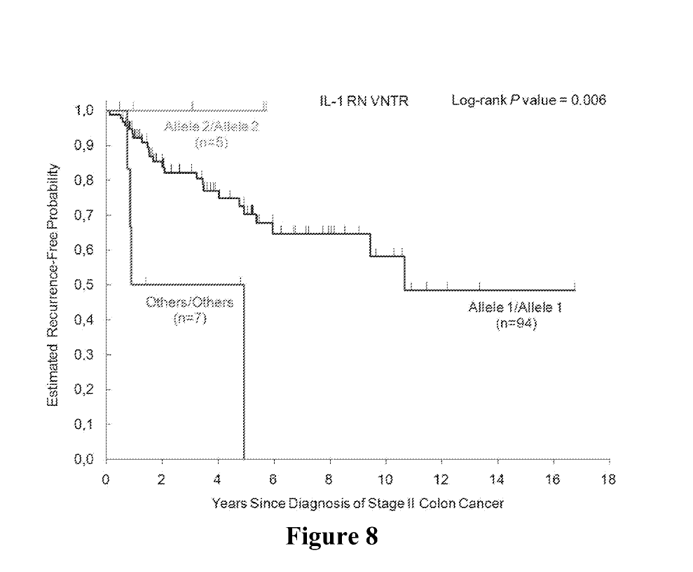

Results: Patients with genotypes (T/T) for the IL-1β C+3954T polymorphism (FIG. 7, median TTR: 1.5 years, p<0.001, log-rank test) or patients having at least one allele with >4 repeats for the IL-1Ra variable number of an 86-bp tandem repeat (VNTR) polymorphism, a.k.a Other/Other genotype, (FIG. 8, median TTR: 0.8 years, p=0.031, log-rank test) were associated with an increased likelihood for tumor recurrence, compared to genotype combinations of (C/C or C/T) for the IL-1β C+3954T polymorphism (median TTR: 10.7 years) or (4 repeats/4 repeats, a.k.a Allele 1/Allele 1 or 2 repeats/2 repeats, a.k.a. Allele 2/Allele 2) for the IL-1Ra VNTR polymorphism (median TTR: 10.7 years). In multivariate analysis, IL-1β C+3954T (adjusted p=0.030), IL-1 Ra VNTR (adjusted p=0.035), and VEGF G-634C (adjusted p=0.018) showed to be independent prognostic factors in stage II colon cancer. For example, the (T/T) genotype for IL-1β C+3954T, the (at least one allele with >4 repeats) genotype for IL-1 Ra VNTR or the (C/C or C/T) genotype for VEGF G-634C were predictive for shorter TTR.

Conclusion: IL-1β C+3954T, IL-1Ra VNTR and VEGF G-634C polymorphisms serve as independent molecular markers for TTR in stage II colon cancer. Therefore, the assessment of the individuals risk may be optimized on the basis of tumor-stage and specific genotypes, which will further enhance patient specific treatment not only by the identification of patients who are at high risk, but also by selecting more efficient treatment strategies. Furthermore, early initiation of chemokine mediated angiogenesis seems to play a critical role in colon cancer tumor relapse. Therefore, targeting IL-1 receptor can be of clinical benefit for stage II colon cancer patients.

### Example 5

### Ethnicity is Associated with Recurrence in Patients with Resected Gastric Cancer

Background: Previous studies suggest that there are differences in survival among Asian and Caucasian patients with gastric cancer. It remains unclear whether disparities of treatment, differences in staging, or individual tumor biology account for this effect. The present study examines differences among ethnicities in time to tumor recurrence (TTR) in patients with resected gastric cancer. Polymorphisms in thrombin-receptor 1 (PAR1), endostatin (ES), and interleukin 8 (IL-8) are important in angiogenesis and have been implicated in gastric cancer recurrence. The frequency of these polymorphisms was tested to provide a molecular basis for the differences in TTR for these different ethnic groups.

Methods: Between 1992 and 2007, 105 patients (41 females and 64 males with a median age of 57 yrs; range=26-85) with gastric adenocarcinoma underwent gastrectomy at the University of Southern California. There were 36 Caucasian, 1 African American, 24 Asian, and 44 Hispanic patients. The median follow-up was 2.4 years (range=0.1-12.3). 47 of 105 patients (45%) developed tumor recurrence with a 5-year probability of 0.38±0.06. Allelic frequencies of PAR1, ES, and IL-8 were determined using PCR-RFLP.

Results: Time to recurrence was significantly different by ethnic background (p=0.021, log-rank test). The median TTR for Asians was 7.0 yrs, for Hispanics was 3.7 yrs, and for Caucasians was 1.7 yrs. Gastroesophageal (GE) junction tumors had a shorter TTR compared to other tumor locations (2.1 yrs vs. 3.1 yrs, p=0.021). Allelic frequencies in genes for PAR1 and ES were statistically different between Asians, Hispanics, and Caucasians (PAR1 p=0.008; ES p=0.05), but there were no statistical differences for IL-8. Multivariate analyses including sex and age showed that ethnicity remained a significant TTR.

Conclusion: These results demonstrates significant differences in time to recurrence after gastrectomy favoring Asians and Hispanics. Genes in PAR1 and endostatin may provide a molecular basis for differences in TTR and may suggest different biologies of cancer between Caucasian, Hispanic, and Asian populations.

### Example 6

### Polymorphisms in ICAM-1, GRP-78 and NFkB Predicted Clinical Outcome in Patients with Metastatic Colorectal Cancer

Background: VEGF-targeted, anti-angiogenic therapy has significantly improved therapeutic success in metastatic colorectal cancer (mCRC) patients. However, no predictive or prognostic molecular markers have been identified in association with VEGF-targeted therapy. Polymorphisms of genes involved in angiogenesis, cell proliferation, and cell-cell or cell-matrix adhesion were evaluated as potential predictors of clinical outcome in patients with metastatic colorectal cancer (mCRC) who received Bevacizumab (BV) as part of their frontline FOLFOX or XELOX therapy. These genes included: VEGF, VEGF receptor 2, neuropilin 1, Interleukin 6 and 8, adrenomedullin, leptin, fibroblast growth factor receptor 4, tissue factor, matrix metalloproteinases 2,7,9, intracellular adhesion molecule-1 (ICAM-1), glucose regulated protein 78 (GRP78), epidermal growth factor receptor, and nuclear factor kappa b (NFkB).

Methods: A total of 59 patients with metastatic colorectal cancer treated who received first-line treatment with FOLFOX or XELOX in combination with bevacizumab at the University of Southern California are included in this study (Table 6). All patients gave informed consent. Patient information was collected through prospective database review and retrospective chart review. The end point of this study was to identify molecular predictors of clinical outcome including response rate and progression free survival (PFS). The PFS was determined by calculating the difference between the date of first treatment and the date of last follow-up appointment or date of progression of disease.

Peripheral blood samples were collected from each patient and genomic DNA was extracted from white blood cells using the QiaAmp kit (Qiagen, Valencia, Calif.). PCR-RFLP assays were performed on genomic DNA extracted from the blood of all 59 patients as previously described.

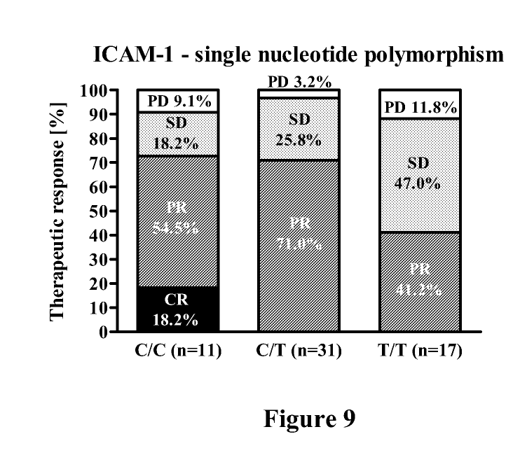

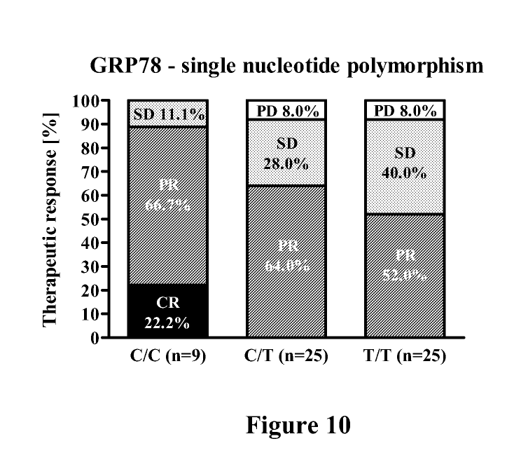

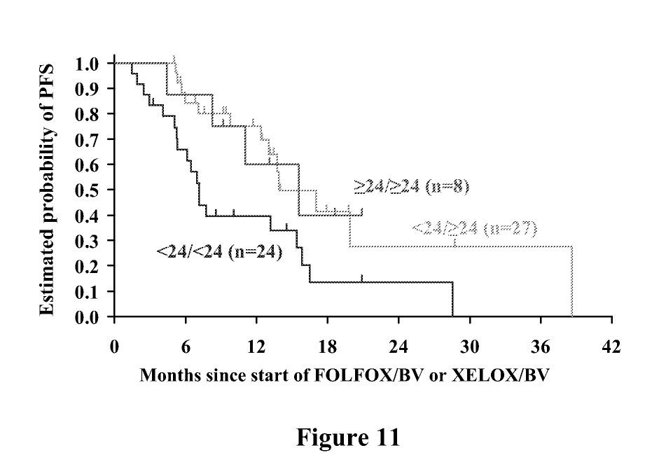

Results: There were 59 patients (36 males, 23 females), median age: 56 years (range: 29-81). 38 patients received XELOX/BV and 21 patients FOLFOX/BV (Table 6). Radiologic response: 2/59 (3%) complete response (CR), 35/59 (59%) partial response (PR), 18/59 (30.5%) stable disease and 4/59 (6%) progressive disease. At a median follow-up of 17.9 months (mo), 36/59 patients progressed with a median progression free survival (PFS) of 13.7 mo (Table 6). Single nucleotide polymorphisms (SNP)C/T in ICAM-1 (codon K469E, exon 6) and C/T in the 3′UTR region of GRP78 (rs12009) polymorphism were significantly associated with response (CR+PR). Patients homozygous for the T allele in ICAM-1 were found to have a lower probability of response (41%) compared to patients with the C/C (73%) or C/T (71%) genotypes (p=0.032) (FIG. 9). Patients with any C allele in the GRP78 gene (C/C; 89%) or (C/T; 64%) had a higher probability of response compared to patients homozygous for the T allele (T/T; 52%) (p=0.027) (FIG. 10). Significantly improved PFS was found in patients who had ≧24 CA repeats on at least one of the NFkB alleles located at 4q23-24 (15.5 mo [95% CI: 8.3-20.9+] for ≧24/≧24 genotype vs. 13.9 mo for <24/≧24 [95% CI: 13.0-38.6] vs. 7.2 mo [95% CI: 5.3-15.4] for <24/<24, p=0.023) (FIG. 11).

TABLE 6
Patient Population Characteristics Metastatic Colorectal
Cancer Patients

| Characteristics | Frequency | Percentage |
|---|---|---|
| Median age | 56 yrs | 29-81 yrs (range) |
| Gender | | |
| Female | 23 | 39 |
| Male | 36 | 61 |
| Treatment | | |
| FOLFOX/BV | 21 | 35.6 |
| XELOX/BV | 38 | 64.4 |
| Median follow-up | 17.9 months | 3.3-28.7 (range) |
| Progression free survival | 13.7 months | 95% CI: 8.3-16.5 |
| (PFS) Response | | |
| Complete Response (CR) | 2 | 3.4 |
| Partial Response (PR) | 35 | 59.3 |
| Stable Disease (SD) | 18 | 30.5 |
| Progressive Disease (PD) | 4 | 6.8 |

Receiving FOLFOX/BV or XELOX/BV Chemotherapy Regimens

Conclusions: The transcription factor family NFkB has been implicated in cell proliferation and angiogenesis. The main function of NFkB in tumors is to prevent apoptosis and to promote VEGF independent angiogenesis in response to chemotherapy and oxidative stress. Thus, NFkB dependent stress responses have been suggested to mediate resistance of tumors to anti-angiogenic therapy, chemotherapy and radiotherapy. De Martin et al. (2000) Arterioscler Thromb Vasc Biol 20:e83-e88.

ICAM-1 is pivotal for leukocyte-endothelial cell interaction and initiation of leukocyte-transmigration through the blood vessel wall. The sustained influence of angiogenic growth factor VEGF leads to its down-regulation which results in anergy of the tumor microvasculatur to inflammatory stimuli. Its microvascular anergy might protect the tumor from the host immune response. Anti-angiogenic therapy can reverse the microvascular anergy by normalizing the ICAM-1 expression levels and might therefore promote the host immune response to the tumor. Increased leukocyte infiltration in tumors has been associated with favorable clinical outcome in colorectal cancer patients. Griffioen (2008) Cancer Immunol Immunother DOI 10.1007/s00262-008-0524-3 and Baeten et al. (2006) Clin Gastroenterol Hepatol 4:1351-1357.

GRP78 (glucose-regulated protein 78) is a key survival factor in development and cancer. GRP78 expression is induced by cellular stress (glucose starvation, hypoxia) and inhibits pro-apoptotic effectors caspase-7, BIK, and prevents cytochrome c release. High expression levels of GRP78 have been previously associated with poor prognosis in colorectal cancer patients. Lee (2007) Cancer Res 67:3496-3499 and Xing et al. (2006) Clinica Chimica Acta 364:308-315.

These results demonstrate the predictive and prognostic value of ICAM-1, GRP78 and NFkB genomic polymorphisms in patients with mCRC treated with FOLFOX/BV or XELOX/BV.

### Example 7

### K-RAS Mutation Status Predicts Clinical Outcome in Patients with Metastatic Colorectal Cancer (mCRC)

Background: Activation of K-RAS mutations has been implicated in colorectal carcinogenesis. The most common mutations of K-RAS oncogene is in the exon 1 codons 12 and 13, which have been found in approximately 20-50% of colorectal cancers. Recent studies have shown K-RAS mutation status may predict response of mCRC patients to cetuximab, a chimeric anti-EGFR IgG1 monoclonal antibody. In this study, the K-RAS mutation was evaluated as being predictive for clinical outcome for mCRC patients receiving an anti-VEGF IgG1 monoclonal antibody, Bevacizumab (BV) as part of their first line therapy.

Methods: Tumor genomic DNA was extracted from 30 mCRC patients treated either with first line FOLFOX/BV or XELOX/BV at USC using laser capture microdissection technique. PCR and direct sequencing were used to determine the mutation status of K-RAS at codon 12 and 13.

Results: The cohort consisted of 21 males and 9 females with a median age of 56 years (range: 29-81). 20 patients received XELOX/BV as part of an on-going phase II study, 10 patients received FOLFOX/BV. Radiologic response was evaluable in 27/30 patients: 2/27 (7%) complete response (CR), 14/27 (52%) partial response (PR), 10/27 (37%) stable disease (SD) and 1/27 (4%) progressive disease. At a median follow-up of 19.4 months, 16/30 patients progressed with a median progression free survival (PFS) of 11.8 months. In 47% (14/30) of the patients, the codon 12 or 13 mutation was found. Patients with wild type K-RAS in codon 12 (GGT) and wild type K-RAS in codon 13 (GGC) have a better median progression free survival (median PFS=19.9 (13.7-26.9) months) compare to those with either codon 12 or codon 13 mutations (median PFS=8.3 (5.8-16.5) months) (p=0.061, log-rank test). No significant association between K-RAS mutation and response to Bevacizumab was found.

Conclusions: These results demonstrate the predictive value of K-RAS mutation in patients with mCRC treated with FOLFOX/BV or XELOX/BV.

### Example 8

### Gene Expression of TS in Tumor Tissue Predicts Overall Survival and Progression Free Survival for Patients with Stage II/III Rectal Cancer

Background: Clinical trials have shown postoperative chemoradiotherapy for adjuvant rectal cancer has improved overall survival and pelvic control. However, the efficacy of chemoradiation therapy may be significantly compromised as a result of individual variations in clinical response and host toxicity. At the G1 symposium 2008, preliminary data was reported that suggesting COX-2, IL-8 and TS-3′UTR gene polymorphisms may help to identify adjuvant rectal cancer patients who are more likely to experience longer survival. Gene-expression levels of genes involved in the critical pathways of cancer progression (i.e., drug metabolism (TS,TP,DPD,GSTP), tumor growth (COX-2, EGFR), angiogenesis (VEGF,IL-8), cell cycle regulation (CyclinD1,P53), and DNA repair (ERCC1,XPD)) were evaluated as predictors for clinical outcome in the same group of rectal cancer patients treated with 5-fluorouracil and pelvic radiation.

Methods: A total of 105 stage II/III rectal patients from a phase III trial (INT-0144, S9304) of three regimens of 5-fluorouracil and radiation were available for gene-expression assays. mRNA was extracted from laser-capture-microdissected tumor tissue. After cDNA was prepared by reverse transcription, quantitation of the candidate genes and an internal reference gene (β-actin) was performed using a fluorescence-based real-time detection method (TaqMan®).

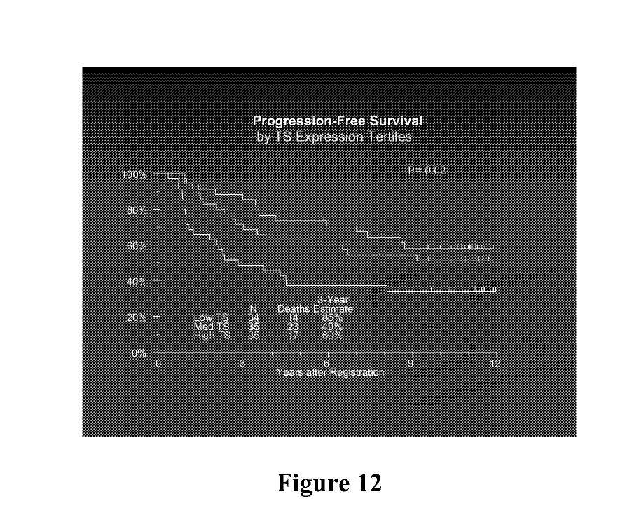

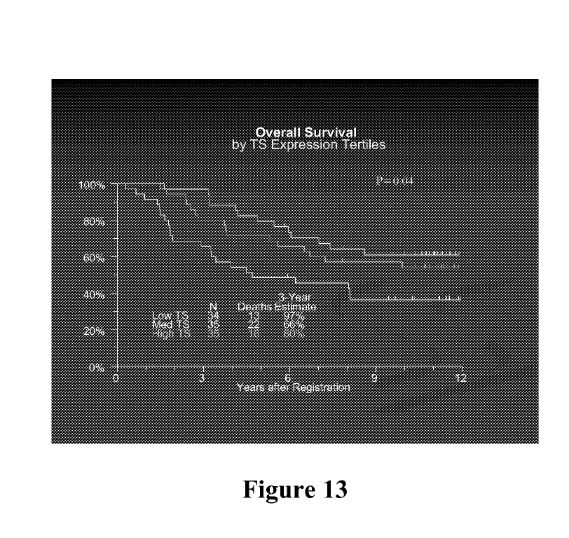

Results: In univariate analysis, TS gene expression levels was found to be significantly associated with overall survival (p=0.04, FIG. 12) and progression-free survival (p=0.02, FIG. 13). Patients with low TS expression levels showed better overall survival and progression-free survival compared to those with medium or high TS gene expression levels. All other genes tested did not show significant association with either overall survival or progression free survival.

Conclusions: These results demonstrate that gene expression levels of TS are predictive of PFS and OS for Stage II or III rectal cancer patients receiving 5-fluorouracil and radiation based therapy.

### Example 9

### Intratumoral Expression of Genes Involved in Angiogenesis and HIF1 Pathway Predict Outcome in Patients with Metastatic Colorectal Cancer

Background: Recent clinical trials (CONFIRM1 and CONFIRM2) have shown that patients with mCRC with high serum LDH benefited from PTK787/ZK 222584 (PTK/ZK), a VEGFR tyrosine kinase inhibitor (TKI). High intratumoral mRNA levels of genes involved with hypoxia (hypoxia inducible factor (HIF1α) and lactate dehydrogenase A (LDHA) and glycolysis (glucose transporter 1 (Glut-1) and genes involved in angiogenesis such as vascular endothelial growth factor A (VEGF) and its receptors (VEGFR1 and VEGF2) were tested as predictors for outcome in patients enrolled in CONFIRM1 and CONFIRM2 trials. The confirm trials are randomized, double-blind, placebo-controlled, phase III trials in patients with metastatic adenocarcinoma of the colon or rectum. Patients enrolled in the CONFIRM1 trial received first line treatment, whereas patients enrolled in the CONFIRM2 trial received second line therapy following progression from irinotecan-based therapy. PTK/ZK is an oral anti-angiogenic agent, which acts as a competitive inhibitor at the ATP-binding site of VEGF receptors 1-3, platelet-derived growth factor and c-kit. Wood et al. (2000) Cancer Res. 60:2178-2189.

Methods: 191 formalin fixed paraffin embedded (FFPE) tumor samples from patients enrolled in CONFIRM 1 and CONFIRM2 were analyzed. 85 patients from CONFIRM1 (42 patients treated with FOLFOX4, 43 patients with FOLFOX4/PTK) and 106 from CONFIRM2 (54 patients treated with FOLFOX4, 52 with FOLFOX4/PTK). FFPE tissues were dissected using laser-captured microdissection and analyzed for gene expression using the TaqMan® quantitative real-time RT-PCR method. Gene expression values (relative mRNA levels) were expressed as ratios between the gene of interest and the internal reference gene (β-actin). The maximally selected χ² method was used (1) to determine the optimal gene expression cut-off value and (2) to evaluate the association between the gene expression and clinical outcome. Gene expression levels were categorized as low or high expression using a threshold value for each gene. The following threshold values were used to determine high and low expression: LDHA-0.36 or 0.92; Glut1-1.5, 2.12, 3.25 or 3.28; VEGFR1-3.78 or 3.85, HIF1α-0.85, 1.18 or 1.21; VEGFR2-1.76, 1.78 or 2.98;

For example, a gene expression ratio for VEGFR1 below 3.78 or 3.85 was categorized as low expression, whereas a ratio above or equal to 3.78 or 3.85 was categorized as high expression. Associations between gene expression levels and outcome were evaluated by Mann Whitney U Test and are independently predictive.

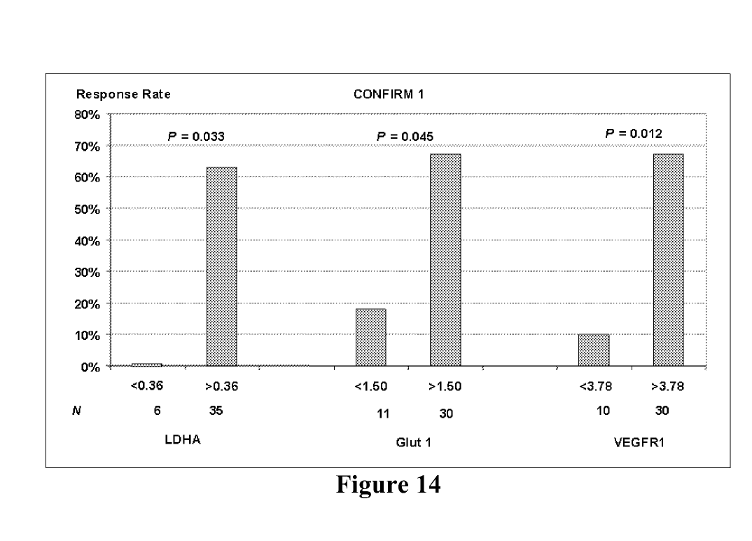

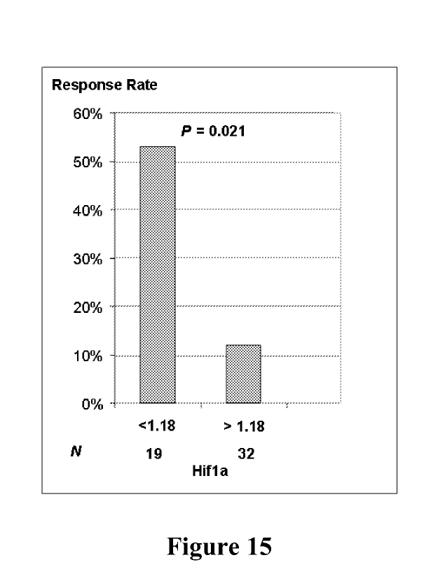

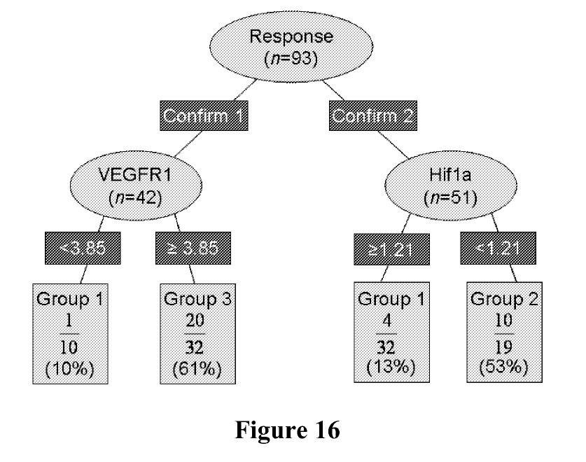

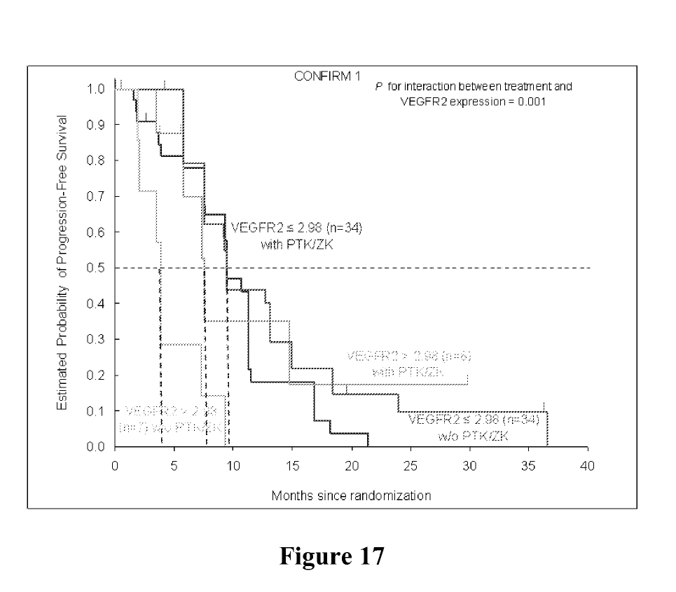

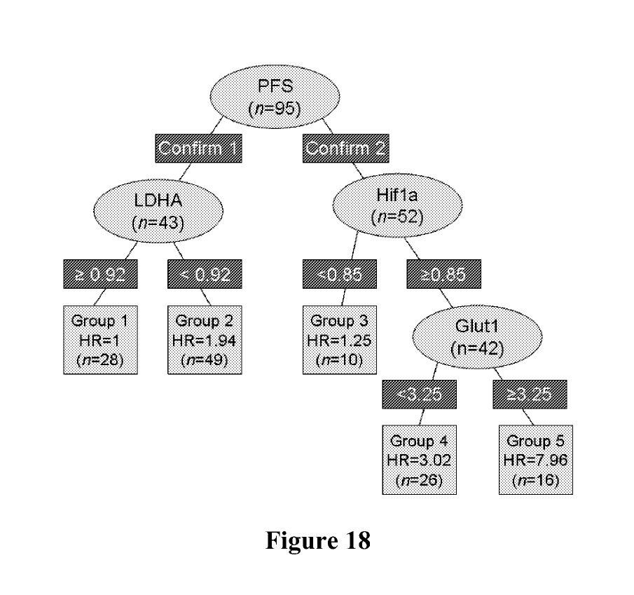

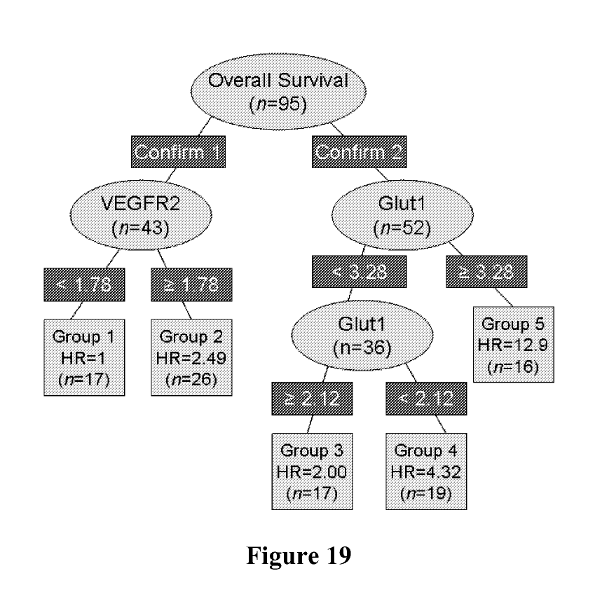

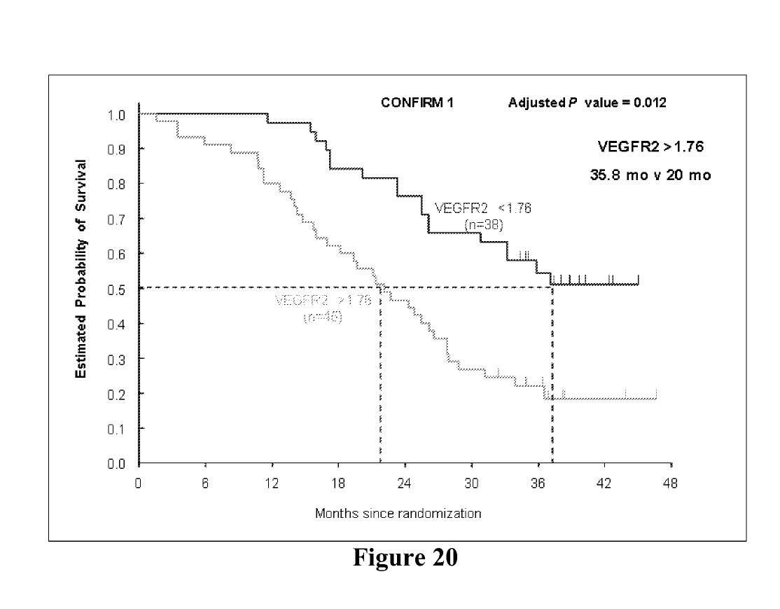

Results: High LDHA (p=0.033), high Glut1 (p=0.045) or high VEGFR1 (p=0.012) mRNA levels predicted improved tumor response in patients treated with FOLFOX4/PTK/ZK in CONFIRM1 but not in patients treated with FOLFOX4 (Table 7 and FIG. 14). Low HIF1α (p=0.021) gene expression predicted improved tumor response in patients treated with FOLFOX4/PTK in CONFIRM2 (Table 7 and FIG. 15). High HIF1α (p=0.021), high VEGFR2 (p=0.001) or high LDHA (p=0.075) mRNA levels were significantly associated with longer progression free survival (PFS) in patients treated with FOLFOX4/PTK in CONFIRM1 (Table 8, FIG. 16, FIG. 17 and FIG. 18). Low HIF1α (p=0.021), low LDHA (p=0.031) and combined high HIF1α and low Glut1 mRNA levels were significantly associated with longer PFS in patients receiving PTK in CONFIRM2 (Table 8 and FIG. 18). Low Glut1 (p=0.021) was associated with longer overall survival (OS) in patients treated with FOLFOX4/PTK in CONFIRM2, whereas low VEGFR2 (p=0.012) was associated with OS in patients treated with FOLFOX4/PTK in CONFIRM1 (Table 9, FIG. 19 and FIG. 20).

TABLE 7
Tumor Response Following Treatment with FOLFOX4/PTK/ZK

| | |
|---|---|
| CONFIRM 1 | CONFIRM 2 |

| Gene | p-value | Gene | p-value |
|---|---|---|---|
| >LDHA | 0.033 | <HIF-1α | 0.021 |
| >Glut1 | 0.045 | | |
| >VEGFR1 | 0.012 | | |

TABLE 8
Progression Free Survival Following Treatment with
FOLFOX4/PTK/ZK versus FOLFOX4

| | |
|---|---|
| CONFIRM 1 | CONFIRM 2 |

| Gene | Months | Gene | Months |
|---|---|---|---|
| >HIF-1α | 9.4 v 3.5 | <HIF-1α | 7.6 v 2.7 |
| PValue | 0.021 | PValue | 0.021 |
| >LDHA | 11.3 v 7.6 | <LDHA | 7.6 v 1.7 |
| PValue | 0.075 | PValue | 0.031 |
| >VEGFR2 | 8 v 4.1 | | |
| PValue | 0.001 | | |

TABLE 9
Overall Survival Following Treatment with FOLFOX4/PTK/ZK

| | |
|---|---|
| CONFIRM 1 | CONFIRM 2 |

| Gene | p-value | Gene | p-value |
|---|---|---|---|
| <VEGFR2 | 0.012 | <Glut1 | 0.021 |

Conclusions: These results show that mRNA levels of genes involved in angiogenesis and in the HIF1α pathway are associated with response and progression free survival to treatment including a VEGFR tyrosine kinase inhibitor. Furthermore, genes in the VEGFR2 pathway have prognostic value for overall survival. These are the first data suggesting genes associated with specific PTK effectiveness.

### Example 10

### Intratumoral mRNA Expression of Genes Involved in Angiogenesis and HIF1α Pathway Predict Outcome to VEGFR Tyrosine Kinase Inhibition in Patients Enrolled in CONFIRM-1 and CONFIRM-2

In an expansion of the studies conducted in Example 9, the following studies and analyses were performed.

Background

PTK/ZK is a novel oral angiogenesis inhibitor that is active against all known VEGF receptor (VEGFR) tyrosine kinases and platelet-derived growth factor receptor (PDGFR) tyrosine kinases and, therefore, offers a novel approach to inhibiting tumor growth by angiogenesis [Sitaras 1988; Buchdunger 1995; Carmeliet 1996]. Two randomized, double-blind, placebo-controlled, phase III studies (CONFIRM-1 and CONFIRM-2) were carried out in patients with metastatic adenocarcinoma of the colon or rectum, who were receiving first line (CONFIRM-1) or second line (CONFIRM-2) chemotherapy with folinic acid (leucovorin), 5 fluorouracil, oxaliplatin (FOLFOX4) and either PTK/ZK or placebo. Subgroup analysis of interim data from CONFIRM-1 and CONFIRM-2 demonstrated that individuals with elevated serum LDH levels (more than 1.5 times the upper limit of normal) derived a greater clinical benefit when PTK/ZK was added to a standard FOLFOX4 regimen, compared to FOLFOX4 plus placebo.

Methods

Tissue Samples

Formalin fixed paraffin embedded (FFPE) colorectal adenocarcinoma samples were analyzed from patients enrolled in the clinical trials CONFIRM-1 and CONFIRM-2.

Microdissection

A pathologist reviewed FFPE tumor blocks for quality and tumor content. Ten sections, each of 1 μm thickness, were obtained from the areas identified with the highest tumor concentration and mounted on uncoated glass slides. Three sections representative of the beginning, middle and end of the tumor were taken for histological diagnosis and stained with hematoxylin and eosin using the standard method. Before micro-dissection, sections were deparaffinized in xylene for 10 minutes, hydrated with 100%, 95% and 70% ethanol, and then washed in water for 30 seconds. Following this, the sections were stained with nuclear fast red (American Master Tech Scientific, Inc., Lodi, Calif.) for 20 seconds and rinsed in water for 30 seconds. Samples were dehydrated with 70% ethanol, 95% ethanol and 100% ethanol for 30 seconds each, followed by xylene for 10 minutes, and the slides were then air-dried. Laser capture micro-dissection (PALM Microlaser Technologies AG, Munich, Germany) was performed on all tumor samples to ensure that only tumor cells were dissected. The dissected areas of tissue were then transferred to a reaction tube containing 400 μL of RNA lysis buffer.

RNA Isolation and Complementary DNA Synthesis

Isolation of RNA from FFPE tumor samples was performed according to a proprietary procedure defined by Response Genetics, Inc. (Los Angeles, Calif.; U.S. Pat. No. 6,248,535). Complementary DNA was prepared as previously described (Lord et al., (2000) “Telomerase reverse transcriptase expression is increased early in the Barrett's metaplasia, dysplasia, adenocarcinoma sequence,” J Gastrointest Surg 4:135-142).

Reverse Transcription Polymerase Chain Reaction Quantification of Messenger RNA Expression

FFPE tumor samples were analyzed for gene expression using a quantitative real-time reverse transcription polymerase chain reaction (RT-PCR) method. Relative mRNA levels were expressed as ratios between the target gene and an internal reference gene (β-actin). Quantification of LDHA, Glut-1, HIF1α, VEGF, VEGFR1, VEGFR2 and β-actin was performed using a fluorescence-based real-time detection method (ABI PRISM 7900 Sequence detection System [TaqMan®] Perkin-Elmer [PE] Applied Biosystems, Foster City, Calif., USA). The PCR reaction mixture consisted of the following: 1200 nM of each primer, 200 nM of probe, 0.4 U of AmpliTaq Gold Polymerase, 200 nM each of dATP, dCTP, dGTP, dTTP, 3.5 mM MgCl₂, and 1×TaqMan Buffer A containing a reference dye, added to a final volume of 20 μl (all reagents from PE Applied Biosystems, Foster City, Calif., USA). Cycling conditions were 50° C. for 2 min, 95° C. for 10 min, followed by 46 cycles of 95° C. for 15 seconds then 60° C. for 1 min. The primer sequences and details of PCR conditions are included in Table 1.

Statistical Analysis

Tumor response was assessed per RECIST. Responders (complete or partial) were defined as patients in whom tumor burden had decreased by at least 50%. Non-responders were defined as patients with stable or progressive disease. Progression-free survival time was calculated as the period from the first day of randomization until the first observation of disease progression or death from any cause. If a patient had not progressed or died, progression-free survival was censored at the time of the last follow-up. The overall survival time was calculated as the time from the first day of randomization until death from any cause, or until the date of the last follow-up.

Gene expression values were stated as ratios between two absolute measurements: the gene of interest and the internal reference gene (β-actin). The associations between gene expression levels and response to therapy (responders vs. non-responders) were evaluated using a Mann-Whitney U test by trial and therapy. To assess the associations between the expression level of each gene and tumor response, progression-free survival time, or overall survival time, the expression level was categorized into a low and a high value at optimal cut-offs. The maximal χ² method of Miller and Siegmund (Miller and Siegmund (1982) “Maximally selected chi square statistics,” Biometrics 38:1011-1016) and Halpern (Halpern (1982) “Maximally selected chi square statistics for small samples,” Biometrics 38:1017-1023) was used to determine which gene expression value (optimal cut-offs) best segregated patients into poor- and good-prognosis subgroups, in terms of likelihood of response. The cut-off values chosen in analyzing response were applied for analysis of progression-free survival time or overall survival time. The analysis was conducted separately by trial and therapy. The Cox proportional hazards regression model was used to evaluate the independent effects of gene expression levels on progression-free survival and overall survival when adjusting baseline performance status and LDH level. Interactions between treatment and expression values were tested by comparing corresponding likelihood ratio statistics between the baseline and nested models that included the multiplicative product terms.

A classification and regression tree (CART) method, based on recursive partitioning (RP*), was used to explore gene expression variables for identifying homogenous subgroups for tumor response to therapy, progression-free survival time or overall survival time. (*The RP analysis is a nonparametric statistical method for modeling a response variable and multiple predictors. The RP analysis includes two essential processes: tree growing and tree pruning (Breiman et al., (1984) “Classification and Regression Trees,” Belmont, Calif.: Wadsworth).

No adjustment for multiple comparisons was performed. All reported p-values were two-sided. All analyses were performed using the SAS statistical package version 9.0 (SAS Institute Inc. Cary, N.C.), CART 5.0 (Salford Systems, San Diego, Calif.), and RPART function in the S-Plus library written by Therneau and Atkinson (Therneau and Atkinson (1997) “An Introduction to Recursive Partitioning Using the RPART Routines,” Mayo Clinic, Rochester, Minn. Technical Report 1997, number 61).

Results

Gene Expression Levels of LDHA, Glut-1, HIF1α, VEGF, VEGFR1 and VEGFR2

A total of 191 FFPE tumor samples from patients enrolled in CONFIRM-1 (n=85) and CONFIRM-2 (n=106) were analyzed. There were no statistically significant differences in baseline characteristics and gene expression levels by study and treatment—see Tables 10 and 11. Gene expression levels did not significantly vary by baseline serum LDH level (data not shown). Baseline characteristics and clinical outcome in patients from whom FFPE tissue was recovered were comparable to all patients in CONFIRM-1 and CONFIRM-2.

Gene Expression Levels by Treatment and Response

In CONFIRM-1, mRNA levels of LDHA (p=0.033), Glut-1 (p=0.045) and VEGFR1 (p=0.012) were predictive of a response to treatment in patients who received FOLFOX4 plus PTK/ZK, but not in patients who were treated with FOLFOX4 plus placebo; furthermore, there was a significant interaction to predict PTK/ZK activity for Glut-1 (p=0.036) and VEGFR1 (p=0.031)—see Table 12a. In CONFIRM-2, only HIF1α gene expression was predictive of a response to treatment in patients who received FOLFOX4 plus PTK/ZK (p=0.021)—see Table 12b. But there was no significant interaction between HIF1α and treatment on tumor response in CONFIRM-2 (data not shown).

Gene Expression Levels and Progression-Free Survival by Treatment

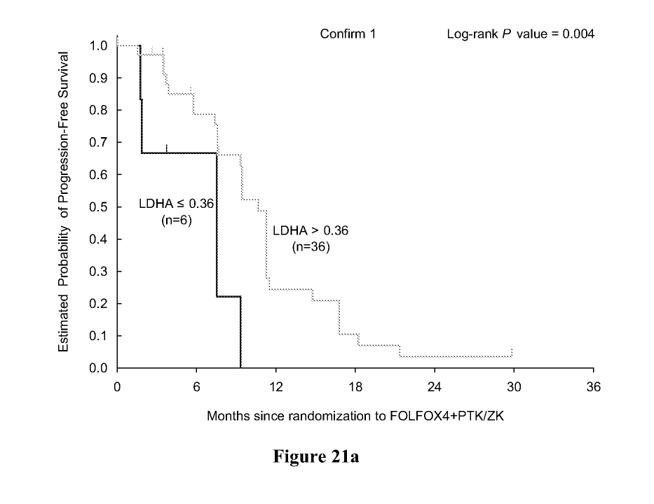

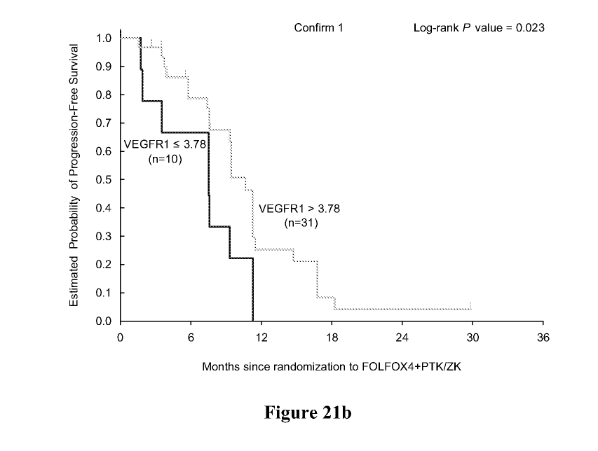

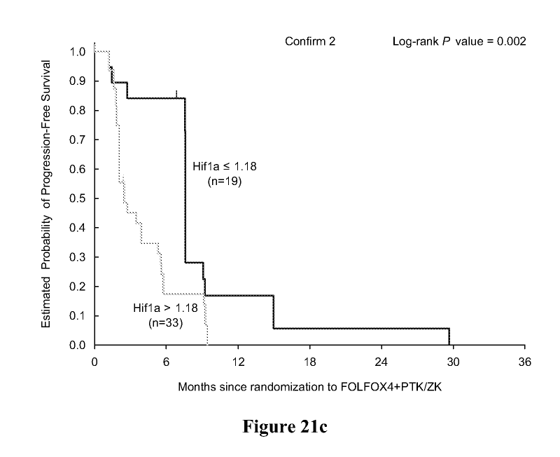

Progression-free survival was significantly associated with mRNA levels of LDHA (p=0.004) and VEGFR1 (p=0.023) in patients treated with FOLFOX4 plus PTK/ZK in CONFIRM-1, and with mRNA levels of HIF1α (p=0.002) in patients receiving FOLFOX4 plus PTK/ZK in CONFIRM-2—see Tables 13a and 13b, and FIGS. 21a-c, 22a and b. Additionally, there was a significant interaction for Glut-1 showing benefit for patients treated with PTK/ZK in CONFIRM-2 (p=0.038). All associations remained significant in the multivariate Cox model when adjusting baseline performance status and LDH level (p=0.013 for LDHA, p=0.036 for VEGFR1, and p<0.001 for HIF1α).

Gene Expression Levels and Overall Survival by Treatment

No significant association between gene expression levels and overall survival was demonstrated in either trial in patients treated with FOLFOX4 plus PTK/ZK—see Tables 14a and 14b. However, HIF1α and VEGFR2 expression were significantly associated with overall survival in patients receiving FOLFOX4 plus placebo in CONFIRM-1 (p=0.026 and p=0.003, respectively)—Table 14a. The associations between HIF1α and VEGFR2 expression and overall survival remained significant after adjusting baseline performance status and LDH level (p=0.008 for HIF1α and p=0.005 for VEGFR2). In addition, there was a significant interaction for VEGFR2 by treatment to predict survival in CONFIRM-1 (p=0.007).

Discussion

The data presented here suggest that intratumoral expression levels of genes involved in the HIF1α pathway are predictive and prognostic in patients with metastatic colorectal cancer treated using FOLFOX in combination with PTK/ZK. These data are consistent with the hypothesis that patients with increased serum LDH have significant up-regulation of VEGF and VEGFR1 gene expression in the tumor, and benefit from VEGFR TKI therapy. Furthermore, the data demonstrated that HIF1α pathway genes, such as LDHA, VEGF, VEGFR1, VEGFR2 and Glut-1 are significantly increased in patients with high serum LDH.

TABLE 10
Baseline characteristics by study and treatment

| | CONFIRM-1 patients | | | | CONFIRM-2 patients | | | |
|---|---|---|---|---|---|---|---|---|
| | who provided FFPE | | All CONFIRM-1 patients | | who provided FFPE | | All CONFIRM-2 patients | |
| | samples (N = 85) | | (whole trial) | | samples (N = 106) | | (whole trial) | |
| | FOLFOX4 + | | FOLFOX4 + | | FOLFOX4 + | | FOLFOX4 + | FOLFOX4 + |
| | PTK/ZK | FOLFOX4 + | PTK/ZK | FOLFOX4 + | PTK/ZK | FOLFOX4 + | PTK/ZK | Placebo |
| Characteristic | (n = 43) | Placebo (n = 42) | (n = 585) | Placebo (n = 583) | (n = 52) | Placebo (n = 54) | (n = 426) | (n = 429) |
| Sex: (%) | | | | | | | | |
| Male/Female | 31 (72)/12 (28) | 25 (60)/17 (40) | (62.9/37.1) | (60.4/39.6) | 34 (65)/18 (35) | 40 (74)/14 (26) | (62.0/38.0) | (62.5/37.5) |
| Age: years | | | | | | | | |
| Median (range) | 63 (28-79) | 62 (48-82) | 59.1 (24-89) | 59.6 (19-84) | 64 (45-83) | 62 (42-78) | 60.5 (21-85) | 59.2 (18-81) |
| PS^{a}: (%) | | | | | | | | |
| 0 | 23 (53) | 26 (62) | (55.9) | (55.6) | 25 (48) | 29 (54) | (54.0) | (52.4) |
| 1 or 2 | 20 (47) | 16 (38) | (43.9) | (43.9) | 27 (52) | 25 (46) | (46.0) | (47.5) |
| LDH^{b}: (%) | | | | | | | | |
| Low/high | 35 (81)/8 (19) | 31 (74)/11 (26) | (73.0/27.0) | (72.9/27.1) | 36 (69)/16 (31) | 43 (80)/11 (20) | (70.9/29.1) | (70.6/29.4) |

FFPE = formalin fixed paraffin embedded (tissue)
^{a}PS = World Health Organization performance status: 0 fully active and able to perform without restriction; 1 restricted physically strenuous activity; 2 capable of self-care but unable to work
^{b}LDH = lactate dehydrogenase: low ≦ 1.5 times upper limit of normal (ULN); high > 1.5 × ULN
There was no significant difference in baseline characteristics by study and treatment: all p values >0.05, not shown.

TABLE 11
Intratumoral gene expression levels by study and treatment

| | Patients from whom FFPE tissue samples were obtained | | | | | | | |
|---|---|---|---|---|---|---|---|---|
| | CONFIRM-1 | | | | CONFIRM-2 | | | |
| | (N = 85) | | | | (N = 106) | | | |
| | FOLFOX4 + | | FOLFOX4 + | | FOLFOX4 + | | FOLFOX4 + | |
| | PTK/ZK | | Placebo | | PTK/ZK | | Placebo | |
| Gene | N | Median (range) | N | Median (range) | N | Median (range) | N | Median (range) |
| LDHA | 42 | 1.11 (0.07-2.54) | 41 | 1.09 (0.14-3.63) | 52 | 0.91 (0.23-3.46) | 50 | 1.04 (0.20-2.66) |
| Glut-1 | 42 | 2.11 (0.26-23.86) | 41 | 2.49 (0.36-38.59) | 51 | 2.39 (0.45-13.37) | 50 | 3.45 (0.50-35.48) |
| HIF1α | 42 | 1.62 (0.59-3.68) | 41 | 1.62 (0.31-4.98) | 52 | 1.43 (0.60-6.45) | 50 | 1.43 (0.55-3.65) |
| VEGF | 42 | 6.21 (3.05-21.90) | 41 | 5.79 (1.29-15.90) | 52 | 5.71 (2.32-35.33) | 50 | 7.57 (2.54-65.06) |
| VEGFR1 | 41 | 6.28 (0.49-23.25) | 36 | 5.59 (0.56-28.06) | 48 | 5.33 (1.53-20.62) | 40 | 6.15 (2.54-17.83) |
| VEGFR2 | 42 | 1.91 (0.02-8.16) | 41 | 1.64 (0.62-11.63) | 50 | 2.11 (0.76-10.46) | 50 | 1.95 (0.49-14.48) |

FFPE = formalin fixed paraffin embedded;
LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
There was no significant difference in gene expression levels by study and treatment: all p values >0.05, not show

TABLE 12a
Intratumoral gene expression levels by treatment and response in
CONFIRM-1

| | FOLFOX4 + PTK/ZK | | | FOLFOX4 + Placebo | | |
|---|---|---|---|---|---|---|
| | | Re- | Non- | | | Non- |
| Gene | N | sponders | responders | N | Responders | responders |
| LDHA | | | | | | |
| ≦0.36 | 6 | 0 (0%) | 6 (100%) | 2 | 1 (50%) | 1 (50%) |
| >0.36 | 35 | 22 (63%) | 13 (37%) | 39 | 27 (69%) | 12 (31%) |
| P value* | | 0.033 | | | 0.54 | |
| Glut-1^{a} | | | | | | |
| ≦1.50 | 11 | 2 (18%) | 9 (82%) | 13 | 9 (69%) | 4 (31%) |
| >1.50 | 30 | 20 (67%) | 10 (33%) | 28 | 19 (68%) | 9 (32%) |
| P value* | | 0.045 | | | 1.00 | |
| HIF1α | | | | | | |
| ≦1.84 | 26 | 17 (65%) | 9 (35%) | 27 | 19 (70%) | 8 (30%) |
| >1.84 | 15 | 5 (33%) | 10 (67%) | 14 | 9 (64%) | 5 (36%) |
| P value* | | 0.36 | | | 0.73 | |
| VEGF | | | | | | |
| ≦4.16 | 8 | 2 (25%) | 6 (75%) | 7 | 4 (57%) | 3 (43%) |
| >4.16 | 33 | 20 (61%) | 13 (39%) | 34 | 24 (71%) | 10 (29%) |
| P value* | | 0.46 | | | 0.66 | |
| VEGFR1^{b} | | | | | | |
| ≦3.78 | 10 | 1 (10%) | 9 (90%) | 10 | 6 (60%) | 4 (40%) |
| >3.78 | 30 | 20 (67%) | 10 (33%) | 26 | 17 (65%) | 9 (35%) |
| P value* | | 0.012 | | | 1.00 | |
| VEGFR2 | | | | | | |
| ≦1.29 | 8 | 1 (12%) | 7 (82%) | 12 | 8 (67%) | 4 (33%) |
| >1.29 | 33 | 21 (64%) | 12 (36%) | 29 | 20 (69%) | 9 (31%) |
| P value* | | 0.071 | | | 1.00 | |

LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
*Based on the Fisher's exact test but after 2000 bootstrap-like simulations to adjust for selection of optimal cut point for response to FOLFOX4 + PTK/ZK
^{a}p value for interaction = 0.036;
^{b}p value for interaction = 0.031

TABLE 12b
Intratumoral gene expression levels by treatment and response in
CONFIRM-2

| | FOLFOX4 + PTK/ZK | | | FOLFOX4 + Placebo | | |
|---|---|---|---|---|---|---|
| | | Re- | Non- | | | Non- |
| Gene | N | sponders | responders | N | Responders | responders |
| LDHA | | | | | | |
| ≦0.54 | 5 | 4 (80%) | 1 (20%) | 7 | 0 (0%) | 7 (100%) |
| >0.54 | 46 | 10 (22%) | 36 (78%) | 43 | 8 (19%) | 35 (81%) |
| P value* | | 0.064 | | | 0.58 | |
| Glut-1 | | | | | | |
| ≦1.97 | 18 | 2 (11%) | 16 (89%) | 15 | 1 (7%) | 14 (93%) |
| >1.97 | 32 | 11 (34%) | 21 (66%) | 35 | 7 (20%) | 28 (80%) |
| P value* | | 0.49 | | | 0.41 | |
| HIF1α | | | | | | |
| ≦1.18 | 19 | 10 (53%) | 9 (47%) | 17 | 5 (29%) | 12 (71%) |
| >1.18 | 32 | 4 (12%) | 28 (88%) | 33 | 3 (9%) | 30 (91%) |
| P value* | | 0.021 | | | 0.10 | |
| VEGF | | | | | | |
| ≦3.61 | 13 | 7 (54%) | 6 (46%) | 6 | 1 (17%) | 5 (83%) |
| >3.61 | 38 | 7 (18%) | 31 (82%) | 44 | 7 (16%) | 37 (84%) |
| P value* | | 0.13 | | | 1.00 | |
| VEGFR1 | | | | | | |
| ≦3.47 | 11 | 1 (9%) | 10 (91%) | 2 | 0 (0%) | 2 (100%) |
| >3.47 | 36 | 11 (31%) | 25 (69%) | 38 | 5 (13%) | 33 (87%) |
| P value* | | 0.78 | | | 1.00 | |
| VEGFR2 | | | | | | |
| ≦1.55 | 14 | 2 (14%) | 12 (86%) | 13 | 0 (0%) | 13 (100%) |
| >1.55 | 35 | 12 (34%) | 23 (66%) | 37 | 8 (22%) | 29 (78%) |
| P value* | | 0.82 | | | 0.093 | |

LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
*Based on the Fisher's exact test but after 2000 bootstrap-like simulations to adjust for selection of optimal cut point for response to FOLFOX4 + PTK/ZK

TABLE 13a
Intratumoral gene expression levels and progression-free survival by treatment in
CONFIRM-1

| | FOLFOX4 + PTK/ZK | | | FOLFOX4 + Placebo | | |
|---|---|---|---|---|---|---|
| | | Median | | | Median | |
| | | progression-free | | | progression- | |
| | | survival (95% | Relative Risk | | free survival | Relative Risk |
| Gene* | N | CI) | (95% CI) | N | (95% CI) | (95% CI) |
| LDHA | | | | | | |
| ≦0.36 | 6 | 7.5 (1.9, 9.3+) | 1 (Reference) | 2 | 3.5 (3.5, 18.4) | 1 (Reference) |
| >0.36 | 36 | 10.7 (7.6, 11.3) | 0.29 (0.10, | 39 | 9.2 (7.6, 12.7) | 0.99 (0.23, |
| | | | 0.87) | | | 4.23) |
| P value* | | 0.004 | | | 0.99 | |
| Glut-1 | | | | | | |
| ≦1.50 | 11 | 9.3 (7.4, 11.3) | 1 (Reference) | 13 | 9.4 (7.3, 14.9) | 1 (Reference) |
| >1.50 | 31 | 9.4 (7.6, 11.3) | 0.76 (0.36, | 28 | 9.2 (5.7, 13.1) | 0.82 (0.39, |
| | | | 1.60) | | | 1.69) |
| P value* | | 0.42 | | | 0.54 | |
| HIF1α | | | | | | |
| ≦1.84 | 26 | 9.4 (7.5, 11.3) | 1 (Reference) | 27 | 7.6 (7.3, 14.9) | 1 (Reference) |
| >1.84 | 16 | 9.4 (7.4, 11.3) | 1.03 (0.52, | 14 | 9.3 (5.7, 9.4) | 1.28 (0.61, |
| | | | 2.03) | | | 2.66) |
| P value* | | 0.94 | | | 0.46 | |
| VEGF | | | | | | |
| ≦4.16 | 8 | 7.5 (3.5, 9.4) | 1 (Reference) | 7 | 7.6 (7.6, 18.4) | 1 (Reference) |
| >4.16 | 34 | 11.3 (7.6, 11.3) | 0.61 (0.27, | 34 | 9.2 (7.3, 9.4) | 1.59 (0.60, |
| | | | 1.39) | | | 4.17) |
| P value* | | 0.18 | | | 0.29 | |
| VEGFR1 | | | | | | |
| ≦3.78 | 10 | 7.5 (3.5, 9.3) | 1 (Reference) | 10 | 7.6 (5.7, 13.1) | 1 (Reference) |
| >3.78 | 31 | 10.7 (7.6, 11.3) | 0.45 (0.20, | 26 | 7.6 (5.7, 9.4) | 1.41 (0.62, |
| | | | 1.02) | | | 3.18) |
| P value* | | 0.023 | | | 0.33 | |
| VEGFR2 | | | | | | |
| ≦1.29 | 8 | 9.3 (7.5, 11.3) | 1 (Reference) | 12 | 13.1 (7.6, 18.4) | 1 (Reference) |
| >1.29 | 34 | 9.4 (7.6, 11.3) | 0.72 (0.30, | 29 | 7.6 (5.7, 9.4) | 1.79 (0.77, |
| | | | 1.69) | | | 4.19) |
| P value* | | 0.39 | | | 0.12 | |

LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
*Based on the log-rank test

TABLE 13b
Intratumoral gene expression levels and progression-free survival by treatment in
CONFIRM-2

| | FOLFOX4 + PTK/ZK | | | FOLFOX4 + Placebo | | |
|---|---|---|---|---|---|---|
| | | Median | | | Median | |
| | | progression-free | | | progression- | |
| | | survival (95% | Relative Risk | | free survival | Relative Risk |
| Gene* | N | CI) | (95% CI) | N | (95% CI) | (95% CI) |
| LDHA | | | | | | |
| ≦0.54 | 5 | 7.6 (2.7, 29.7+) | 1 (Reference) | 7 | 7.6 (2.0, 9.4+) | 1 (Reference) |
| >0.54 | 47 | 5.3 (2.5, 7.6) | 1.89 (0.71, | 43 | 5.7 (3.9, 5.7) | 1.01 (0.44, |
| | | | 5.07) | | | 2.31) |
| P value* | | 0.11 | | | 0.97 | |
| Glut-1^{a} | | | | | | |
| ≦1.97 | 18 | 7.6 (3.9, 7.6) | 1 (Reference) | 15 | 3.7 (2.1, 5.3) | 1 (Reference) |
| >1.97 | 33 | 2.5 (2.1, 7.6) | 1.47 (0.79, | 35 | 5.7 (5.7, 7.6) | 0.60 (0.31, |
| | | | 2.74) | | | 1.17) |
| P value* | | 0.15 | | | 0.079 | |
| HIF1α | | | | | | |
| ≦1.18 | 19 | 7.6 (7.6, 7.6) | 1 (Reference) | 17 | 7.6 (5.3, 7.6) | 1 (Reference) |
| >1.18 | 33 | 2.5 (2.1, 5.3) | 2.18 (1.16, | 33 | 3.9 (2.1, 5.7) | 1.40 (0.73, |
| | | | 4.11) | | | 2.69) |
| P value* | | 0.002 | | | 0.22 | |
| VEGF | | | | | | |
| ≦3.61 | 13 | 7.6 (2.1, 9.3) | 1 (Reference) | 6 | 3.9 (3.7, 7.6) | 1 (Reference) |
| >3.61 | 39 | 3.9 (2.5, 5.7) | 1.38 (0.72, | 44 | 5.7 (3.9, 5.7) | 1.32 (0.52, |
| | | | 2.66) | | | 3.33) |
| P value* | | 0.26 | | | 0.45 | |
| VEGFR1 | | | | | | |
| ≦3.47 | 11 | 3.9 (2.1, 7.6) | 1 (Reference) | 2 | 2.1 (2.1, 11.3) | 1 (Reference) |
| >3.47 | 37 | 5.6 (2.5, 7.6) | 0.90 (0.44, | 38 | 5.3 (2.1, 5.7) | 2.00 (0.38, |
| | | | 1.85) | | | 10.68) |
| P value* | | 0.75 | | | 0.18 | |
| VEGFR2 | | | | | | |
| ≦1.55 | 14 | 2.7 (1.8, 7.6+) | 1 (Reference) | 13 | 5.7 (2.0, 7.6) | 1 (Reference) |
| >1.55 | 36 | 5.7 (2.5, 7.6) | 0.63 (0.32, | 37 | 5.7 (3.9, 7.6) | 0.93 (0.46, |
| | | | 1.25) | | | 1.89) |
| P value* | | 0.12 | | | 0.81 | |

LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
*Based on the log-rank test
^{a}p value for interaction = 0.038

TABLE 14a
Intratumoral gene expression levels and overall survival by treatment in CONFIRM-1

| | |
|---|---|
| FOLFOX4 + PTK/ZK | FOLFOX4 + Placebo |

| | | Median overall | Relative Risk | | Median overall | Relative Risk |
|---|---|---|---|---|---|---|
| Gene* | N | survival (95% CI) | (95% CI) | N | survival (95% CI) | (95% CI) |
| LDHA | | | | | | |
| ≦0.36 | 6 | 23.3 (23.3, 30.8) | 1 (Reference) | 2 | 3.5 (3.5, 38.0+) | 1 (Reference) |
| >0.36 | 36 | 26.6 (21.4, 33.2) | 0.70 (0.28, 1.73) | 39 | 25.5 (19.4, 46.6+) | 0.95 (0.13, 7.10) |
| P value* | | 0.42 | | | 0.96 | |
| Glut-1 | | | | | | |
| ≦1.50 | 11 | 26.6 (22.7, 33.2) | 1 (Reference) | 13 | 24.3 (17.2, 46.6+) | 1 (Reference) |
| >1.50 | 31 | 27.9 (21.4, 42.7+) | 0.61 (0.29, 1.30) | 28 | 33.9 (20.1, 45.0+) | 0.92 (0.39, 2.16) |
| P value* | | 0.18 | | | 0.84 | |
| HIF1α | | | | | | |
| ≦1.84 | 26 | 26.1 (22.1, 27.9) | 1 (Reference) | 27 | 45.0+ (24.8, 45.0+) | 1 (Reference) |
| >1.84 | 16 | 30.8 (21.1, 35.8) | 0.93 (0.45, 1.94) | 14 | 17.2 (14.3, 37.1) | 2.39 (1.06, 5.38) |
| P value* | | 0.84 | | | 0.026 | |
| VEGF | | | | | | |
| ≦4.16 | 8 | 23.3 (21.4, 31.2) | 1 (Reference) | 7 | 15.9 (11.6, 38.8+) | 1 (Reference) |
| >4.16 | 34 | 26.6 (22.1, 42.7+) | 0.59 (0.26, 1.34) | 34 | 25.5 (20.1, 46.6+) | 0.85 (0.29, 2.49) |
| P value* | | 0.19 | | | 0.76 | |
| VEGFR1 | | | | | | |
| ≦3.78 | 10 | 27.8 (23.3, 33.2) | 1 (Reference) | 10 | 25.5 (15.4, 38.8+) | 1 (Reference) |
| >3.78 | 31 | 26.6 (22.1, 42.7+) | 0.64 (0.30, 1.38) | 26 | 24.3 (17.2, 37.1) | 1.13 (0.45, 2.86) |
| P value* | | 0.24 | | | 0.79 | |
| VEGFR2^{a} | | | | | | |
| ≦1.29 | 8 | 30.8 (23.3, 35.8) | 1 (Reference) | 12 | 45.0+ | 1 (Reference) |
| >1.29 | 34 | 26.2 (21.4, 31.2) | 0.99 (0.42, 2.31) | 29 | 24.3 (15.9, 33.9) | 6.67 (1.59, 28.01) |
| P value* | | 0.98 | | | 0.003 | |

LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
*Based on the log-rank test
^{a}p value for interaction = 0.007

TABLE 14b
Intratumoral gene expression levels and overall survival by treatment in CONFIRM-2

| | FOLFOX4 + PTK/ZK | | | FOLFOX4 + Placebo | | |
|---|---|---|---|---|---|---|
| | | Median OS (95% | Relative Risk | | Median OS (95% | Relative Risk |
| Gene* | N | CI) | (95% CI) | N | CI) | (95% CI) |
| LDHA | | | | | | |
| ≦0.54 | 5 | 9.4 (2.7, 41.1+) | 1 (Reference) | 7 | 14.3 (9.5, 24.2) | 1 (Reference) |
| >0.54 | 47 | 10.7 (7.6, 14.6) | 1.69 (0.52, | 43 | 13.4 (11.3, 18.3) | 0.84 (0.37, |
| | | | 5.48) | | | 1.91) |
| P value* | | 0.37 | | | 0.66 | |
| Glut-1 | | | | | | |
| ≦1.97 | 18 | 14.6 (7.6, 20.1) | 1 (Reference) | 15 | 13.4 (11.1, 18.3) | 1 (Reference) |
| >1.97 | 33 | 10.1 (5.0, 13.4) | 0.92 (0.50, | 35 | 14.3 (10.0, 24.2) | 0.72 (0.37, |
| | | | 1.70) | | | 1.38) |
| P value* | | 0.78 | | | 0.28 | |
| HIF1α | | | | | | |
| ≦1.18 | 19 | 14.6 (9.4, 23.2) | 1 (Reference) | 17 | 18.3 (11.4, 30.7) | 1 (Reference) |
| >1.18 | 33 | 9.4 (5.0, 11.9) | 1.83 (0.96, | 33 | 12.0 (10.0, 16.7) | 1.67 (0.87, |
| | | | 3.48) | | | 3.19) |
| P value* | | 0.053 | | | 0.11 | |
| VEGF | | | | | | |
| ≦3.61 | 13 | 13.4 (7.6, 14.6) | 1 (Reference) | 6 | 9.5 (9.5, 27.5) | 1 (Reference) |
| >3.61 | 39 | 9.4 (7.4, 16.3) | 1.31 (0.65, | 44 | 14.3 (11.7, 21.1) | 0.95 (0.37, |
| | | | 2.67) | | | 2.42) |
| P value* | | 0.44 | | | 0.90 | |
| VEGFR1 | | | | | | |
| ≦3.47 | 11 | 11.9 (10.7, 21.2) | 1 (Reference) | 2 | 6.0 (6.0, 14.7) | 1 (Reference) |
| >3.47 | 37 | 9.1 (5.3, 14.0) | 1.30 (0.62, | 38 | 12.0 (11.1, 18.3) | 0.47 (0.11, |
| | | | 2.76) | | | 2.03) |
| P value* | | 0.48 | | | 0.28 | |
| VEGFR2 | | | | | | |
| ≧1.55 | 14 | 11.9 (5.0, 21.2) | 1 (Reference) | 13 | 24.2 (10.0, 30.4) | 1 (Reference) |
| >1.55 | 36 | 9.4 (7.4, 13.4) | 1.31 (0.66, | 37 | 13.4 (11.1, 18.3) | 1.65 (0.81, |
| | | | 2.63) | | | 3.37) |
| P value* | | 0.43 | | | 0.14 | |

LDHA = lactate dehydrogenase A;
Glut-1 = glucose transporter-1;
HIF1α = hypoxia-inducible factor type-1 alpha;
VEGF (R1) (R2) = vascular endothelial growth factor (type-1 receptor) (type-2 receptor)
*Based on the log-rank test

High expression levels of VEGFR1 were independently associated with response and progression free survival in patients enrolled in CONFIRM-1 and treated with PTK/ZK, indicating a potential role of predicting efficacy of VEGFR TKI therapy. Recently, more effective VEGFR TKI agents have been developed but they have not been shown to increase the efficacy of FOLFOX. This may suggest that a subgroup of patients, identified by molecular predictive markers, such as those discovered herein, is likely to benefit from VEGFR TKI. It is only by the identification of patients who will show optimum benefit that one will be able to increase efficacy of these targeted agents.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying a gastrointestinal cancer patient that is more likely to show responsiveness or less likely to show responsiveness to first line FOLFOX/BV or first line XELOX/BV chemotherapy regimen or equivalent of each thereof, comprising screening a suitable patient cell or tissue sample for at least one genotype of the group of ICAM-1 codon K496E, GRP78 (rs12009), or NFkB CA repeat, wherein
a. (C/C or C/T) for ICAM-1 codon K496E;
b. (C/C or C/T) for GRP78 (rs12009); or
c. (at least 1 allele with ≧24 CA repeats) for NFkB CA repeat, identifies the patient as more likely to show responsive to said therapy or wherein
d. (T/T) for ICAM-1 codon K496E;
e. (T/T) for GRP78 (rs12009); or
f. (two alleles with ≦24 CA repeats) for NFkB CA repeat,
identifies the patient as less likely to show responsive to said therapy.

2. (canceled)

3. A method for selecting a therapy comprising first line FOLFOX/BV or first line XELOX/BV chemotherapy regimen or equivalent of each thereof for a gastrointestinal cancer patient in need thereof, comprising screening a suitable patient cell or tissue sample for at least one genotype of the group:
a. (C/C or C/T) for ICAM-1 codon K496E;
b. (C/C or C/T) for GRP78 (rs12009); or
c. (at least 1 allele with ≧24 CA repeats) for NFkB CA repeat,
wherein the presence of at least one of said genotype selects the patient for said chemotherapy regimen.

4. A method for treating a gastrointestinal cancer patient selected for therapy comprising administration of a first line FOLFOX/BV or first line XELOX/BV chemotherapy regimen or equivalent of each thereof, comprising:
a. screening a suitable patient cell or tissue sample for the presence of at least one genotype of the group:
i. (C/C or C/T) for ICAM-1 codon K496E;
ii. (C/C or C/T) for GRP78 (rs12009); or
iii. (at least 1 allele with ≧24 CA repeats) for NFkB CA repeat,
b. administering an effective amount of said chemotherapy to a patient having at least one genotype identified in step a, thereby treating said patient.

5. The method of claim 1, wherein likelihood of responsiveness is measured by at least one of the group complete response (CR), partial response (PR), stable disease (SD), progressive disease (PD) or progression free survival (PFS).

6. The method of claim 1, wherein the gastrointestinal cancer is a metastatic or non-metastatic cancer selected from the group of rectal cancer colorectal cancer, colon cancer, gastric cancer or esophageal cancer.

7. The method of claim 1, wherein the patient sample comprises tissue or cells selected from non-metastatic tumor tissue, a non-metastatic tumor cell, metastatic tumor tissue, a metastatic tumor cell or peripheral blood lymphocytes.

8. The method of claim 1, wherein the patient sample comprises a non-metastatic tumor cell or tissue.

9. The method claim 1, wherein the patient sample comprises peripheral blood lymphocytes.

10. The method claim 1, wherein the genotype is determined by a method comprising hybridization or PCR.

11. The method claim 1, wherein the genotype is determined by a method comprising PCR-RFLP.

12. (canceled)

13. (canceled)

14. (canceled)

15. (canceled)

16. A method for identifying a gastrointestinal cancer patient that is more likely to experience tumor recurrence or less likely to experience tumor recurrence following surgical resection of a tumor, comprising screening a suitable patient tissue or cell sample for one genotype of the group PAR-1 I-506D, ES G+4349A or IL-8 T-251A polymorphisms, wherein
a. (ins/ins) for PAR-1 I-506D;
b. (A/A) for IL-8 T-251A; or
c. (NA) for ES G+4349A,
identifies the patient as more likely to experience tumor recurrence following surgical resection of a tumor or wherein
d. (del/del or ins/del) for PAR-1 I-506D;
e. (T/T or T/A) for IL-8 T-251A; or
f. (G/G or G/A) for ES G+4349A,
identifies the patient as less likely to experience tumor recurrence following surgical resection of a tumor.

17. (canceled)

18. The method of claim 16, wherein the gastrointestinal cancer is a metastatic or non-metastatic cancer selected from rectal cancer, colorectal cancer, colon cancer, gastric cancer or esophageal cancer.

19. The method of claim 16, wherein the patient sample comprises tissue or cells selected from non-metastatic tumor tissue, a non-metastatic tumor cell, metastatic tumor tissue, a metastatic tumor cell, peripheral blood lymphocytes or whole blood.

20. The method of claim 16, wherein the patient sample comprises peripheral blood lymphocytes.

21. The method claim 16, wherein the genotype is determined by a method comprising hybrization or PCR.

22. The method claim 16, wherein the genotype is determined by a method comprising PCR-RFLP.

23. A method for identifying a stage II colon cancer patient that is less likely to experience tumor recurrence or less likely to experience tumor recurrence following 5-FU based adjuvant chemotherapy regimen or equivalent thereof, comprising screening a suitable patient cell or tissue sample for at least one genotype of the group IL-1β C+3954T, IL-1Ra VNTR or VEGF G-634C, wherein
a. (C/C or C/T) for IL-1β C+3954T;
b. (4 repeats/4 repeats or 2 repeats/2 repeats) for IL-1Ra VNTR; or
c. (G/G) for VEGF G-634C,
identifies the patient as less likely to experience tumor recurrence following said therapy or wherein
d. (T/T) for IL-1β C+3954T;
e. (at least one allele with >4 repeats) for IL-1Ra VNTR; or
f. (C/C or C/G) for VEGF G-634C,
identifies the patient as more likely to experience tumor recurrence following said therapy.

24. (canceled)

25. A method for selecting a therapy comprising 5-FU based adjuvant chemotherapy regimen or equivalent thereof for a stage II colon cancer patient in need thereof, comprising screening a suitable patient cell or tissue sample for the presence of a genotype (C/C or C/T) in IL-1β C+3954T; (4 repeats/4 repeats or 2 repeats/2 repeats) in IL-1Ra VNTR; or (G/G) in VEGF G-634C, wherein the presence of said genotype selects said patient for said chemotherapy.

26. A method for treating a stage II colon cancer patient selected for therapy comprising administration of a 5-FU based adjuvant chemotherapy regimen or equivalent thereof, comprising:
a. screening a suitable patient cell or tissue sample for the presence of a genotype (C/C or C/T) for IL-1β C+3954T; (4 repeats/4 repeats or 2 repeats/2 repeats) for IL-1Ra VNTR; or (G/G) for VEGF G-634C, and
b. administering an effective amount of said chemotherapy to a patient having a genotype identified in step a, thereby treating said patient.

27. The method of claim 23, wherein tumor recurrence is measured by risk of tumor recurrence, time to tumor recurrence or disease free survival after treatment with said therapy.

28. The method of claim 23, wherein the patient sample comprises tissue or cells selected from non-metastatic tumor tissue, a non-metastatic tumor cell, metastatic tumor tissue, a metastatic tumor cell, peripheral blood lymphocytes or whole blood.

29. The method of claim 23, wherein the patient sample comprises a non-metastatic tumor cell or tissue.

30. The method of claim 23, wherein the patient sample comprises peripheral blood lymphocytes.

31. The method of claim 23, wherein the genotype is determined by a method comprising hybridization or PCR.

32. The method of claim 23, wherein the genotype is determined by a method comprising PCR-RFLP.

33. The method of claim 23, wherein the 5-FU based adjuvant chemotherapy comprises FOLFOX (5-FU, leucovorin and oxaliplatin); FOLFIRI (5-FU, leucovorin and irinotecan) or 5-FU and leucovorin.

34.-71. (canceled)
